(19) United States
(12) Patent Application Publication
(10) Pub. No.: US 2024/0293686 A1
(43) Pub. Date: Sep. 5, 2024

(54) ELECTRO-IONIC MASK CONTROL DEVICES, SYSTEMS, AND METHODS FOR IMPROVED PROTECTION FROM AIRBORNE BIO-PATHOGENS

(71) Applicant: Julian Henley, New Orleans, LA (US)

(72) Inventor: Julian Henley, New Orleans, LA (US)

(21) Appl. No.: 18/593,850

(22) Filed: Mar. 1, 2024

Related U.S. Application Data
(60) Provisional application No. 63/552,034, filed on Feb. 9, 2024.
(60) Provisional application No. 63/487,787, filed on Mar. 1, 2023.

(51) Int. Cl. A62B 23/02; A61L 9/22; A62B 7/10; A62B 18/08

(52) U.S. Cl. CPC A62B 23/025; A61L 9/22; A62B 7/10; A62B 18/084; A61L 2209/14

(57) ABSTRACT

Aspects of the present disclosure include a hybrid filter for use with an activated carbon-based filter and attachable to a mask. The hybrid filter may include a chamber configured to receive the activated carbon-based, an electro-ionic filter, and a Faraday cage. The electro-ionic filter is in series with the material-based filtration element and includes a plurality of electrodes arranged in series with each other. The electrodes of the plurality of electrodes are evenly spaced apart from each other in an alternating positive-polarity negative-polarity sequence. The Faraday cage surrounds at least the electro-ionic filter.

### CROSS REFERENCE TO RELATED APPLICATION

This application claims priority from U.S. Provisional Patent Appln. No. 63/552,034 filed on Feb. 9, 2024 and U.S. Provisional Patent Appln. No. 63/487,787 filed on Mar. 1, 2023, the entirety of each is incorporated by reference herein. This application also incorporates by reference herein the entireties of the disclosures of the following: Int'l Patent Appln. No. PCT/US2021/022386 filed on Mar. 15, 2021, Int'l Appln. No. PCT/US2022/071175 filed on Mar. 15, 2022, U.S. Provisional Patent Appln. No. 62/988,991 filed on Mar. 13, 2020, U.S. Provisional Patent Appln. No. 63/027,746 filed on May 20, 2020, U.S. Provisional Patent Appln. No. 63/043,424 filed on Jun. 24, 2020, U.S. Provisional Patent Appln. No. 63/044,768 filed on Jun. 26, 2020, U.S. Provisional Patent Appln. No. 63/063,968 filed on Aug. 11, 2020, U.S. Provisional Patent Appln. No. 63/113,598 filed on Nov. 13, 2020, U.S. Provisional Patent Appln. No. 63/230,273 filed on Aug. 6, 2021, and U.S. Provisional Patent Appln. No. 63/310,810 filed on Feb. 16, 2022, and U.S. Pat. No. 6,901,930 filed on Oct. 28, 2002.

### FIELD OF THE INVENTION

This application relates to devices and methods for improved protection from airborne bio-pathogens and toxins. In particular, this application relates to wearable devices and methods of using wearable devices for particle capture and deactivation.

### BACKGROUND OF THE INVENTION

For many years airway protection has been based on filtration technology employing particle entrapment within interposed dense fiber layers, which can be considered a material-based filtration element. For biohazardous chemical protection, material-based filtration elements can have additional layers of activated charcoal because of the molecular bonding and absorptive capability of activated charcoal.

Activated carbon has known limits to its absorptive capacity, thus requiring that such filters be periodically replaced before their absorptive capacity is exceeded. Unfortunately, due to the many variables involved, including the types and concentrations of the biohazardous airborne agents in the air, the concentrations of dust in the air, the temperature and humidity of the ambient environment, and the varying respiratory rates of a person employing the activated carbon filter, it is impossible to predict how long the activated carbon filter will adequately protect the person in a given situation. Any miscalculation with respect to whether or not an activated carbon filter is approaching, or has exceeded its absorptive capacity, can have catastrophic consequences for the person employing the activated charcoal filter.

A secondary problem with respect to activated charcoal filters having been employed in an environment containing biohazardous airborne agents is properly disposing of such filters, including achieving the adequate chemical degradation of the toxic agents trapped in the filter. Handlers of such contaminated filters may be inadvertently exposed to the toxic agents that remain embedded in the previously used filter modules and associated equipment.

Consequently, there is a need in the art for devices, systems and methods that address these serious issues facing the modern warfighter, first responder and those working in healthcare, labs and other potentially hazardous environments.

### SUMMARY OF THE INVENTION

Reference will now be made in detail to the exemplary embodiments, which are illustrated in the accompanying drawings. Wherever possible, the same reference numbers will be used throughout the drawings to refer to the same or like parts.

In a first exemplary embodiment of the present invention, a hybrid filter is disclosed. The hybrid filter can be used with an activated carbon-based filter and attachable to a mask. The hybrid filter includes a chamber, an electro-ionic filter, and a Faraday cage. The chamber can be configured to receive the activated carbon-based filter. The electro-ionic filter can be in series with the chamber and include multiple electrodes arranged in series with each other. The electrodes of the multiple electrodes can be evenly spaced apart from each other in an alternating positive-polarity negative-polarity sequence. The Faraday cage can surround at least the electro-ionic filter.

In one version of the hybrid filter, a longitudinal axis of the chamber is coaxial with a longitudinal axis of the electro-ionic filter.

In one version of the hybrid filter, the chamber and the electro-ionic filter are disposed in a housing. The housing can form at least a portion of the Faraday cage.

In one version of the hybrid filter, the housing includes at least one of a metal or a polymer coated with a conductive coating.

In one version of the hybrid filter, the chamber and the electro-ionic filter are disposed in a housing. The housing can include a neck configured to threadably couple to a port of the mask.

In one version of the hybrid filter, the chamber and the electro-ionic filter are disposed in a housing. An intake grille can be disposed at a first end of the housing and an exit grille can be disposed at a second end of the housing. The intake grille and the exit grille can form at least a portion of the Faraday cage.

In one version of the hybrid filter, a longitudinal axis of the intake grille is colinear with a longitudinal axis of the exit grille.

In one version of the hybrid filter, the chamber is configured to also receive a particulate filter such that the particulate filter would be in series with the activated carbon-based filter.

In one version of the hybrid filter, the hybrid filter includes an ozone scrubber in series with the chamber.

In one version of the hybrid filter, the hybrid filter includes the activated carbon-based filter, which can be configured to retain organic compounds. The electro-ionic filter can be configured to generate ozone at a level that degrades the organic compounds via oxidative degradation.

In one version of the hybrid filter, the hybrid filter includes the activated carbon-based filter, which can have an absorptive capacity. The electro-ionic filter can be configured to regenerate the absorptive capacity by controlled oxidation at ambient temperature.

In one version of the hybrid filter, a concurrent operating voltage of the multiple electrodes is between approximately 2 kV and approximately 18 kV, between approximately 5 kV and approximately 15 kV, and/or between approximately 8 kV and approximately 12 kV.

In one version of the hybrid filter, an applied voltage range between a pair of the multiple electrodes is between approximately 1 kV and approximately 8 kV.

In one version of the hybrid filter, the multiple electrodes are arranged in a P-N-P, P-N-P-N-P, or P-N-P-N-P-N-P arrangement.

In one version of the hybrid filter, the multiple electrodes include a first electrode module and a second electrode module. The first electrode module can be arranged in a P-N-P arrangement and the second electrode module can be arranged in a P-N-P arrangement.

In one version of the hybrid filter, the multiple electrodes includes at least one positive electrode arranged as a grid defining an open area for airflow therethrough and at least one negative electrode defining an open area for airflow therethrough. The open area defined by the at least one negative electrode can be greater than the open area defined by the at least one positive electrode.

In one version of the hybrid filter, multiple electrodes includes at least one positive electrode and at least one negative electrode. The at least one positive electrode can include a wire arranged in a grid and defining a diameter The at least one negative electrode can include a wire arranged in a grid and defining a diameter. The diameter of the wire of the at least one positive electrode can be greater than the diameter of the wire of the at least one negative electrode.

In one version of the hybrid filter, each of the multiple electrodes includes an open grid extending across a chamber and perpendicular to sidewalls of a housing containing the electro-ionic filter therein.

In one version of the hybrid filter, the multiple electrodes includes at least a starting positive electrode, an ending positive electrode, and at least one negative electrode disposed between the starting positive electrode and the ending positive electrode. The starting positive electrode and the ending positive electrode can form end portions of the Faraday cage encapsulating the at least one negative electrode.

In one version of the hybrid filter, the multiple electrodes includes at least one positive electrode acting as a positive collector and at least one negative electrode acting as a negative emitter.

In one version of the hybrid filter, the multiple electrodes are flat extending across the electro-ionic filter.

In one version of the hybrid filter, the multiple electrodes are curved extending across the electro-ionic filter.

In one version of the hybrid filter, each of the multiple electrodes includes an occluded portion and a non-occluded portion configured such that airflow is circuitously routed through the electro-ionic filter.

In one version of the hybrid filter, a spacer section is disposed between each offset pair of the plurality of electrodes. Each spacer section can have an outer surface abutting an inner surface of a housing. The electro-ionic filter can be disposed in the housing.

In a second exemplary embodiment of the present invention, a hybrid filter is disclosed. The hybrid filter can be configured for use with an activated carbon-based filter and attachable to a mask. The hybrid filter can include a chamber, an electro-ionic filter, and a Faraday cage. The chamber can be configured to receive the activated carbon-based filter. The electro-ionic filter can be in series with the chamber and include multiple electrode modules arranged in parallel with each other. Each electrode module can include a cylindrical positive collector electrode encompassing a negative emitter electrode extending coaxial with a longitudinal axis of the cylindrical positive collector electrode and equally spaced-apart from an inner cylindrical surface of the cylindrical positive collector electrode in all radial directions. The Faraday cage can surround at least the electro-ionic filter.

In one version of the hybrid filter, the longitudinal axis of the cylindrical positive collector electrode is substantially parallel to a longitudinal axis of the chamber.

In one version of the hybrid filter, the chamber is configured to also receive a particulate filter such that the particulate filter would be in series with the activated carbon-based filter.

In one version of the hybrid filter, the hybrid filter includes an ozone scrubber in series with the chamber.

In one version of the hybrid filter, the hybrid filter includes the activated carbon-based filter. The activated carbon-based filter can be configured to retain organic compounds. The electro-ionic filter can be configured to generate ozone at a level that degrades the organic compounds via oxidative degradation.

In one version of the hybrid filter, the hybrid filter includes the activated carbon-based filter. The activated carbon-based filter can have an absorptive capacity. The electro-ionic filter can be configured to regenerate the absorptive capacity by controlled oxidation at ambient temperature.

In a third exemplary embodiment of the present invention, a filter is disclosed. The filter can be configured for use with an activated carbon-based filter and attachable to a mask. The filter can include an ozonated chamber, an ozone generator, and an ozone scrubber. The ozonated chamber can be defined between the ozone generator and the ozone scrubber. The ozone generator can be configured to deliver a concentration of ozone into the ozonated chamber. The ozone scrubber can be configured to reduce the concentration of ozone leaving the ozonated chamber. The ozonated chamber can be configured to receive the activated carbon-based filter.

In one version of the filter, the filter includes the activated carbon-based filter. The activated carbon-based filter can be configured to retain organic compounds passing through the ozonated chamber. The ozone generator can be configured to deliver a concentration of ozone that degrades the organic compounds by oxidative degradation.

In one version of the filter, the filter includes the activated carbon-based filter. The activated carbon-based filter can have an absorptive capacity. The ozone generator can be configured to deliver a concentration of ozone that regenerates the absorptive capacity by controlled oxidation at ambient temperature.

In one version of the filter, the ozone generator includes one or more electrodes configured to generate ozone.

In one version of the filter, the ozone generator is coupled to a housing that defines at least a portion of the ozonated chamber.

In one version of the filter, the filter includes a humidity source configured to deliver humidity into the ozonated chamber.

In one version of the filter, the filter includes a humidity source disposed within the ozonated chamber.

In one version of the filter, the filter includes the activated carbon-based filter. The activated carbon-based filter can include water and/or a buffering agent.

In a fourth exemplary embodiment of the present invention, a filter is disclosed. The filter can be attachable to a mask. The filter can include an ozone generator, an activated carbon-based filter, and an ozone scrubber. The ozone generator can be configured to deliver ozone into an airflow at a first level of ozone. The activated carbon-based filter can be in series with the ozone generator and configured to capture contaminates from the airflow. The activated carbon-based filter can be exposed to the ozone within the airflow. The ozone scrubber can be in series with the activated carbon-based filter and configured to reduce the ozone in the airflow to a second level of ozone. The second level of ozone can be less than the first level of ozone.

In one version of the filter, the first level of ozone causes oxidation of at least a portion of the contaminates that are captured.

In one version of the filter, the first level of ozone is greater than 0.2 ppm.

In one version of the filter, the ozone scrubber decomposes at least a portion of the ozone within the airflow.

In one version of the filter, the second level of ozone is less than 0.1 ppm.

In a fifth exemplary embodiment of the present invention, a filter is disclosed. The filter can be attachable to a mask. The filter can include a housing, an ozone generator, an activated carbon-based filter, and an ozone scrubber. The housing can define a chamber configured to receive airflow therethrough. The ozone generator can be coupled to the housing and configured to deliver ozone into the airflow. The activated carbon-based filter can be disposed within the chamber and configured to capture at least a portion of contaminates within the airflow. The activated carbon-based filter can be downstream from the ozone generator. The ozone scrubber can be configured to decompose at least a portion of the ozone in the airflow. The ozone scrubber can be downstream from the activated carbon-based filter.

In one version of the filter, the ozone generator generates a level of ozone to neutralize organophosphates.

In one version of the filter, the ozone generator generates a level of ozone that oxidizes and degrades toxins retained by the activated carbon-based filter.

In one version of the filter, the filter includes a particulate filter in series with the activated carbon-based filter.

In one version of the filter, the ozone scrubber includes at least one of cobalt oxide or manganese oxide.

In one version of the filter, the ozone generator includes an anode and a cathode.

In one version of the filter, the ozone generator includes a positive electrode and a negative electrode.

In one version of the filter, the activated carbon-based filter is impregnated with water.

In one version of the filter, the filter includes a reservoir coupled to the housing. The reservoir can be configured to retain water such that the water humidifies the activated carbon-based filter.

In one version of the filter, the activated carbon-based filter can include a pH buffering agent.

In a sixth exemplary embodiment of the present invention, a hybrid filter is disclosed. The hybrid filter can include an activated carbon-based filter, an emitter, and a collector. The emitter can be disposed within the activated carbon-based filter. The collector can encompass the emitter. The emitter and collector can be configured to provide a current through the activated carbon-based filter.

In one version of the filter, the collector is a housing of the hybrid filter.

In one version of the filter, the collector radially surrounds the emitter.

In one version of the filter, the emitter is longitudinally disposed along a central axis of the hybrid filter.

In one version of the filter, the filter includes an electro-ionic filter. The activated carbon-based filter can be configured to retain organic compounds. The electro-ionic filter can be configured to generate ozone at a level that degrades the organic compounds via oxidative degradation.

In one version of the filter, the filter includes an electro-ionic filter in series with the activated carbon-based filter.

In a seventh exemplary embodiment of the present invention, a hybrid filter is disclosed. The hybrid filter can be configured to be attachable to a mask and for use with a filter insert including an inlet to receive airflow and an outlet to discharge the airflow. The filter insert can include an activated carbon-based filter disposed therein. The hybrid filter can include a housing, a housing cap, and a chamber. The a housing cap can be removably couplable to the housing. The chamber can be configured to receive the filter insert. The filter insert can be removably couplable to the housing and/or the housing cap. The hybrid filter can include an electro-ionic filter and/or ozone generator in series with the chamber.

In one version of the hybrid filter, the housing can include an air inlet and the housing cap can include an air outlet.

In one version of the hybrid filter, the housing and the housing cap can each contain corresponding threads. The housing cap can be removably coupled to the housing when the corresponding threads are rotatably mated together.

In one version of the hybrid filter, the filter insert and the housing cap each contain corresponding threads. The filter insert can be removably coupled to the housing cap when the corresponding threads are rotatably mated together.

In one version of the hybrid filter, the electro-ionic filter can include an emitter at least partially surrounded by a collector.

In one version of the hybrid filter, the filter insert can be manufactured by Avon protection or an equivalent.

In one version of the hybrid filter, the filter insert can include a CBRNCF50 Filter, a FM61EU CBRN Filter, a GPCF50 CBRN Filter, a CFP100 Particulate Filter, and/or a CTCF50 Riot Agent Filter.

In one version of the hybrid filter, the hybrid filter includes a gasket disposed between the filter insert and the housing and/or the filter insert and the housing cap when the filter insert is removably coupled thereto.

In an eighth exemplary embodiment of the present invention, a hybrid filter is disclosed. The hybrid filter can have a housing configured to removably receive a filter insert therein such that the filter insert is removably coupled to the housing. The filter insert can have an inlet and a neck defining an outlet. The hybrid filter can include a first housing portion and a section housing portion. The first housing portion can include a neck defining an airflow exit. The neck of the first housing portion can be configured to receive the neck of the filter insert therein such that the airflow exit of the first housing portion is coaxial with the outlet of the filter insert. The second housing portion can define an airflow inlet. The second housing portion can be configured to removably couple to the first housing portion. The hybrid filter can include an electro-ionic filter and/or an ozone generator in series with the filter insert when the filter insert is removably coupled with the housing.

In one version of the hybrid filter, the hybrid filter includes the filter insert.

In one version of the hybrid filter, the filter insert can include an activated carbon-based filter disposed therein.

In one version of the hybrid filter, the filter insert can be manufactured by Avon protection or an equivalent.

In one version of the hybrid filter, the filter insert can include a CBRNCF50 Filter, a FM61EU CBRN Filter, a GPCF50 CBRN Filter, a CFP100 Particulate Filter, and/or a CTCF50 Riot Agent Filter.

In one version of the hybrid filter, the neck of the first housing portion can include internal threads configured to rotatably mate with external threads on the neck of the filter insert.

In one version of the hybrid filter, the neck of the first housing portion and the neck of the filter insert can be configured for a male/female or nested arrangement when the neck of the first housing portion receives the neck of the filter insert.

In one version of the hybrid filter, the airflow exit of the first housing portion can be coaxial with the airflow inlet of the second housing portion when the second housing portion is removably coupled to the first housing portion.

In one version of the hybrid filter, the first housing portion and the second housing portion can include corresponding threads that rotatably mate together when the second housing portion is removably coupled to the first housing portion.

In one version of the hybrid filter, the second housing portion can include the electro-ionic filter and/or ozone generator disposed therein.

In one version of the hybrid filter, the hybrid filter includes a gasket disposed between the filter insert and the first housing portion when the filter insert is removably coupled to the first housing portion.

In a ninth exemplary embodiment of the present invention, a housing cap is disclosed. The housing cap can be configured to receive a filter insert therein and configured to couple to a gas mask. The housing cap can include a neck and a body. The neck can have internal threads and external threads. The internal threads can be configured to rotatably mate with corresponding external threads on a neck of the filter insert such that the filter insert is removably coupled to the neck of the housing cap. The body can extend from the neck. The body can include an attachment mechanism configured to removably couple the body of the housing cap to a housing.

In one version of the housing cap, the attachment mechanism is a threaded connection.

In one version of the housing cap, the housing cap can include a gasket disposed on an internal surface of housing cap such that the gasket is positioned between the filter insert and the housing cap when the filter insert is removably coupled to the housing cap.

In one version of the housing cap, at least a portion of sidewalls of the body abut at least a portion of the filter insert when the filter insert is removably coupled to the housing cap.

In one version of the housing cap, at least a portion of a base of the body abuts at least a portion of the filter insert when the filter insert is removably coupled to the housing cap.

In a tenth exemplary embodiment of the present invention, a method is disclosed. The method can be a method of factory assembling a hybrid filter. The method can include removably coupling a filter insert to a first housing portion such that an outlet of the filter insert is coaxial with an airflow exit of the first housing portion. The method can include removably coupling a second housing portion to the first housing portion. The second housing portion can include an electro-ionic filter and/or an ozone generator disposed therein.

In one version of the method, the electro-ionic filter and/or the ozone generator can be arranged in series with the filter insert when the filter insert and the second housing portion are both removably coupled to the first housing portion.

In one version of the method, removably coupling the filter insert to the first housing portion can include rotatably mating external threads on a neck of the filter insert with corresponding internal threads in a neck the first housing portion.

In one version of the method, removably coupling the second housing portion to the first housing portion can include rotatably mating corresponding threads on each of the second housing portion and the first housing portion.

In one version of the method, the method can include positioning a gasket between the filter insert and the first housing portion.

In one version of the method, the filter insert can be manufactured by Avon protection or an equivalent.

In one version of the method, the filter insert can include a CBRNCF50 Filter, a FM61EU CBRN Filter, a GPCF50 CBRN Filter, a CFP100 Particulate Filter, and/or a CTCF50 Riot Agent Filter.

In one version of the method, the airflow exit of the first housing portion can be coaxial with an airflow inlet of the second housing portion when the second housing portion is removably coupled to the first housing portion.

In one version of the method, removably coupling the filter insert to the first housing portion can include abutting at least a portion of the filter insert to sidewalls of the first housing portion.

In one version of the method, removably coupling the filter insert to the first housing portion can include abutting at least a portion of the filter insert to a base of the first housing portion.

In an eleventh exemplary embodiment of the present invention, a method is disclosed. The method can be a method of field assembling a hybrid filter. The method can include removably coupling a first housing portion of the hybrid filter to a gas mask. The hybrid filter can include a filter insert removably coupled to the first housing portion such that an outlet of the filter insert is coaxial with an airflow exit of the first housing portion. The hybrid filter can include a second housing portion removably coupled to the first housing portion. The second housing portion can include an electro-ionic filter and/or an ozone generator disposed therein.

In one version of the method, the method can include removably coupling the filter insert to the first housing portion.

In one version of the method, removably coupling the filter insert to the first housing portion can include rotatably mating external threads on a neck of the filter insert with corresponding internal threads in a neck the first housing portion.

In one version of the method, removably coupling the filter insert to the first housing portion can include abutting at least a portion of the filter insert to sidewalls of the first housing portion.

In one version of the method, removably coupling the filter insert to the first housing portion can include abutting at least a portion of the filter insert to a base of the first housing portion.

In one version of the method, the method can include removably coupling the second housing portion to the first housing portion.

In one version of the method, removably coupling the second housing portion to the first housing portion can include rotatably mating corresponding threads on each of the second housing portion and the first housing portion.

In one version of the method, the airflow exit of the first housing portion can be coaxial with an airflow inlet of the second housing portion when the second housing portion is removably coupled to the first housing portion.

In one version of the method, the method can include removing the second housing portion from the first housing portion.

In one version of the method, the method can include removably coupling a new second housing portion to the first housing portion. The new second housing portion can include an electro-ionic filter and/or an ozone generator disposed therein.

In one version of the method, the electro-ionic filter and/or the ozone generator can be arranged in series with the filter insert when the filter insert and the second housing portion are both removably coupled to the first housing portion.

In one version of the method, the method can include positioning a gasket between the filter insert and the first housing portion.

In one version of the method, the filter insert can be manufactured by Avon protection or an equivalent.

In one version of the method, the filter insert can include a CBRNCF50 Filter, a FM61EU CBRN Filter, a GPCF50 CBRN Filter, a CFP100 Particulate Filter, and/or a CTCF50 Riot Agent Filter.

In a twelfth exemplary embodiment of the present invention, an electro-ionic component is disclosed. The electronic component can be attachable to a housing of a filter element. The filter element can include an activated carbon-based filter and/or a particulate filter surrounded by the housing. The housing can include an airflow exit and an airflow intake opposite the airflow exit. The airflow exit can be adapted to removably couple to a gas mask. The electronic component can include an electro-ionic filter and/or an ozone generator supported within a casing. The casing can be adapted to removably attach to the housing such that the electronic component is adjacent the airflow intake and the electro-ionic filter and/or ozone generator are in series with the activated carbon-based filter and/or the particulate filter.

In one version of the electronic component, the electronic component can include mechanical attachment features that facilitate the removable attachment of the casing to the housing. The mechanical attachment features can include threads, bolts, latches, screws, interference fitments, pins, keys, bayonet coupling arrangements, and/or twist-on-off engagements.

### BRIEF DESCRIPTION OF THE DRAWINGS

The foregoing summary, as well as the following detailed description of preferred embodiments of the invention, will be better understood when read in conjunction with the appended drawings. For purposes of illustrating the invention, there are shown in the drawings embodiments which are presently preferred. It should be understood, however, that the invention is not limited to the precise arrangements and instrumentalities shown. In the drawings:

FIG. 1 is a front perspective view of an overall filtration system employing an M40A1 Field Protective Gas Mask and a hybrid filter operably connected thereto.

FIG. 2 is the same view as FIG. 1, except the hybrid filter has been sectioned to reveal some of its internal components.

FIG. 3 is an enlarged view of the sectioned hybrid filter from FIG. 2.

FIG. 4 is the same view of the sectioned hybrid filter as FIG. 3, except shown with the material-based filtration element removed.

FIG. 5 is a top-side perspective view of the hybrid filter of FIG. 2.

FIG. 6 is a top-side perspective view of the hybrid filter of FIG. 2 from a perspective different from that of FIG. 5.

FIG. 7 is a bottom-side perspective view of the hybrid filter of FIG. 2.

FIG. 8 is a side elevation of the hybrid filter of FIG. 2.

FIG. 9 is a cross-sectional perspective view of the hybrid filter as taken along section line 9-9 in FIG. 6.

FIG. 10 is a top plan view of an electrode configuration representative of that employed for the positive electrodes.

FIG. 11 is a top plan view of an electrode configuration representative of that employed for the negative electrodes.

FIG. 12 is a side elevation of a hybrid filter employing an alternative electro-ionic filter having curved electrodes.

FIG. 13 is a cross-sectional perspective view of the hybrid filter as taken along section line 9-9 in FIG. 6, but of another embodiment employing structures modifying the airflow pathway through the stack of electrodes, thereby prolonging airflow contact with the positive collector electrodes.

FIG. 14 is a cross-sectional perspective view of the hybrid filter as taken along section line 9-9 in FIG. 6, but of the embodiment of FIG. 13 and illustrating the airflow through the stack of electrodes.

FIG. 15 is a side elevation of a hybrid filter employing an alternative electro-ionic filter having a plurality of cylindrical electrode modules arranged in parallel relative to each other.

FIG. 16 is a cross-section through the hybrid filter as taken along section line 16-16 in FIG. 15.

FIG. 17 is a top-side perspective view of a filter having an ozone generator.

FIG. 18 is a cross-sectional perspective view of the filter and ozone generator as taken along section lines 18-18 in FIG. 17.

FIG. 19 is the same view as FIG. 18, but includes an example of an ozone generator.

FIG. 20 is the same view as FIG. 18, but includes another example of an ozone generator.

FIG. 21 is an example computing device, in accordance with embodiments of the disclosure.

FIG. 22 is a flowchart illustrating modulation of the device, in accordance with embodiments of the disclosure.

FIG. 23 is a bottom-side perspective view of a hybrid filter.

FIG. 24 is a cross-sectional perspective view of the hybrid filter as taken along section line 24-24 in FIG. 23.

FIG. 25 is an exploded view of the hybrid filter of FIG. 23.

FIG. 26 is a bottom-side perspective view of a hybrid filter.

FIG. 27 is a cross-sectional perspective view of the hybrid filter as taken along section line 27-27 in FIG. 26.

FIG. 28 is an exploded view of the hybrid filter of FIG. 26.

FIG. 29 is an assembled view of an electro-ionic filter.

FIG. 30 is an exploded view of the electro-ionic filter of FIG. 29.

FIG. 31 is a bottom-side perspective view of a hybrid filter.

FIG. 32 is a cross-sectional perspective view of the hybrid filter as taken along section line 32-32 in FIG. 31.

FIG. 33 is an exploded view of the hybrid filter of FIG. 31.

FIG. 34 is a bottom-side perspective view of a hybrid filter.

FIG. 35 is a bottom-side perspective view of a hybrid filter.

FIG. 36 is a cross-sectional perspective view of the hybrid filter as taken along section line 36-36 in FIG. 35.

FIG. 37 is an exploded view of the hybrid filter of FIG. 35.

FIG. 38 illustrates a cross-sectional view of the activated charcoal of the hybrid filter of FIG. 35 with current flowing therethrough.

FIG. 39 illustrates an example computing system that may implement various systems and methods discussed herein.

FIG. 40 is a flowchart illustrating a method of factory assembling a modular hybrid filter, in accordance with embodiments of the disclosure.

FIG. 41 is a flowchart illustrating a method of field assembling a modular hybrid filter, in accordance with embodiments of the disclosure.

### DETAILED DESCRIPTION

Reference will now be made in detail to the exemplary embodiments, which are illustrated in the accompanying drawings. Wherever possible, the same reference numbers will be used throughout the drawings to refer to the same or like parts.

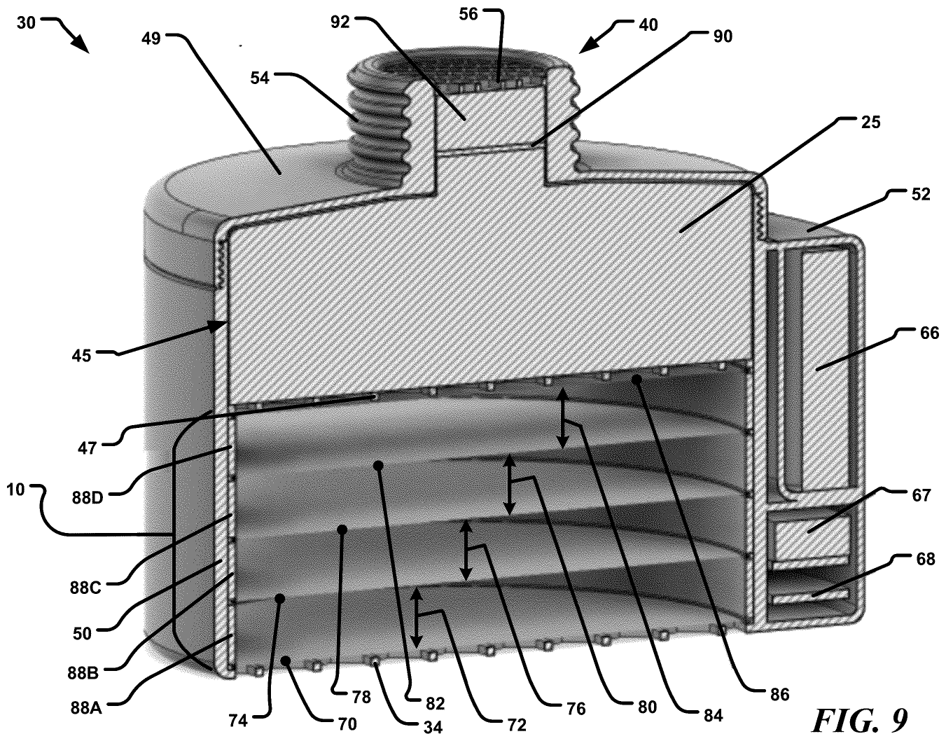

Described herein is an electro-ionic filter 10 (see FIG. 9) that employs ionization and electrostatic precipitation technologies to not only trap airborne biohazards but also degrade and neutralize those airborne biohazards in real time during use. Further, the electro-ionic filter 10, with its enhanced oxidative self-sanitizing capability, can self-sanitize when the acute need for use has ended.

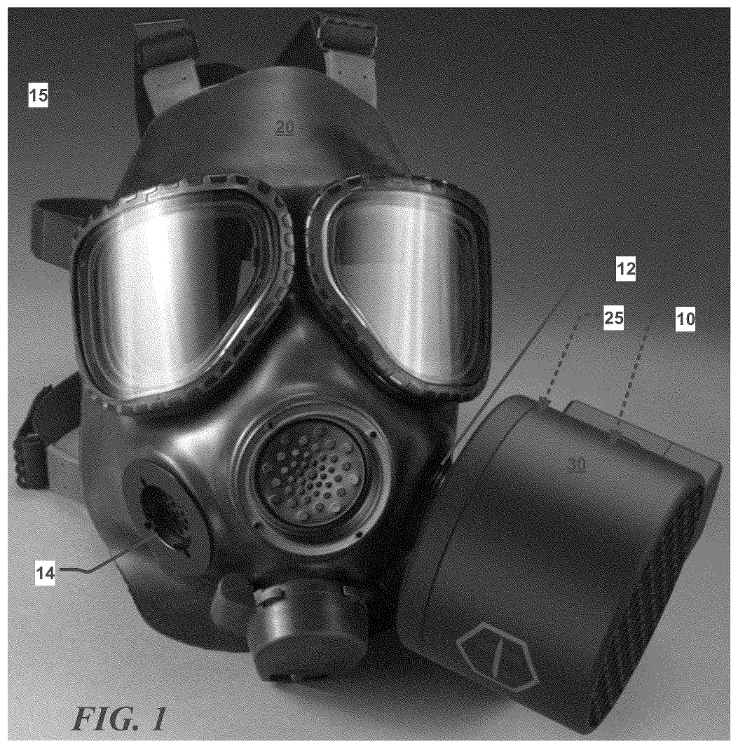

In some embodiments, the electro-ionic filter 10 can be employed as the sole filtration element of an overall filtration system 15 employing a mask 20 such as, for example, the U.S. ARMY's M40A1 Field Protective Gas Mask 20 (see FIG. 1), the U.S. Military's M50 Series Joint Service General Purpose Mask (JSGPM), or any of a host of other masks used by NATO or other militaries around the world employing canister style filtration elements. The electro-ionic filter 10 can also be used with face masks, half masks, and gas masks employed by civilians and civilian authorities.

In other embodiments, the electro-ionic filter 10 can be combined with a filtration element (such as an activated carbon-based filter 25) to form a hybrid filter 30 as shown in FIGS. 1, 2 and 9, for example. Examples of such common existing filtration elements include canister filters, or the material-based filtration components thereof found in 40 mm canister filters employed with the above-mentioned masks. Often the material-based filtration components of such canister filters employ interposed dense fiber layers and/or activated carbon to facilitate filtration of toxic elements from the airstream of the user.

In such a combined hybrid arrangement 30, the electro-ionic filter 10 greatly enhances the life and efficacy of the activated carbon-based filter 25 by virtue of the self-sanitization and chemical degradation capability provided by the electro-ionic filter 10 with respect to toxins that are normally retained by the activated carbon-based filter 25. In practical effect, because of the self-sanitization and chemical degradation capability provided by the electro-ionic filter 10, the absorptive capability of the activated carbon-based filter 25 is reactivated between uses, the previously retained toxins have been neutralized and no longer presenting a toxic risk to potential handlers or re-users of such filters or the rest of the mask 20.

It should be noted here that the activated carbon-based filter 25 can include an activated carbon (e.g., an activated carbon filter). In some examples, the activated carbon-based filter 25 can include an activated charcoal (e.g., an activated charcoal filter). In some aspects, the activated carbon-based filter 25 is configured to capture (and retain) gases (e.g., chemicals).

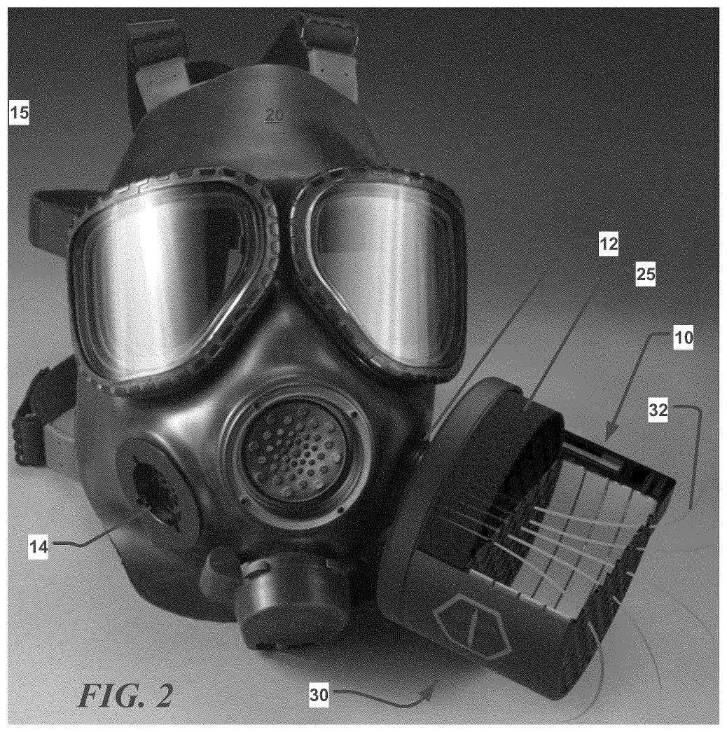

The hybrid filter 30 not only provides superior filtration advantages over a activated carbon-based filter 25, but it also provides a level of backup redundancy. For example, because the electro-ionic filter 10 is coupled in series with a activated carbon-based filter 25 well known in the art as illustrated in FIGS. 2 and 9, if the battery power for the electro-ionic filter 10 is used up in the field to the point that the electro-ionic filter 10 cannot fulfill its filtration responsibilities, the user can simply revert to the activated carbon-based filter 25 until the electro-ionic filter 10 can be recharged.

As can be understood from the preceding discussion and as will be further fleshed out in the following discussion, the hybrid filter 30 is advantageous for a number of reasons. First, the combined series filtration of the hybrid filter 30 provides the highest level of protective performance by employing both ionization and material-based filtration. The hybrid filter 30 is additionally advantageous in that is provides a fallback filtration capability. Specifically, as already stated above, the hybrid filter 30 can revert to material-based filtration when ionization is depowered. Thus, the hybrid filter 30 provides improved protection and failure safety over the prior art material-based filtration systems. Further, due to the embedded active electronic components of the hybrid filter 30, remote monitoring, control, sensing, and performance validation can be integrated into the design. Current material-based filtration systems lack such monitoring and sensing capabilities.

To begin a detailed discussion of the electro-ionic filter 10 forming part of the hybrid filter 30, reference is made to FIG. 1, which is a front perspective view of the overall filtration system 15. As shown in FIG. 1, the overall filtration system 15 includes a mask 20 and the hybrid filter 30 operatively connected to a first port 12 of the mask 20 in the same manner a standard 40 mm canister filter would be attached.

As those familiar with 40 mm canister filters will understand from FIG. 1, the hybrid filter 30 is substantially similar in size and configuration to common 40 mm canister filter that offer material-based filtration. As a result, should the need arise and should the mask support doing so, which is the case with the M40A1 Field Protective Gas Mask 20 depicted in FIG. 1 and other similar masks like the M50 Series Joint Service General Purpose Mask (JSGPM), a second hybrid filter 30 can be operatively connected to a second port 14 of the mask 20 in the same manner a second standard 40 mm canister filter would be attached. Thus, the hybrid filter 30 can be utilized in any application designed to receive the standard 40 mm canister filter. Of course, the hybrid filter 30 should not be limited to being of the same shape and size as the standard 40 mm filter as in other embodiments the hybrid filter 30 may come in other shapes, sizes and be configured to be interchanged with other types of material-based filters for use with other types of gas masks.

The discussion now turns to FIG. 2, which is the same view as FIG. 1, except the hybrid filter 30 has been sectioned to reveal some of its internal components. As indicated in FIG. 2, the hybrid filter 30 includes the electro-ionic filter 10 and the activated carbon-based filter 25. The activated carbon-based filter 25 can be any filter element formed of activated charcoal and, in some cases, can include interposed dense fiber layers.

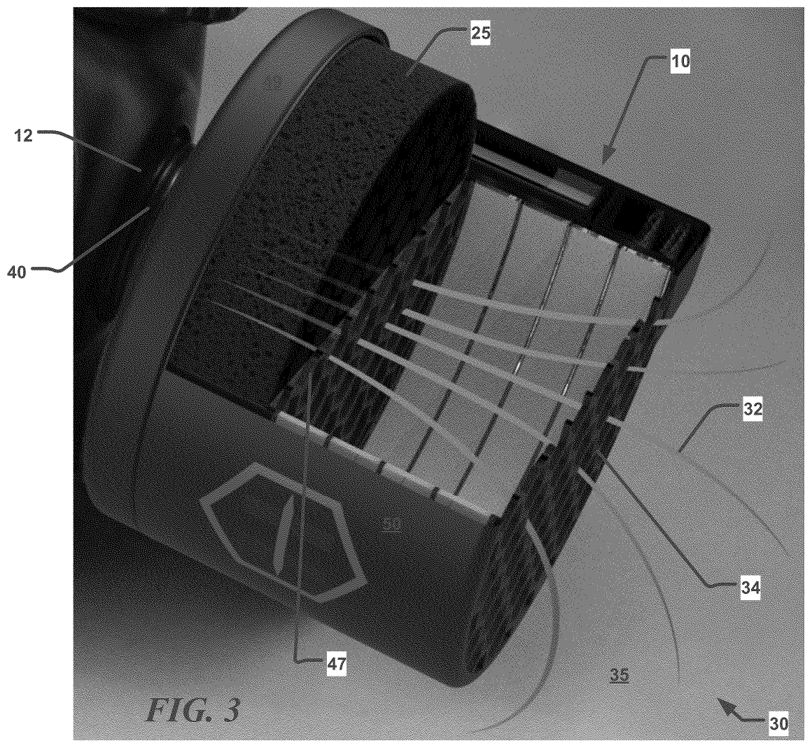

Referring now to FIG. 3, which is an enlarged view of the sectioned hybrid filter 30 from FIG. 2, the electro-ionic filter 10 and the activated carbon-based filter 25 are oriented in series with respect to the direction of the airflow through the hybrid filter 30, as can be understood from the airflow lines 32. Because of its superior filtration performance and its ability to neutralize contaminants, the electro-ionic filter 10 is positioned upstream in the airflow 32 relative to the activated carbon-based filter 25. In other words, the electro-ionic filter 10 is immediately adjacent an intake grille 34 of the hybrid filter 30, the intake grille 34 opening from the ambient surrounding environment 35 into the interior of the electro-ionic filter 10. The activated carbon-based filter 25 and the electro-ionic filter 10 can each define a longitudinal axis. In some embodiments, the airflow 32 through the hybrid filter 30 is substantially parallel to the longitudinal axis of the activated carbon-based filter 25, the longitudinal axis of the electro-ionic filter 10, or both. In some embodiments, the longitudinal axis of the activated carbon-based filter 25 is coaxial with the longitudinal axis of the electro-ionic filter 10.

Conversely, as depicted in FIG. 3, the activated carbon-based filter 25 is immediately adjacent an airflow exit 40 of the hybrid filter 30, the airflow exit 40 leading into the first port 12 of the mask 20. Due to the airflow 32 first traveling from the ambient surroundings 35 into the electro-ionic filter 10 and then into the activated carbon-based filter 25 before passing into the mask 20, the electro-ionic filter 10 filters out, and neutralizes, most of the contaminants (e.g., over 99% in some cases) before they can reach the activated carbon-based filter 25. Further, the ozone generated by the electro-ionic filter 10 neutralizes any contaminants trapped in the activated carbon-based filter 25. Finally, should the electro-ionic filter 10 cease operation for one reason or the other in the field, the activated carbon-based filter 25 provides a level of backup redundancy, the activated carbon-based filter 25 having essentially a full use life remaining when the electro-ionic filter 10 ceased functioning.

It should be noted that the airflow exits the hybrid filter 30 at the airflow exit 40 as the wearer of the mask 20 inhales. In some embodiments, such as when the hybrid filter 30 (and the mask 20 to which the hybrid filter 30 is coupled) is configured for bidirectional airflow, the airflow enters the hybrid filter 30 from the first port 12 of the mask 20 at the airflow exit 40 of the hybrid filter 30 when the wearer of the mask 20 exhales. Then, the airflow 32 flows through the hybrid filter 30 (e.g., through the activated carbon-based filter 25) and into the ambient surroundings 35.

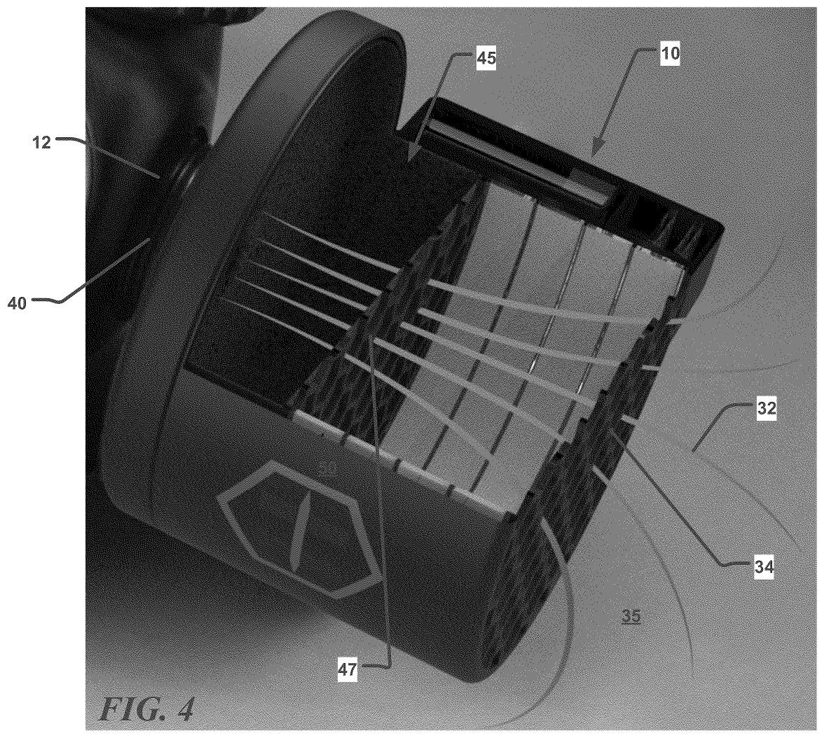

As can be understood from FIG. 4, which is the same view of the sectioned hybrid filter 30 as FIG. 3, except shown with the activated carbon-based filter 25 removed, the hybrid filter 30 can provide superior filtration and neutralization of contaminants even with the activated carbon-based filter 25 being absent from the hybrid filter 30. This ability to provide superior filtration is due to the electro-ionic filter 10 being the workhorse of the hybrid filter 30 and offering significantly superior filtration as compared to that offered by the activated carbon-based filter 25.

As shown in FIG. 4, removal of the activated carbon-based filter 25 reveals a chamber 45 in which the activated carbon-based filter 25 (not shown in FIG. 4) is received, the chamber 45 being on a downstream side of an inner grille 47 of the electro-ionic filter 10 and upstream of a housing cap 49 that forms a removable lid of a housing 50 of the hybrid filter 30. The activated carbon-based filter 25 (when present) and the electro-ionic filter 10 are disposed within the housing 50. In this manner, the housing 50 surrounds the activated carbon-based filter 25 and the electro-ionic filter 10.

In some embodiments, the filter employed with the overall filtration system 15 of FIG. 1 may simply include a mask 20 and the electro-ionic filter 10 of FIG. 4 operatively connected to the first port 12 of the mask 20. In other words, the hybrid filter 30 of FIG. 3 may be replaced by a dedicated electro-ionic filter 10 of FIG. 4, such a filter lacking any activated carbon-based filter 25. While such a solely electro-ionic filter 10 will not provide the backup redundancy of the hybrid filter 30, it will still provide exceptional filtration and neutralization of contaminants simply because of the filtration capabilities of the electro-ionic filter 10.

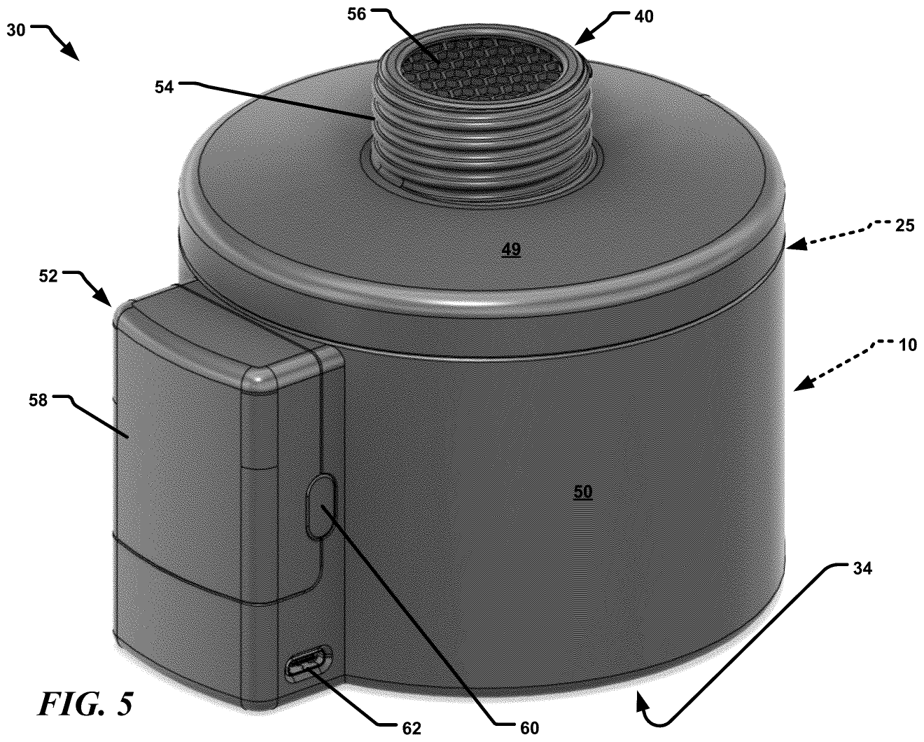

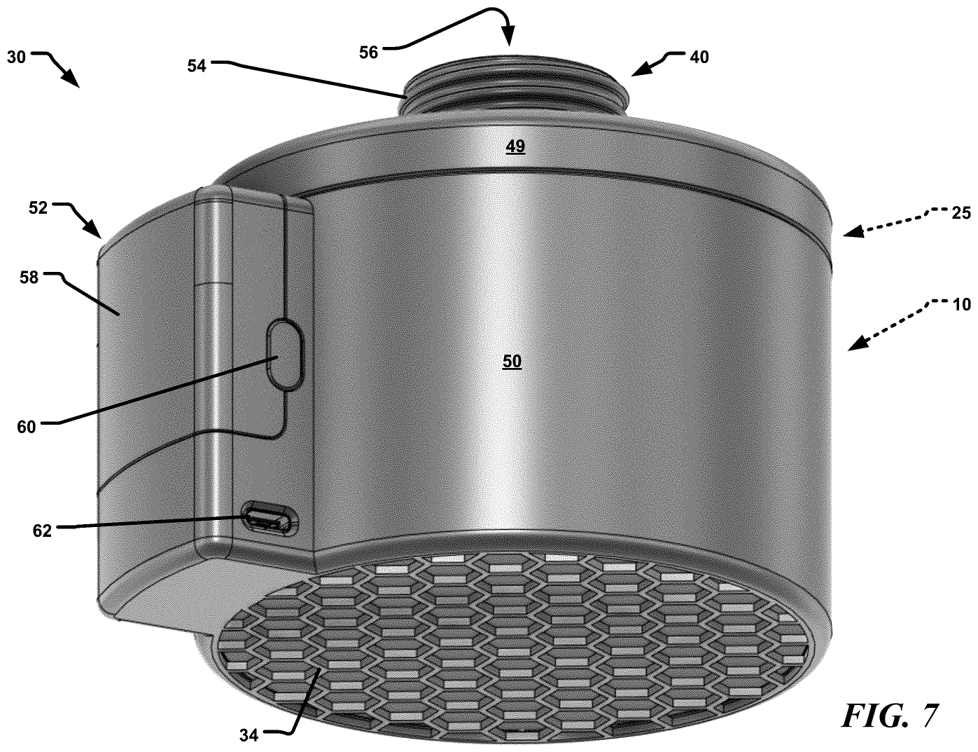

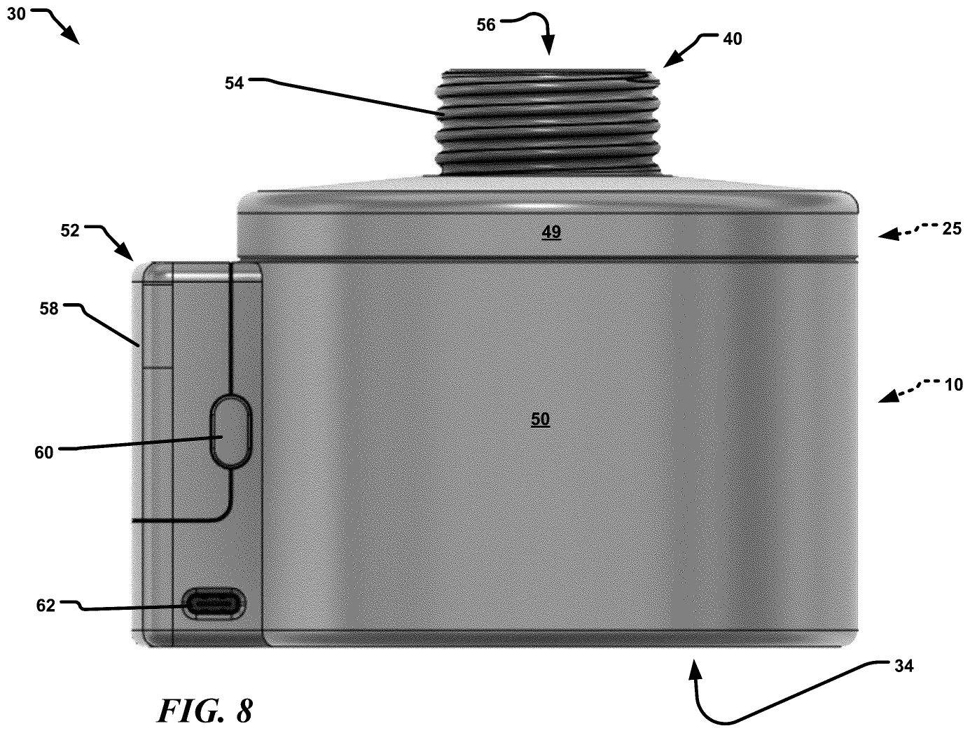

As depicted in FIGS. 5-8, which are various exterior views of the hybrid filter 30, the exterior of the hybrid filter 30 includes the housing 50 and an electronics box 52 mounted on the side of the housing 50. The housing 50 includes the housing cap 49 (also referred to as a removable lid) removably coupled to the rest of the housing 50 via common mechanical arrangement such as, for example, threads, bayonet lock, friction fit, etc.

The airflow exit 40 (also referred to as the airflow outlet) projects upwardly from the housing cap 49 and includes exterior threads 54 for threaded attachment to the first port 12 of the mask 20. The airflow exit 40 includes an exit grille 56 extending across the opening of the airflow exit 40.

The bottom of the housing 50 terminates in the intake grille 34. In one embodiment, the housing 50 (with its housing cap 49), the exit grille 56 and the intake grille 34 are all made of metal, such as, for example, aluminum, steel, copper, etc. In some embodiments, any one or more of the housing 50 (with its housing cap 49), the exit grille 56 and/or the intake grille 34 may be made of any of the aforementioned metals and/or a polymer coated with a conductive coating. Together these structural elements (housing 50, housing cap 49, exit grille 56, and intake grille 34) all combine to form a Faraday cage electrically insulated from, but surrounding and enclosing, the electrode components of the electro-ionic filter 10. The Faraday cage isolates the electrical environment produced by components of the electro-ionic filter 10 within the Faraday cage from the surrounding ambient environment 35 and the user of the mask 20, thereby protecting the user.

The housing 50, housing cap 49, exit grille 56, inner grille 47, intake grille 34, and/or a combination thereof can form a Faraday cage. For example, the inner grille 47 can be made of same or similar materials as the exit grille 56 and/or intake grille 34. In one embodiment, the housing 50, the intake grille 34, and the inner grille 47 can together form a Faraday cage that encapsulates the electro-ionic filter 10.

The intake grille 34, the inner grille 47, and the exit grille 56 each define a longitudinal axis. In some embodiments, the longitudinal axis of the intake grille 34 is colinear with the longitudinal axis of the exit grille 56. In some embodiments, the longitudinal axis of the intake grille 34 is colinear with the longitudinal axis of the inner grille 47. In some embodiments, the longitudinal axis of the inner grille 47 is coaxial with the longitudinal axis of the exit grille 56.

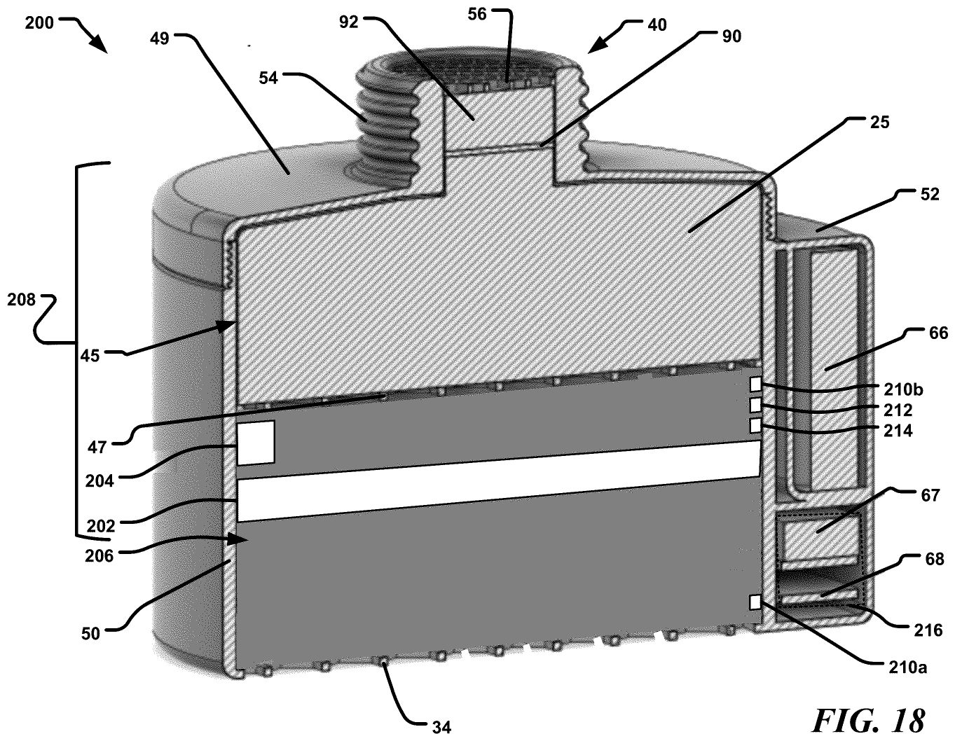

The electronics box 52 is coupled to the hybrid filter 30. In some embodiments, the electronics box 52 includes a computing device 216 (e.g., controller, microcontroller), as illustrated for example in FIGS. 18-20, disposed therein. In some embodiments, the electronics box 52 is mounted on a side of the housing 50 and includes a removable access lid 58, a power button 60, power cord receptacle 62, and a status indicator 64. The removable access lid 58 of the electronics box 52 leads to the electronic components (which can include the computing device, as illustrated in FIGS. 18-20) and battery located within the electronics box 52. The power button 60 is used to power on/off the electro-ionic filter 10, and in some embodiments control the level of electro-ionic filtration offered by the electro-ionic filter 10. The status indicator 64 may be in the form of a sound and/or light indicator for notifying the user of the operational state of the electro-ionic filter 10.

The power cord receptacle 62 may be in the form of a USB port or other type of power cord receptacle. The power cord receptacle 62 may be used for charging a rechargeable battery of the electro-ionic filter 10. The power cord receptacle 62 may also be used to upload programing into the electro-ionic filter 10 or downloading data therefrom. The port USB port may be in the form of a USB Type-C charging and data port.

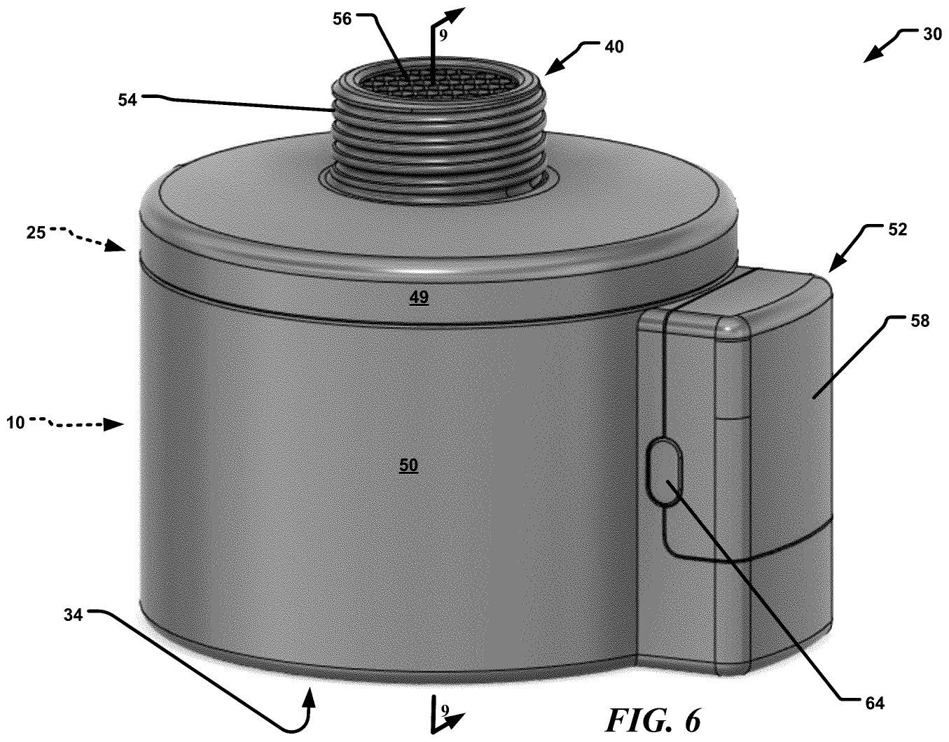

As shown in FIG. 9, which is a cross-sectional perspective view of the hybrid filter as taken along section line 9-9 in FIG. 6, the electronics box 52 includes a series of battery cells 66 and printed circuit boards (PCBs) 67, 68 containing the circuitry, memory and processor that runs the electro-ionic filter 10. In one embodiment, the battery cells 66 may be three LiPo cells that are rechargeable via power administered to the battery cells via the power cord receptable 62. In other embodiments, the battery cells 66 may be of other types and/or may be more or less than three cells. The battery pack may be removable.

As indicated in FIG. 9 working upward from the bottom intake grille 34, there is a first electrode 70 immediately upward from the intake grille 34. A first offset space 72 exists upwardly of the first electrode 70 and below a second electrode 74. A second offset space 76 exists upwardly of the second electrode 74 and below a third electrode 78. A third offset space 80 exists upwardly of the third electrode 78 and below a fourth electrode 82. A fourth offset space 84 exists upwardly of the fourth electrode 82 and below a fifth electrode 86. The fifth electrode 86 is immediately below the inner grille 47. The intake grille 34 and inner grille 47 form the upper and lower boundaries of the electrode region of the electro-ionic filter 10.

In one embodiment, each offset space 72, 76, 80 and 84 is between approximately 5 mm and approximately 15 mm with a concurrent operating voltage for the electrodes 70, 74, 78, 82, 86 of between approximately 2 kV and approximately 18 kV. In another embodiment each offset space 72, 76, 80 and 84 is between approximately 7 mm and approximately 13 mm with a concurrent operating voltage for the electrodes 70, 74, 78, 82, 86 of between approximately 5 kV and approximately 15 kV. Finally, in another embodiment, each offset space 72, 76, 80 and 84 is between approximately 9 mm and approximately 11 mm with a concurrent operating voltage for the electrodes 70, 74, 78, 82, 86 of between approximately 8 kV and approximately 12 kV.

As can be understood from FIG. 9, each offset space 72, 76, 80 and 84 is established between the various vertically offset pairs of electrodes 70, 74, 78, 82, 86 by cylindrical spacer sections 88A, 88B, 88C, 88D. The cylindrical spacer sections are interposed between each vertically offset pair of electrodes in a stacked alternating arrangement of electrodes and cylindrical spacer sections. Each cylindrical spacer section 88A, 88B, 88C, 88D has an outer circumferential cylindrical surface that is electrically insulative and in abutting coextensive contact with an equally sized portion of the inner circumferential cylindrical surface of the housing 50. Thus, although each cylindrical spacer section 88A, 88B, 88C, 88D is in abutting coextensive contact with the inner circumferential cylindrical surface of the housing 50, each cylindrical spacer section 88A, 88B, 88C, 88D and the associated electrode 70, 74, 78, 82, 86 is electrically isolated from the housing 50, which acts as part of a Faraday cage as described above.

In one embodiment, the cylindrical spacer sections 88A, 88B, 88C, 88D and associated electrodes 70, 74, 78, 82, 86 form a unitary structure that can be removed from with the housing 50 as a single piece. In other embodiments, the cylindrical spacer sections 88A, 88B, 88C, 88D and associated electrodes 70, 74, 78, 82, 86 are individual pieces or an individual electrode and individual cylindrical spacer section may be joined as a unitary structure to form a module that can be removed from within the housing 50 for cleaning, servicing or replacement.

In configurations where a cylindrical space section and its associated electrode is a unitary structure forming a module that can be removed and replaced as a unit, such a unit may have electrical contacts that interface and contact with paired electrical contacts within the housing 50. Such paired contacts may be in the form of pogo pins, contact pads, or etc. to allow the unit to be effortlessly removed and reinstalled within the housing and establish electrical conductivity between the electrode of the unit and the rest of the electro-ionic filter.

Moving upward from the first electrode 70 along the stack of electrodes 70, 74, 78, 82, 86 the polarity of one electrode will alternate back and forth such that the first electrode 70 will be one polarity and the second electrode 74 will be the opposite polarity, and so forth moving upward. Thus, in one embodiment, the first electrode 70, the third electrode 78, and the fifth electrode 86 will be positive electrodes, and the second electrode 74 and fourth electrode 82 will be negative electrodes. Such a five electrode arrangement can be called a positive-negative-positive-negative-positive (P-N-P-N-P) arrangement. With the subtraction of two electrodes for a total of three electrodes, the arrangement would be a P-N-P arrangement. With the addition of two more electrodes for a total of seven electrodes, the arrangement would be a P-N-P-N-P-N-P arrangement. An even great number of electrodes can be provided, only being limited by size, weight and power consumption considerations.

Electrode arrangements that begin and end with a positive electrode (e.g., a P-N-P, a P-N-P-N-P arrangement, a P-N-P-N-P-N-P arrangement, etc.) are the most efficient and preferred arrangements. However, it is possible to have electrode arrangements that begin and end with a negative electrode (e.g., a N-P-N, a N-P-N-P-N arrangement, a N-P-N-P-N-P-N arrangement, etc.).

Within the electro-ionic filter 10, each positive electrode acts as a positive collector, and each negative electrode acts as a negative emitter. Regardless of the number of electrodes 70, 74, 78, 82, 86 employed in forming the electro-ionic filter 10, in the embodiment depicted in FIG. 9, the electrostatic precipitation filter arrangement provided by the spaced-apart parallel electrodes 70, 74, 78, 82, 86 results in a linear parallel dispersive geometry for entrapping and neutralizing toxin/contaminant particles, regardless of whether those particles are in the form of airborne bio pathogens, a biohazardous mist and/or gaseous chemicals.

Whether the electro-ionic filter 10 is arranged to have a single layer device with a negative electrode sandwiched between two positive electrodes (i.e., P-N-P electrode arrangement), or the electro-ionic filter 10 is arranged to have two layers (i.e., P-N-P-N-P) or more layers (e.g., P-N-P-N-P-N-P; P-N-P-N-P-N-P-N-P, etc.), then the series of electrode grids 70, 74, 78, 82, 86 will be arranged such that the series of stacked spaced-apart electrode grids 70, 74, 78, 82, 86 will start on the bottom of the stack with a positive (P) electrode 70 and end at the top with another positive (P) electrode 86. In some embodiments, such positive (P) starting and ending stacks allows the positive electrodes 70, 86 to function as part of a floating ground Faraday cage that encapsulates the high voltage negative (N) electrode grids 74, 82 as well as imparting a bidirectional particle capture efficacy in those embodiments where the hybrid filter 30 and mask 20 are set up for bidirectional flow. While the example mask 20 depicted in FIGS. 1 and 2 is typically set up for unidirectional airflow, other masks can be set up for bidirectional airflow, and the hybrid filter 30 and its electro-ionic filter 10 can be set up to work with either type of airflow.

The bidirectional airflow capability may make the mask less comfortable to the user due to having to inhale and exhale through the hybrid filter 30. However, it does provide the benefit that the exhaled air from the user is filtered/neutralized. As a result, if the user is infected or a carrier of an airborne infectious disease, those people around the user who are not using a mask have a much-reduced risk of getting infected by the user of the bidirectional mask as compared to the risk presented by a user of a unidirectional mask.

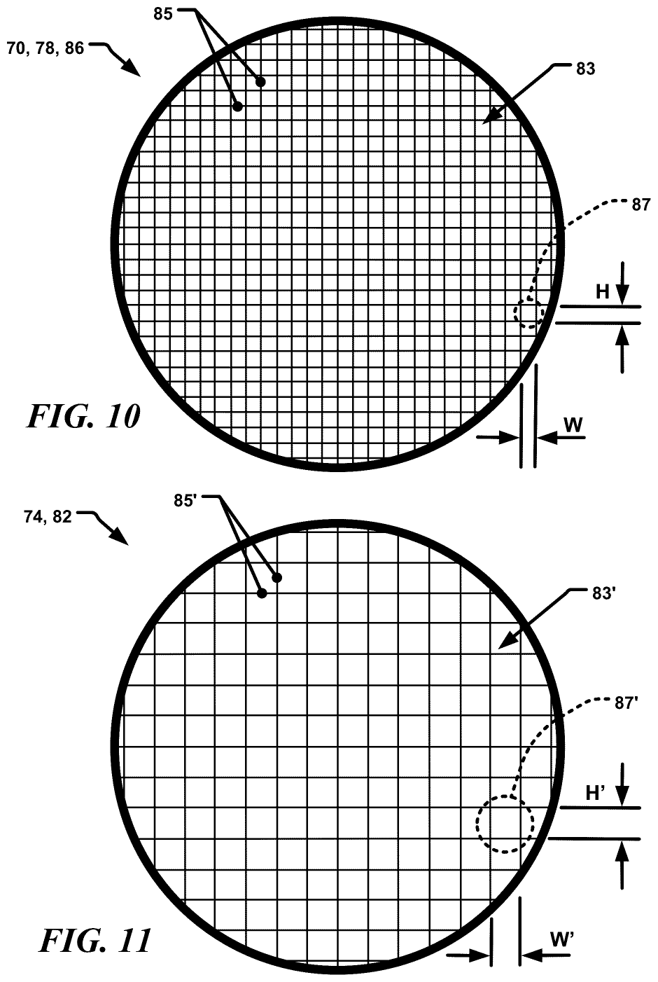

FIG. 10 is a top plan view of an electrode configuration 83 representative of that employed for the positive electrodes 70, 78, 86, and FIG. 11 is a top plan view of an electrode configuration 83′ representative of that employed for the negative electrodes 74, 82. As can be understood from FIGS. 10 and 11, each electrode 70, 74, 78, 82, 86, regardless of its polarity, is in the form of an open grid or grille 83, 83′ extending across the chamber of the electro-ionic filter 10 perpendicular to the side walls of the housing 50, as depicted in FIG. 9. The open grid or grille configuration 83, 83′ of the electrodes allows airflow to pass through the series of electrodes 70, 74, 78, 82, 86 perpendicular to the electrodes 70, 74, 78, 82, 86, as shown in FIGS. 2-4.

The series of vertically offset electrode grids 70, 74, 78, 82, 86 will be of variable metal content with alternating charge sequences. In one embodiment as can be understood via a comparison of FIG. 10 and FIG. 11, the grid/grille configuration 83′ of each negative emitter electrode 74, 82 will have more total open area for airflow as compared to the grid/grille configuration 83 of each positive collector electrode 70, 78, 86 that will have less open area for airflow. Also, the grid/grille configuration 83′ of each negative emitter electrode 74, 82 will have less metal making up the thin metal components 85′ of the metal grille/grid 83′ forming the emitter surfaces of the negative emitter electrode 74, 82 as compared to the thin metal components 85 of the metal grille/grid 83 forming the collector surfaces of the grid/grille configuration 83 of each positive collector electrode 70, 78, 86.

Optimizing the capabilities of the electro-ionic filter 10 is a balancing exercise between the filtration/neutralization capabilities of the electro-ionic filter 10 and its practical size/weight and ease of breathing through the electro-ionic filter 10. For example, there are several ways to enhance particle capture/neutralization in the parallel electrode configuration employed in the electro-ionic filter 10. One way is to make the collector electrodes 70, 78, 86 with more metal surface and less airspace but that in turn will offer increased resistance to airflow and degrade the respiratory comfort of the electro-ionic filter 10.

The following grid dimensions and offset distances between the electrode grids 70, 74, 78, 82, 86 provide example embodiments where the electro-ionic filter 10 has been optimized with respect to the desirable balance between the filtering/neutralizing capabilities of the electro-ionic filter 10 and its size, weight and breathability. In other words, the configurations recited below provides electrode grids and arrangements that offer sufficient conductive surface for the collector electrodes 70, 78, 86 while still offering low airflow resistance. Further, the configurations recited below also optimize the fixed distances 72, 76, 80, 84 between the series of vertically offset electrode grids 70, 74, 78, 82, 86, plus the optimal voltage to drive the electric field associated therewith.

In one embodiment as illustrated in FIG. 10, the positive grid/grille configuration 83 of each positive collector electrode 70, 78, 86 will be made of thin components 85 such as wire 85 made of steel, silver, platinum, carbon, iridium coated materials, tantalum coated materials, etc., or other appropriate electrically conductive metals, alloys or coatings over a supportive substrate.

In one embodiment, the positive collector wire 85 will be much larger in diameter than the negative emitter wire 85′ discussed below. For example, the positive collector wire 85 may not even be truly a wire, but could be more like a rod. Regardless of how it is classified, the positive collector wire 85 may have a diameter of between approximately 0.01 mm and approximately 5 mm. In another embodiment, the wire 85 will have a diameter of between approximately 0.01 mm and approximately 3 mm. In yet another embodiment, the wire 85 will have a diameter of between approximately 0.01 mm and approximately 2 mm. Finally, in one embodiment, the wire 85 will have a diameter of between approximately 0.5 mm and approximately 1 mm.

In instances where wire intersections occur, these intersections ideally should be flat intersections such that one wire 85 does not pass over the other wire 85 in a stacked configuration. This flat intersection will result in consistent offset spacing 72, 76, 80, 84 across the positive grid/grille configuration 83 relative to the immediately adjacent negative grid/grille configuration 83′, thereby avoiding inconsistencies in the gaps 72, 76, 80, 84 between the positive and negative electrodes that could lead to shorting.

As illustrated in FIG. 10, in one embodiment, the positive grid/grille configuration 83 of each positive collector electrode 70, 78, 86 may be rectangular, or even square, with each wire 85 offset from its immediately adjacent neighbor wire 85 such that a rectangular open area 87 is created between the wires 85. The cumulative free area of all the open areas 87 through which air may pass through the positive grid/grille configuration 83 of each positive collector electrode 70, 78, 86 relative to the cumulative metal surface provided by all of the wires 85 on which particles may be collected due to ionization and electrostatic precipitation is a ratio of between approximately 100/1 and approximately 50/50 cumulative metal surface/cumulative fee area.

In yet other embodiments, the positive electrode grid 83 can be configured such that the open area 87 is of other shapes such as, for example, oval, circular, triangular, etc. and has other dimensions.

Further, in some embodiments, the wires 85 of the positive electrode grid/grille configuration 83 could all simply run parallel in the same direction such that the open areas 87 end up being simply parallel strips of open space extending wall-to-wall across the associated cylindrical spacer sections 88A, 88B, 88C, 88D and being generally evenly spaced, or not.

In other embodiments, the wires 85 of the positive electrode grid 83 may be distributed within the confines of the associated cylindrical spacer sections 88A, 88B, 88C, 88D in a random direction and spacing arrangement.

In all these alternative embodiments, the cumulative free area of all the open areas 87 through which air may pass through the positive grid/grille configuration 83 of each positive collector electrode 70, 78, 86 relative to the cumulative metal surface provided by all of the wires 85 on which particles may be collected due to ionization and electrostatic precipitation is a ratio of between approximately 100/1 and approximately 50/50 cumulative metal surface/cumulative fee area.

In some embodiments, the positive grid/grille configuration 83 of each positive collector electrode 70, 78, 86 will be a solid sheet or plate extending wall-to-wall across the cylindrical spacer sections 88A, 88B, 88C, 88D and having a series of generally evenly spaced-apart holes 87 passing therethrough and formed therein via mechanical machining processes, laser cutting, etc. The holes 87 of the positive grid/grille configuration 83 may be oval, circular, square, rectangular, triangular, etc., or a combination of these shapes. The plate may be made of steel, silver, platinum, carbon, iridium coated materials, tantalum coated materials, etc., or other appropriate electrically conductive metals, alloys or coatings over a supportive substrate. The cumulative free area of all the spaced-apart holes 87 through which air may pass through the positive grid/grille configuration 83 of each positive collector electrode 70, 78, 86 relative to the cumulative metal surface provided by the rest of the plate on which particles may be collected due to ionization and electrostatic precipitation is a ratio of between approximately 100/1 and approximately 50/50 cumulative metal surface/cumulative fee area.

In one embodiment as depicted in FIG. 11, the negative grid/grille configuration 83′ of each negative emitter electrode 74, 82 will be made of thin metal components 85′ such as wire 85′ made of steel, silver, platinum, carbon, iridium coated materials, tantalum coated materials, etc., or other appropriate electrically conductive metals, alloys or coatings over a supportive substrate.

In one embodiment, the negative emitter wire 85′ will be much smaller in diameter than the positive collector wire 85 discussed above. For example, the negative emitter wire 85′ may have a diameter of between approximately 0.01 mm and approximately 1 mm. In another embodiment, the wire 85′ will have a diameter of between approximately 0.01 mm and approximately 0.5 mm. In yet another embodiment, the wire 85′ will have a diameter of between approximately 0.01 mm and approximately 0.2 mm. Finally, in one embodiment, the wire 85′ will have a diameter of between approximately 0.01 mm and approximately 0.1 mm.

In instances where wire intersections occur, these intersections ideally should be flat intersections such that one wire 85′ does not pass over the other wire 85′ in a stacked configuration. This flat intersection will result in consistent offset spacing 72, 76, 80, 84 across the negative grid/grille configuration 83′ relative to the immediately adjacent positive grid/grille configuration 83, thereby avoiding inconsistencies in the gaps 72, 76, 80, 84 between the positive and negative electrodes that could lead to shorting.

As illustrated in FIG. 11, the negative grid/grille configuration 83′ of each negative emitter electrode 74, 82 may be rectangular, or even square, with each wire 85′ offset from its immediately adjacent neighbor wire 85′ such that a rectangular open area 87′ is created between the wires 85′. The cumulative free area of all the open areas 87′ through which air may pass through the negative grid/grille configuration 83′ of each negative emitter electrode 74, 82 relative to the cumulative metal surface provided by all of the wires 85′ to serve as an emitter to charge particles in the airflow as part of the ionization and electrostatic precipitation process is a ratio of between approximately 1/100 and approximately 50/50 cumulative metal surface/cumulative fee area.

As can be understood from a comparison of electrodes of FIGS. 10 and 11, the number of the wires 85 for the positive collector electrodes 70, 78, 86 is much greater than the number of the wires 85′ of the negative emitter electrodes 74, 82. As a result, the cumulative amount of open areas 87 of the positive collector electrodes 70, 78, 86 is going to be less than the cumulative amount of open areas 87′ of the negative emitter electrodes 74, 82. This can be understood by comparing the height H, width W and general size of the open spaces 87 of the positive collector electrodes 70, 78, 86 as compared to the height H′, width W′ and general size of the open spaces 87′ of the negative emitter electrodes 74, 82.

In yet other embodiments, the negative electrode grid/grille configuration 83′ can be configured such that the open area 87′ is of other shapes such as, for example, oval, circular, triangular, etc. and has other dimensions. Further, in some embodiments, the wires 85′ of the negative electrode grid/grille configuration 83′ could all simply run parallel in the same direction such that the open areas 87′ end up being simply parallel strips of open space extending wall-to-wall across the associated cylindrical spacer sections 88A, 88B, 88C, 88D and being generally evenly spaced, or not.

In other embodiments, the wires 85′ of the negative electrode grid/grille configuration 83′ may be distributed within the confines of the associated cylindrical spacer sections 88A, 88B, 88C, 88D in a random direction and spacing arrangement.

In some embodiments, the negative electrode grid/grille configuration 83′ may not really qualify as a grille configuration but will be called such for purposes of the discussion. In such a so-called “grille” configuration, the grille configuration 83′ will simply be a small number of wires 85 (e.g., one, two or three wires 85′) spaced-apart from each other, extending across the associated cylindrical spacer sections 88A, 88B, 88C, 88D, and serving as emitters of the negative electrode 74, 82.

In all these alternative embodiments of the negative electrode grid/grille configuration 83′, the cumulative free area of all the open areas 87′ through which air may pass through the grid/grille configuration 83′ of each negative emitter electrode 74, 86 relative to the cumulative metal surface provided by all of the wires 85′ serving as an emitter surface for charging particles in the airflow through the negative electrode grid 83′ as part of the ionization and electrostatic precipitation process is a ratio of between approximately 1/100 and approximately 50/50 cumulative metal surface/cumulative fee area.

Ultimately, in some embodiments, the cumulative free areas through which air may flow through the series of positive and negative electrodes 70, 74, 78, 82, 86 should be such that the air resistance presented to a user respirating through the series of electrodes will not be uncomfortable or excessive at respiratory rates of between approximately 5 liters/minute and 85 liters/minute. In some embodiments, the total airflow resistance presented to a user of the overall filtration system 15 by respiration airflow extending across the hybrid filter 30 from the intake grille 34 and across the electrodes 70, 74, 78, 82, 86, the elements (e.g., activated carbon-based filter 25, particulate filter 90, ozone scrubber 92) and finally out the exit grille 56 will be less than that presented by a N95 mask. In other embodiments, the airflow resistance along such a path may be less than 340 Pascals at 85 liters/minute airflow. In other embodiments, the airflow resistance along such a path may be less than 200 Pascals at 85 liters/minute airflow.

Generally speaking, for purposes of helping one understand the operation of the electro-ionic filter 10, one could draw some similarities of the stacked offset electrode arrangement of the electro-ionic filter 10 to the functional design of a semiconductor device such as a P-N-P transistor where its performance is achieved through semiconductor doping. Such doping creates current carriers within the relatively non-conductive silicon wafer. In the case of some of the embodiments of the stacked offset electrode arrangement of the electro-ionic filter 10 disclosed herein, the electrode grid selection and offset space between such electrode grids allows the flowing air to become the semiconductor because its particles become the charge carriers ultimately trapped by the P electrode collectors. Air by itself is relatively non-conductive unless it is contaminated by particles. Such particles become the current carriers not unlike the doping within a solid state transistor design. The airborne bio pathogens flowing between the P-N-P electrode grid arrangements become the charge carriers of the current between the negative emitter electrode grid and the positive collector electrode grids.

In embodiments of the electro-ionic filter 10 having a P-N-P grid arrangement employing any of the electrode grid and offset dimensions described above or other electrode grid and offset dimensions not listed above, such embodiments can be further adjusted for optimizing filtration/neutralizing performance efficacy at the cost of a slight increase in size.

In one embodiment, each P-N-P set of spaced-apart electrode grids can be considered an electrode module. As illustrated in FIG. 9, each electrode grid offset spacing 72, 76, 80, 84 between any pair of electrode grids (e.g., 70 and 74, 74 and 78, 78 and 82, 82 and 86) may be such that the perpendicular distance between the parallel pair of electrode grids is approximately 1.5 cm+/−. 8 cm with and an applied voltage range of approximately 1 KV to approximately 8 KV. In one embodiment, as can be understood from FIG. 9, an electrode grid offset spacing 72, 76, 80, 84 of approximately 0.8 cm to approximately 1.6 cm may be employed with an applied voltage range of approximately 1 KV to approximately 8 KV.

Particle rejection in the parallel grid design can be improved by multiplying the number of P-N-P modules employed in the electro-ionic filter 10. Specifically, the number of P-N-P modules can be changed from one such module to two or more modules. Where two P-N-P modules are employed, the electrode stack with take the form of a P-N-P-N-P electrode stack. This double module arrangement will double the particle capture efficacy as compared to the single P-N-P module, all at the expense of making the electrode grid higher by, for example, approximately 4 cm to approximately 5 cm.

Adding yet another P-N-P module to the electro-ionic filter 10 to employ three P-N-P modules and a resulting P-N-P-N-P-N-P electrode stack will further increase the efficacy of the filtering/neutralizing provided by the electro-ionic filter 10 to that of three times the efficacy offered by a single P-N-P module, all at the expense of increased weight and size and a decrease in breathing comfort. As with the embodiments discussed above, the positive (P) electrodes on the top and bottom of the stack of electrodes serve as end portions of a faraday caging that encompassing the electro-ionic filter 10.

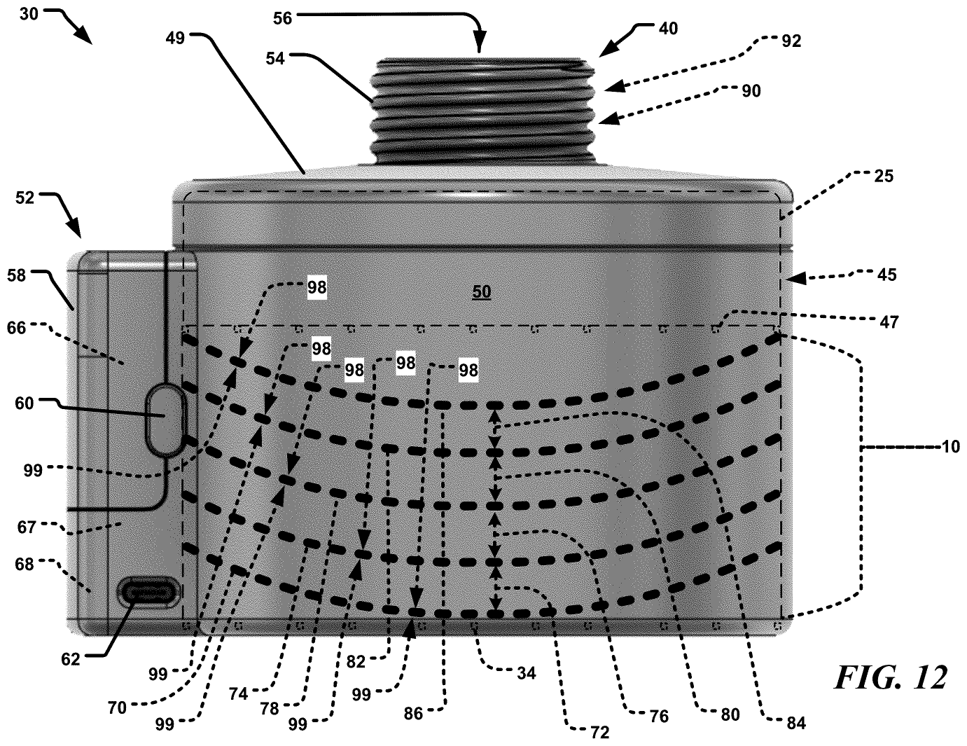

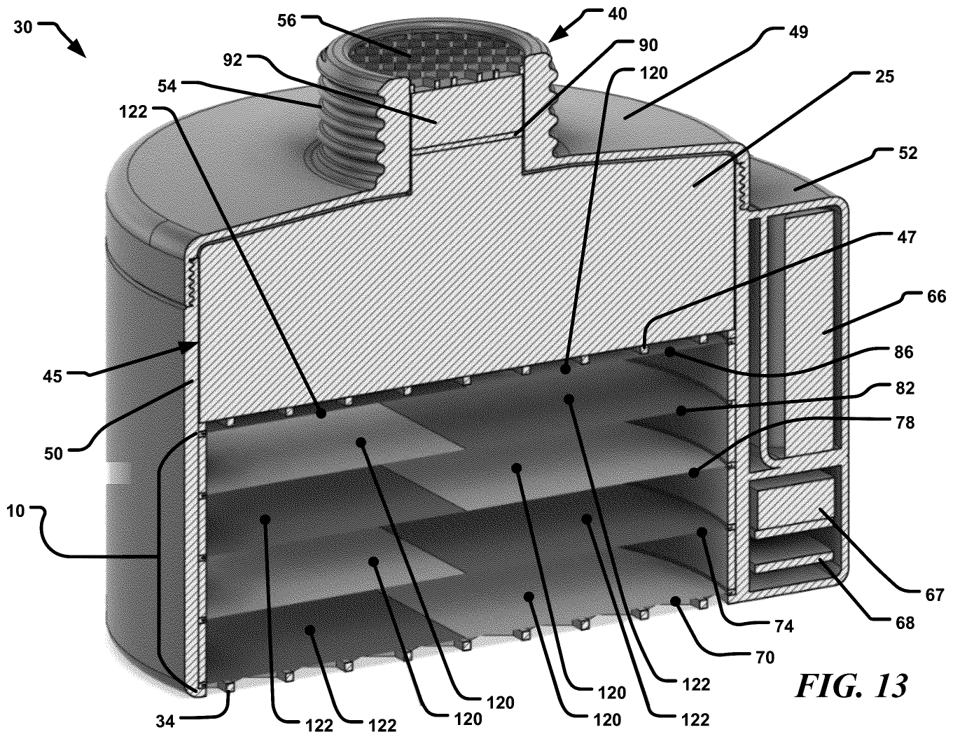

As can be understood from FIGS. 2-4 and 9 and as discussed above, in some embodiments, each electrode 70, 74, 78, 82, 86 of the spaced-apart stack of electrodes forming the electro-ionic filter 10 is flat or planar across its extent. However, in other embodiments, as depicted in FIG. 12, each electrode 70, 74, 78, 82, 86 is equally curved in all radial directions such that the electrode 70, 74, 78, 82, 86 is in the form of a bowl that is concave on its upward surface and convex on its downward surface. In other words, each electrode 70, 74, 78, 82, 86 has a concave upper surface 98 and a convex bottom surface 99. These bowl-like structures are then stacked much like a set of bowls. Due to being so stacked and the fact that the concave upper surface 98 and convex bottom surface 99 have the same curvature, the concave surface 98 of an electrode is equally spaced apart from the convex surface 99 of the electrode immediately above a consistent distance 72, 76, 80, 84 at all points on the concave surface. Utilizing the curved electrode arrangement as depicted in FIG. 12 provides a greater area to serve as a collection surface for the accumulation of charged particles in the airflow than the area provided by flat or planar electrodes as shown in FIG. 9.

While the embodiment of FIG. 12 utilizes curved electrodes with concave upper surfaces 98 and convex bottom surfaces 99, in other embodiments the reverse arrangement can be the case with convex upper surfaces and concave bottom surfaces.

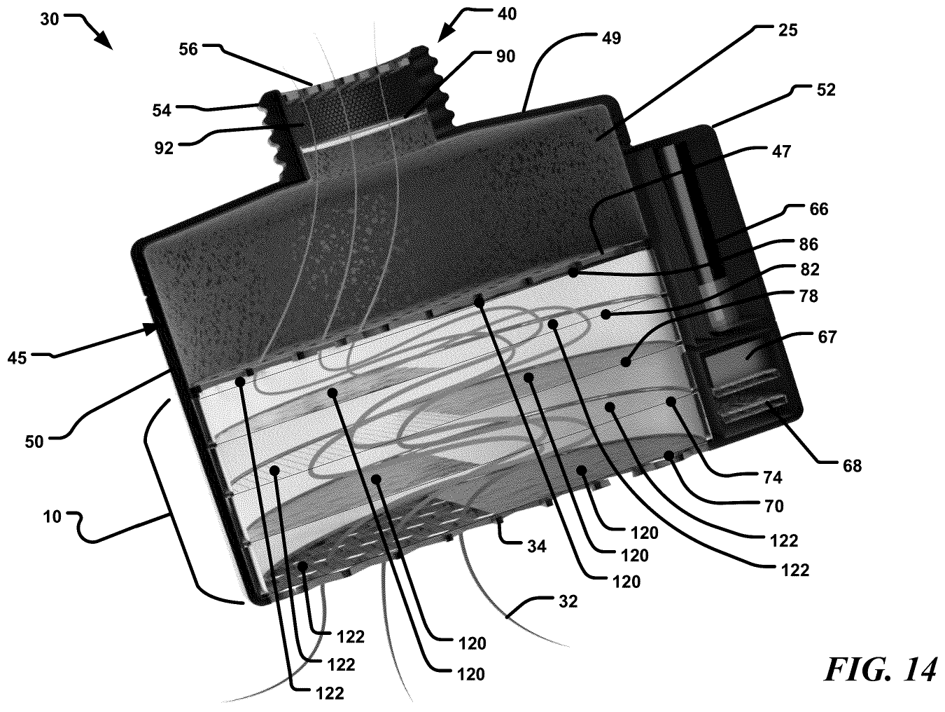

In some versions of the embodiments depicted in FIGS. 9 and 12, particle capture efficacy can be improved by modifying the airflow pathway through the stack of electrodes forming the electro-ionic filter 10 so as to prolong the flow contact with the positive collector electrodes 70, 78, 86 and move the air more perpendicular to the electric field. Such a diverted-airflow embodiment is depicted in FIGS. 13 and 14, which are cross-sectional views across the hybrid filter 30 as if taken along section line 9-9 in FIG. 6.

As illustrated in FIG. 13, the electro-ionic filter 10 incorporates an alternating conductive occlusion 120 of half of each positive collector electrode 70, 78, 86. As shown in FIG. 14, the conductive occlusions 120 result in a circuitous routing of the airflow through the electrode stack forming the electro-ionic filter 10, as can be understood from the airflow lines 32. As a result of the circuitous routing of the airflow 32, air hits the occluding surface 120 and has to flow sideways to the occlusion 120 of the positive collector electrode so as to pass through the non-occluded portion 122 of the positive collector electrode 70, 78, 86. The circuitous routing of airflow 32 provides for greater particle capture due to prolonged collector exposure, which allows for operational lower voltages and lower ozone by-product generation.

Judicious use of both the number of electrodes 70, 74, 78, 82, 86 placed in series to form the electrode stack of the electro-ionic filter 10 and the circuitous routing of airflow 32 through the electrode stack can improve the electrostatic precipitation (e.g., particle rejection) of the electro-ionic filter 10 at the cost of a slight increase in the size of the electrodes 70, 74, 78, 82, 86 and a slight increase in overall air resistance through the electro-ionic filter 10. This slight increase in size and resistance is as compared to the size and shape of the non-circuitous airflow embodiment of FIGS. 2-4 and 9.

As illustrated in FIGS. 9, 12 and 13, each embodiment of the hybrid filter 30 includes the integration of the electro-ionic filter 10 with material-based filters, such as the activated carbon-based filter 25 (e.g., activated charcoal filter), which acts as a capture layer for gases. The ionization generated by the electrode grids of the electro-ionic filter 10 captures particles and aerosols in such a manner so as to greatly expand the bandwidth of protection compared to that of simply the activated carbon-based filter 25, both in terms of the spectrum of protection against various agents but also in terms of the time during which such protection is functional and operational within a hostile environment.

The hybrid filter 30 can offer safe respiratory air in many environments and can readily be coupled with other supportive devices such as oxygen concentrators, closed circuit systems, partial recirculating systems, and power assisted filtration devices. Various configurations are possible with a variety of operational points with respect to particle rejection, depending on the specific industry need.

As studies are conducted regarding how the electro-ionic filter 10 performs with various agents under varying conditions, the electro-ionic filter 10 may be “tuned” to the specific environmental need. This “tuning” will be achieved by varying a number of design parameters regarding the components of the electro-ionic filter 10, namely, aspects of the electrodes 70, 74, 78, 82, 86 such as their associated geometry (radial or parallel) and size, the associated offset gap 72, 76, 80, 84, the design of the negative emitter electrode 74, 82, material choices for performance and oxidation resistance, operational voltages, current settings and modulation.

In some embodiments, the electro-ionic filter 10 can be integrated with network functionality, performance monitoring, and integration with embedded sensors and alarms. For example, integrated sensor technologies can sense characteristics of any captured toxin, chemical or otherwise.

As illustrated in FIG. 9, immediately upward of the inner grille 47 is the chamber 45 in which the activated carbon-based filter 25 is received. This chamber 45 is bounded on the sides by the inner cylindrical circumferential surface of the housing 50 and extends upward into the housing cap 49 and a neck of the airflow exit 40. The housing cap 49 can be removed to install or replace the element(s) or filter(s) (e.g., activated carbon-based filter 25, particulate filter 90 and ozone scrubber 92) contained in the chamber 45. The lower portion of the chamber 45 may be occupied by an activated carbon-based filter 25, above which may be located a particulate filter 90 (e.g., KN95 style material-based filter, N95 style material-based filter), all of which may be topped off with a ozone scrubber 92 (e.g., manganese oxide material-based filter, manganese oxide filter). Above the ozone scrubber 92 is the exit grille 56 extending across the opening of the airflow exit 40.

It should be noted here that the particulate filter 90 can include an N95 style filter (e.g., an N95 filter), an FFFP2 style filter (e.g., an FFFP2 filter), a KN95 style filter (e.g., a KN95 filter), a P2 style filter (e.g., a P2 filter), a DS2 style filter (e.g., a DS2 filter), a PFF2 style filter (e.g., a PFF2 filter), an equivalent filter, or a combination thereof. In some embodiments, the particulate filter 90 can include a high-efficiency particulate air (HEPA) filter or an equivalent filter. In some aspects, the particulate filter 90 is configured to capture (and retain) biological agents.

It should also be noted here that the ozone scrubber 92 include a metallic oxide (e.g., manganese oxide, copper oxide, cobalt oxide). In some aspects, the ozone scrubber 92 functions as a catalytic degrader. In some embodiments, the ozone scrubber 92 includes manganese oxide (e.g., a manganese oxide filter), cobalt oxide (e.g., a cobalt oxide filter), or a combination thereof. In some examples, the ozone scrubber 92 is a grid that includes manganese oxide.

The activated carbon-based filter 25 serves as a backup to the electro-ionic filter 10 to remove and neutralize toxins. The particulate filter 90 also serves as a backup to the electro-ionic filter 10 and to remove contaminants that escape the activated carbon-based filter 25. Finally, the ozone scrubber 92 serves to scrub ozone generated by electro-ionic filter 10 in the course of its operation. In one embodiment, the ozone scrubber 92 may be made of a grid with impregnated Manganese oxide that serves as a catalytic degrader (scrubber) of ozone that is incidentally generated by the ionization functionality of the electro-ionic filter 10.

Ozone is generated by the ionization function of the electro-ionic filter 10 (e.g., high voltage emitter-collector) in performing its filtering and neutralizing functions. This ozone may now be used to sanitize the bio-pathogens and oxidize biohazardous mists retained in the material-based filter elements (e.g., the activated carbon-based filter 25 and particulate filter 90) of the hybrid filter 30, thereby reactivating (or otherwise regenerating the absorptive capacity of) the activated charcoal filter and/or KN95 filter after having been saturated with bound chemical toxins and/or pathogens. This feature is a beneficial aspect of the hybrid filter 30 that uses and integrates the existing filtration elements (e.g., activated carbon-based filter 25, particulate filter 90) with the highly effective electro-ionic filter 10. In some aspects, the ozone neutralizes organophosphates (e.g., nerve gas). In some aspects, the ozone degrades (e.g., via oxidative degradation) the organic compounds retained by the material-based filter elements (e.g., the activated carbon-based filter 25). In some aspects, the ozone regenerates (e.g., via controlled oxidation at ambient temperature) the absorptive capacity of the material-based filter elements (e.g., the activated carbon-based filter 25).

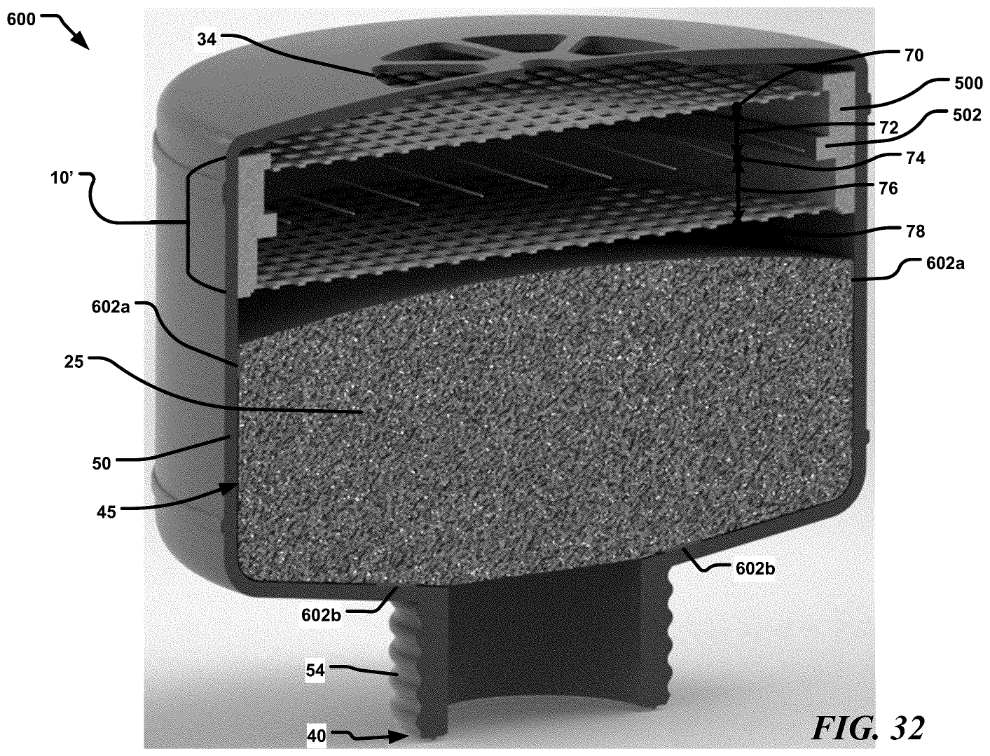

In some embodiments, one or more gaskets (not shown in FIG. 9) can be included at the joint between the activated carbon-based filter 25 and the chamber 45 (e.g., housing cap 49). For example, the one or more gaskets can be the same or similar as the gaskets 602 (e.g., gasket 602a, gasket 602b), which are illustrated for example in the cross-sectional view in FIG. 32. When present, the gasket impedes, or otherwise prevents, air from flowing through the joint between the activated carbon-based filter 25 and the housing cap 49. In this manner, the gasket prevents airflow from short-circuiting the activated carbon-based filter 25. Said another way, the gasket causes air to flow through the activated carbon-based filter 25.

The hybrid filter 30 disclosed herein can be configured to substantially mimic the appearance, size and weight of long-used NATO-styler 40 mm canister material-based filters or other similar styles of material-based filters, such as those employed by Warsaw Pact forces, Chinese or other forces or civilian entities.

Some embodiments of the hybrid filter 30 employ an ozone scrubber 92 on the airflow exit 40 coupling with the mask port 12. Accordingly, higher levels of ozone can be generated within the electro-ionic filter 10 to achieve self-cleaning, self-sanitizing, and activated carbon reactivation by simple internal voltage modulation, which in turn can generate significant internal levels of oxidative degradation for toxic agents retained within the hybrid filter 30, including the various elements (e.g., activated carbon-based filter 25, particulate filter 90, ozone scrubber 92) of the hybrid filter 30 and the components of the electro-ionic filter 10. Such capabilities can improve the logistics of the replacement and/or disposal of the material-based filter elements that otherwise would be replete with retained bio-toxic chemicals or virulent bio-pathogens, mandating their disposal instead of reuse and presenting serious hazards to the disposal teams charged with the handling and destruction of such hazardous waste.

In summary, in some embodiments of the hybrid filter 30, the activated carbon-based filter 25 acts to retain and adsorb airborne toxic chemicals, and the ozone and hydroxyl ions generated as by-products of the particle capture ionization mechanism of the electro-ionic filter 10 will in turn oxidize and degrade such toxins retained by the activated carbon-based filter 25.

Various metallic particles and their oxides can act as catalysts and accelerate toxin degradation and oxidation reactions. As noted above, the ozone scrubber 92 may be used to degrade ozone and prevent ozone from entering and irritating the respiratory tract. In other embodiments, such an ozone scrubber 92 may employ cobalt oxide (CoO) instead of manganese oxide. In yet another embodiment, the ozone scrubber 92 may employ both cobalt oxide and manganese oxide.

In some embodiments, the activated carbon-based filter 25 may itself be enhanced to facilitate entrapped toxin degradation by admixing with the activated carbon a number of metallic granules which in turn will improve the toxin degradation by their catalytic accelerator proclivity. The concentrations of such metals (Cu, Mn, Co, Pl, Ag, Pa, etc) and their respective oxides can be customized for a desired acceleration of chemical degradation especially when targeting a specific family of toxic substances in gaseous form.

While the ionization action of the electro-ionic filter 10 is effective in capturing various aerosols, it is possible that toxins may remain in aerosol form to impact the activated carbon-based filter 25. The toxins in gaseous form or vapor will be retained within the activated carbon layer and the metallic catalysts will accelerate the oxidative degradation of such toxins in both their captured aerosol form as well as their gaseous form.

Generally speaking, the hybrid filter 30 can be considered a form of catalytic converter that uses ionization instead of heat as the energy input, as compared to an automotive catalytic converter. The use of any one or more of the above-listed rare metals acts as a catalytic accelerator for the hybrid filter 30.

Oxidation catalysis may be conducted by heterogeneous catalysis and/or homogeneous catalysis. In heterogeneous processes, a gaseous substrate and oxygen (or air) are passed over solid catalysts. Typical catalysts are platinum, and redox-active oxides of iron, vanadium, and molybdenum. In many cases, catalysts are modified with a host of additives or promoters that enhance rates or selectivities.

Homogeneous catalysts for the oxidation of organic compounds may include carboxylates of cobalt, iron, and manganese. To confer good solubility in the organic solvent, these catalysts are often derived from naphthenic acids and ethylhexanoic acid, which are highly lipophilic. These catalysts initiate radical chain reactions, autoxidation that produce organic radicals that combine with oxygen to give hydroperoxide intermediates. Generally, the selectivity of oxidation is determined by bond energies. For example, benzylic C—H bonds are replaced by oxygen faster than aromatic C—H bonds. These concepts associated with homogeneous catalysis and heterogeneous catalysis may be employed in the activated carbon-based filter 25 and/or the ozone scrubber 92 as described above.

As can be understood from FIGS. 2-4, 9 and 10, in use inhaled air enters the intake grille 34 and passes into the multi-layered and spaced-apart stack of electrode grids 70, 74, 78, 82, 86 confined in the housing 50 of the hybrid filter 30 and forming the operative components of the electro-ionic filter 10. Ionization forces generated by the electrode grids 70, 74, 78, 82, 86 of the electro-ionic filter 10 result in electrostatic precipitation that causes airborne particles to deposit on the walls of the cylindrical spacer sections 88A, 88B, 88C, 88D and the P electrode grids 70, 78, 86.

In some circumstances, the air may contain toxic gases that are not in the form of a mist. Such chemicals will be caught within the upper layer of the activated carbon-based filter 25. In some embodiments, this charcoal layer may be smaller in volume than current 40 mm canisters because the carbon in the embodiments disclosed herein are for capture of the toxic molecules and then use the internally modulated ozone generation of the electro-ionic filter 10 to oxidize and degrade the retained toxic molecules irrespective of whether they were retained on the P grid surface or adhered to the charcoal particles, such toxic molecules literarily being bathed in an ozone bath with each breath of the user.

The modulation of ozone can be performed either by increasing the electrode grid voltage of the electro-ionic filter 10 between breaths as measured by an internal airflow sensor or thermocouple. Such an airflow sensor need not be implemented in many situations if the embodiment incorporates a unidirectional airflow Mask with an exhaust valve.

Most gas masks are unidirectional with an exhaust valve to lower the respiratory work because most filters offer increased airflow resistance. In such a situation, the ionizer chamber (which can be considered the interior of the housing 50 containing the electro-ionic filter's electrode grids 70, 74, 78, 82, 86 and the elements (e.g., activated carbon-based filter 25, particulate filter 90, ozone scrubber 92), and where toxic particles are sequestered) will accumulate elevated ozone levels when no air is flowing through the hybrid filter 30 because the user is exhaling through the exhaust port of the unidirectional mask.

After such momentary accumulation of ozone, upon resumption of airflow through the hybrid filter 30 on account of the user inhaling, a bolus of elevated ozone travels through the activated carbon that has retained the toxic gas molecules and actively oxidizes the toxic molecules and in turn reactivates a good portion of the charcoal to capture toxic gas passing through it with inhalation flow. The elevated bolus of ozone within the device has now cleansed the charcoal and encounters degradation via the ozone scrubber 92 (e.g., manganese oxide (MnO₂) filter) or via MnO₂ admixtured within the activated carbon-based filter 25 or particulate filter 90. What now enters the respiratory mask 20 is air that has been cleaned of airborne particles, aerosols and toxic gases.

The coupling of the active ionizer technology disclosed herein with existing material-based protective technology offers a greatly expanded protective performance both in its protective spectrum and its prolonged functionality in a hostile environment, as compared to current mask canisters having protective capacity that is short lived by virtue of saturation of the charcoal bonding sites. Further, the ionizer technology disclosed herein allows the charcoal capture capacity to be refreshed in near real time.

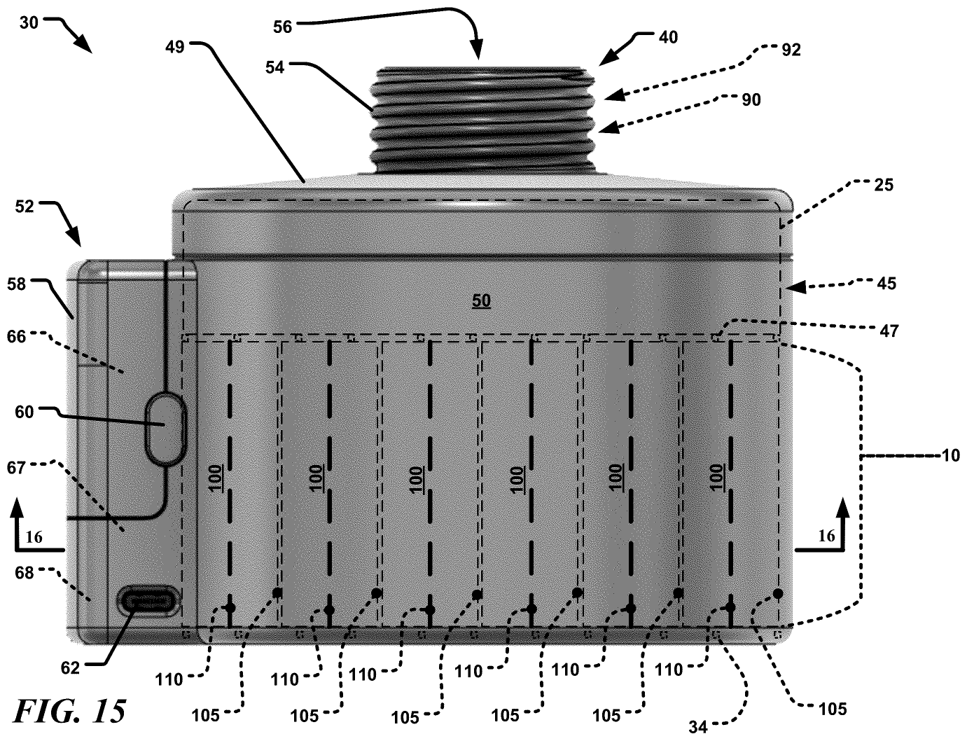

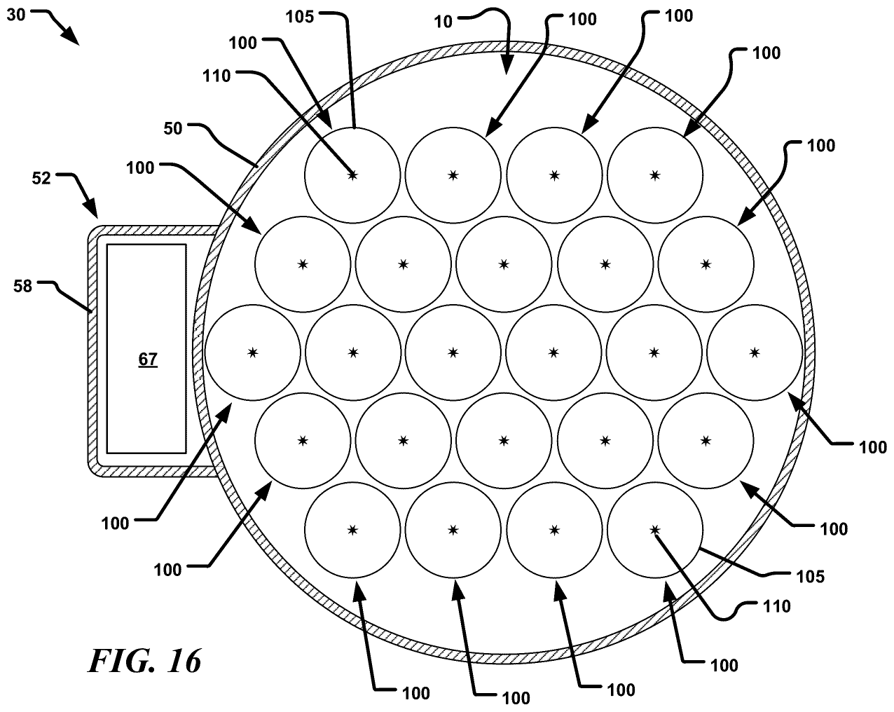

Many of the above-described embodiments pertain to electro-ionic filters 10 employed in series with elements (e.g., activated carbon-based filter 25, particulate filter 90, ozone scrubber 92) to form a hybrid filter 30 where the electro-ionic filters 10 have electrodes 70, 74, 78, 82, 86 stacked in series as described above with respect to FIGS. 9 and 12. However, hybrid filters 30 can also employ other electro-ionic filter arrangements. An example of such an alternative electro-ionic filter arrangement is shown in FIGS. 15 and 16, which are respectively a side elevation of such an alternative electro-ionic filter arrangement and a cross-section as taken along section line 16-16 in FIG. 15.

As can be understood from FIGS. 15 and 16, the electro-ionic filter 10 employs a plurality of cylindrical electrode modules 100 arranged in parallel relative to each other between the intake grille 34 and the inner grille 47. Each cylindrical electrode module 100 includes an inner negative emitter electrode 110 surrounded by an outer cylindrically shaped positive collector electrode 105. The negative emitter electrode 110 may be in the form of a wire or other elongated linear configuration. The inner negative emitter electrode 110 is centered within the outer cylindrically shaped positive collector electrode 105 to be evenly spaced from the inner cylindrical surface of the positive electrode 105 in all radial directions. The above recited materials for the electrodes, the offset spacings between positive and negative electrodes, and the operational voltages may be applied to the embodiment depicted in FIGS. 15 and 16. Also, the teachings in PCT/US2022/071174 (titled Electro-Ionic Mask Devices For Improved Protection From Airborne Biopathogens; filed Mar. 15, 2022) and PCT/US2022/071169 (titled Electro-Ionic Systems And Methods For Treating Enclosed Spaces And Medical Air And Gas Supply Devices For Improved Protection From Airborne Biopathogens; filed Mar. 15, 2022) as they can be applied to the embodiment depicted in FIGS. 15 and 16 are incorporated by reference herein in their entireties.

In other embodiments, the positive collector electrode(s) and negative emitter electrode(s) may be in an arrangement like a spiral staircase, with the electrodes spiraling in a space-apart condition where an offset spacing between the adjacent electrodes is constant along the spiral length of the spiral arrangement. Other configurations are envisioned that create a uniform electric field where distance between the negative emitter electrode and the positive collector electrode are maintained throughout the assembly.

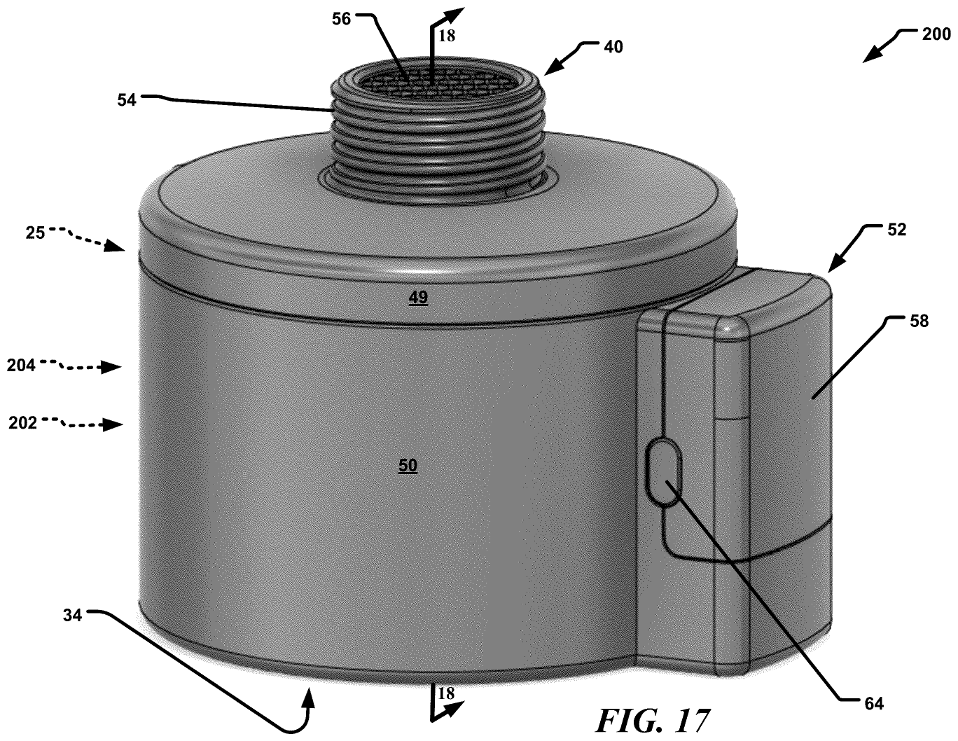

Turning now to FIGS. 17-20, a filter 200 that includes an ozone generator 202 (e.g., ozone generator 202, ozone generator 202′, ozone generator 202″) and a humidity source 204 is illustrated. The filter 200 (as illustrated for example in FIGS. 17-20) can be used in a same or similar manner as the hybrid filter 30 (as illustrated for example in FIGS. 1-16 and as previously described). For example, the filter 200 (as illustrated in FIGS. 17-20) can be operatively coupled to the first port 12 of a mask 20 (as illustrated in FIG. 1) as part of the overall filtration system 15.

It should be noted that the ozone generator 202 can be configured such that its primary purpose is to generate ozone. That is, the ozone generator 202 is not configured to provide filtration. In some aspects, the operational voltage of the ozone generator 202 is between approximately 2 kV and 100,000 kV. In some aspects, the operational voltage of the ozone generator 202 is between approximately 2 kV and approximately 60,000 kV. On the other hand, the electro-ionic filter 10 (as previously described) can be configured such that its primary purpose is to provide electro-ionic filtration. That is, the generation of ozone by the electro-ionic filter 10 is a byproduct of (or otherwise incidental to) the electro-ionic filtration. In some aspects, the operational voltage of the electro-ionic filter 10 is between approximately 2 kV and approximately 12 kV. Thus, in some aspects, the operational voltage of the ozone generator is greater than the operational voltage of the electro-ionic filter 10.

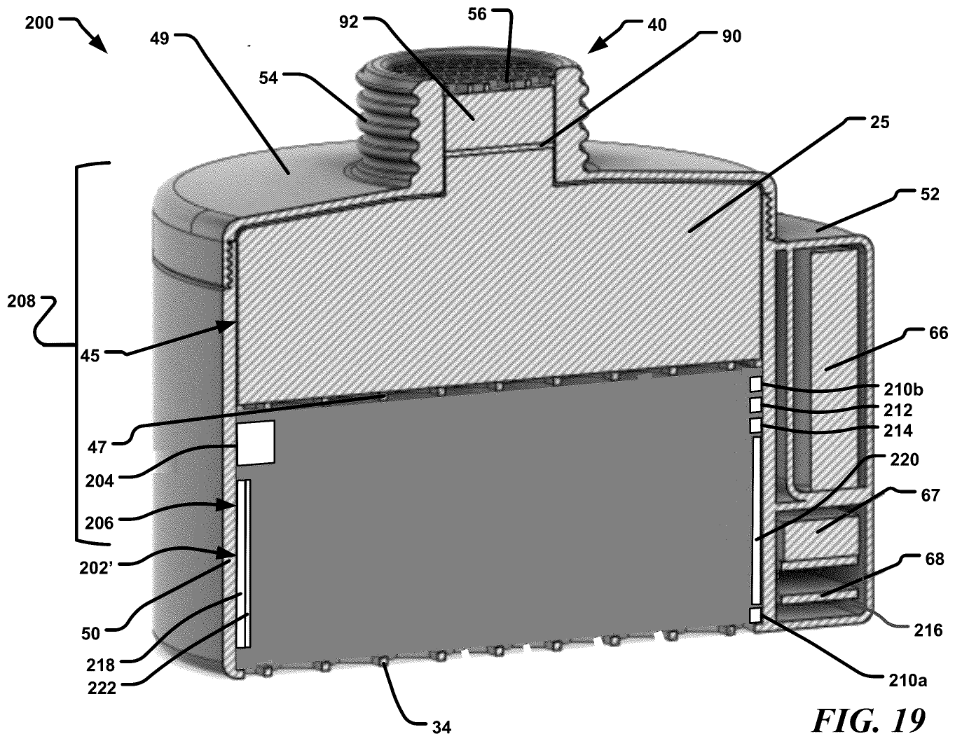

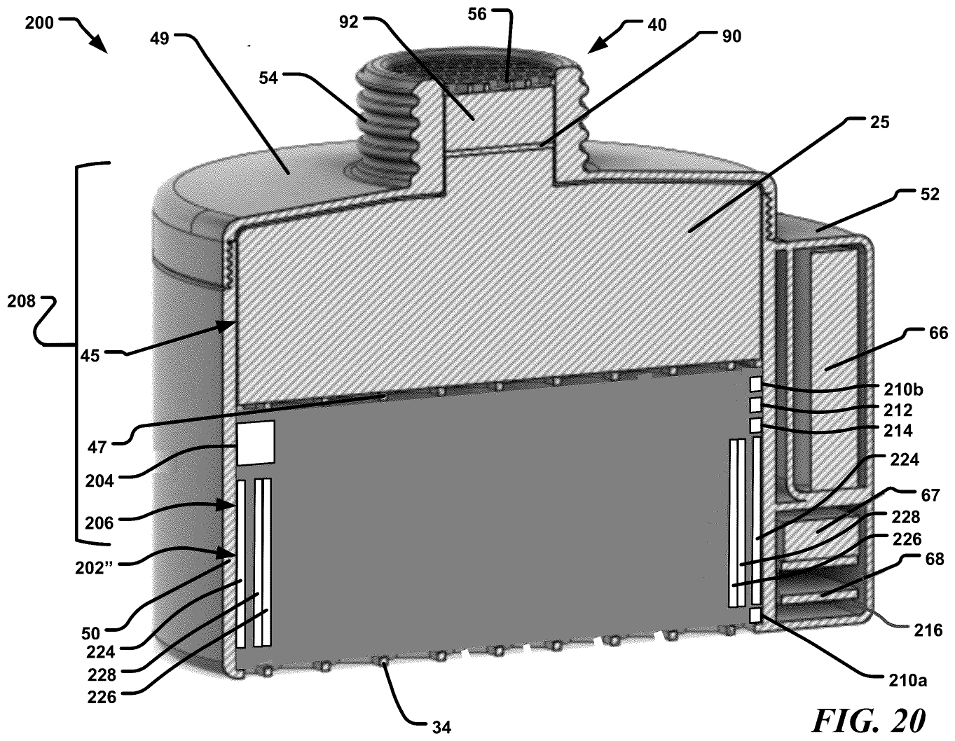

The filter 200 (as illustrated for example in FIGS. 17-20) can include one or more same or similar features, components, or elements as the hybrid filter 30 (as illustrated for example in FIGS. 1-16 and as previously described). For example, the filter 200 can include one or more activated carbon-based filter 25 (e.g., activated carbon filter), one or more particulate filters 90 (e.g., N95 filter) and/or an ozone scrubber 92 (e.g., manganese oxide material-based filter), each of which are described above. FIGS. 17 and 18 show, respectively, a top-side perspective view of such a filter 200 and a cross section as taken along section lines 18-18 in FIG. 17. FIG. 19 illustrates the filter 200 with one example of an ozone generator 202′. FIG. 20 illustrates the filter 200 with another example of an ozone generator 202″.

Without departing from the teaching in the present disclosure, the various components of the filter 200 can be combined into a different arrangement of components, and the filter 200 can include more or less components without limitation. Similarly, the various components of the hybrid filter 30 (as previously discussed with respect to FIGS. 1-16) can be combined into a different arrangement of components, and the hybrid filter 30 can include more or less components without limitation.

In some embodiments, the filter 200 (as illustrated in FIGS. 17-20) can include an electro-ionic filter 10 (as illustrated in FIGS. 1-16) in addition to the ozone generator 202. Similarly, the hybrid filter 30 (as illustrated in FIGS. 1-16) can include the ozone generator 202 (as illustrated in FIGS. 17-20). In these embodiments, the ozone generator 202 can produce ozone in addition to the ozone that is incidentally generated by the ionization functionality of the electro-ionic filter. In some embodiments, the hybrid filter 30 or filter 200 includes a humidity source 204 (as illustrated for example in FIGS. 17-20).

In other embodiments, as illustrated in FIGS. 17-20, the filter 200 includes an ozone generator 202 and does not include an electro-ionic filter. In these embodiments, the ozone generator 202 generates ozone without any ozone incidentally generated since the filter 200 does not include an electro-ionic filter. In some of these embodiments, the filter 200 includes a humidity source 204.

As illustrated in FIGS. 17-20, the filter 200 can include an ozone generator 202 (e.g., ozone generator 202, ozone generator 202′, ozone generator 202″), one or more activated carbon-based filter 25 (e.g., activated carbon filter), one or more particulate filter 90 (e.g., N95 filter), and an ozone scrubber 92 (e.g., manganese oxide material-based filter). In some embodiments, the filter 200 can also include a humidity source 204 (e.g., humidifier).

In short, the ozone generator 202 introduces ozone into the airflow, which flows through the filter 200. Then, each element (activated carbon-based filter 25, particulate filter 90) is exposed to the ozonated airflow, which sanitizes the activated carbon-based filter 25 and/or particulate filter 90. Finally, the ozone scrubber 92 scrubs ozone from the airflow, such that the mask wearer does not inhale an irritating amount of ozone. That is, the volume within the housing 50 of the filter 200, and between the ozone generator 202 and the ozone scrubber 92, defines an ozonated chamber 208 (which contains an increased level of ozone when the ozone generator 202 is generating ozone). The humidity source 204, when present, introduces humidity into the airflow such that the humidity enters the ozonated chamber 208.

Turning to the ozone generator 202 (e.g., ozone generator 202, ozone generator 202′, ozone generator 202″), while continuing with FIGS. 17-20, the filter 200 can include an ozone generator 202 configured to produce (or otherwise generate) ozone. When the ozone generator 202 produces ozone, the ozone is introduced (or otherwise delivered) into the airflow (e.g., airflow 32 illustrated in FIGS. 2-4 and FIG. 14).

In some embodiments, the ozone generator includes an ultra-violet (UV) emitter configured to generate ozone. In some aspects, the ozone generator 202 generates UV radiation. In one example, a solid-state UV emitter (similar to an LED) is configured to emit UV. When UV strikes a surface in the presence of air, ozone is generated as well as other oxidative ions (e.g., OH-) when striking a surface coated with titanium oxide in the presence of humidity. In some embodiments, the solid-state UV emitter generates a relatively low level of ozone and related activated oxidative ions. In some embodiments, the filter 200 includes a UV emitter and does not include a high voltage emitter/collector.

In some embodiments, the ozone generator 202 includes one or more electrodes configured to generate ozone. As non-limiting examples, the electrodes can include one or more pointed elongated members, one or more wires, one or more mesh grids, one or more plates, or the like. These components are arranged to generate a high voltage electric field. In some aspects, the ozone generator 202 includes an anode and a cathode.

In some embodiments, the operational principles of the ozone generator 202 involve high voltage and electron emission such that a corona discharge is not typically observed; nevertheless, it can be considered a micro corona discharge. The current can be controlled with a computing device 216 (e.g., controller, microcontroller) such that corona discharge is seldom observed. The operation produces efficient electron emission, but rarely any spark.

An example of an ozone generator 202′ is illustrated in FIG. 19, according to embodiments of the present disclosure. The ozone generator 202′ includes a first electrode 218 (e.g., high voltage electrode) and a second electrode 220 (e.g., ground electrode) opposite the first electrode 218. A dielectric 222 (e.g., glass, ceramic) is coupled to the first electrode 218. Although FIG. 19 illustrates the cross-section, each of the first electrode 218, the second electrode 220, and the dielectric 222 are plates that are substantially parallel to each other. The ozone generator 202′ can generate a micro corona discharge, which generates ozone. During the micro corona discharge, an electrical discharge occurs between the first electrode 218 and the second electrode 220. During the electrical discharge, the dielectric 222 distributes the electron flow over a greater area across the air gap.

Another example of an ozone generator 202″ is illustrated in FIG. 20, according to embodiments of the present disclosure. The ozone generator 202″ illustrated in FIG. 20 includes an outer electrode 224 and an inner electrode 226 disposed within the outer electrode 224. A dielectric 228 (e.g., glass, ceramic) is coupled to the inner electrode 226. Although FIG. 20 illustrates the cross section, each of the outer electrode 224, the inner electrode 226, and the dielectric 228 are cylindrical and coaxial. The ozone generator 202″ can generate a micro corona discharge, which generates ozone. During micro corona discharge, an electrical discharge occurs between the outer electrode 224 and the inner electrode 226. During the electrical discharge, the dielectric 228 distributes the electron flow over a greater area across the air gap.

The ozone (e.g., produced by the ozone generator 202) is introduced into the airflow at a location upstream from each of the activated carbon-based filter 25 and/or particulate filter 90. In this manner, when airflow is drawn through the filter 200 (e.g., through the intake grille 34, through the chamber 206, through the chamber 45, and through the airflow exit 40), the activated carbon-based filter 25 and/or particulate filter 90 are exposed to the ozone. The ozone sanitizes the bio-pathogens and oxidizes the biohazardous mist retained by the activated carbon-based filter 25 (e.g., activated carbon filter) and/or particulate filter 90 (e.g., N95 filter).

In some embodiments, the ozone generator 202 is disposed within a chamber 206, which is revealed in FIGS. 18-20 due to the removal of the electro-ionic filter. The chamber 206 is bounded on the sides by the inner cylindrical circumferential surface of the housing 50. In some aspects, the chamber 206 is further defined by the intake grille 34 and/or the inner grille 47. For example, the chamber 206 can be defined between the downstream side of the intake grille 34 and/or the upstream side of the inner grille 47. The chamber 206 (which can include the ozone generator 202 disposed therein) is in fluid communication with the chamber 45 (which can include the activated carbon-based filter 25 and/or particulate filter 90 disposed therein). In some aspects, such as when the inner grille 47 is removed, the chamber 206 and the chamber 45 are one continuous chamber.

The ozone generator 202 is fluidly coupled to (or otherwise in fluid communication with) the chamber 206 such that, when the ozone generator 202 generates ozone, the ozone can be delivered into the chamber 206 (and introduced into the airflow flowing therethrough). In some embodiments, the ozone generator 202 is coupled to the housing 50. In some examples, the ozone generator 202 is coupled to the interior of the housing 50 (e.g., within the chamber 206), as illustrated for example in FIGS. 18-20. In other examples, the ozone generator 202 is coupled to the exterior of the housing 50 (i.e., apart from the chamber 206). In some embodiments, the ozone generator 202 is coupled to the electronics box 52 (e.g., coupled to the interior of the electronics box 52, coupled to the exterior of the electronics box 52). When the ozone generator 202 is apart from the chamber 206 (e.g., outside the housing 50), the housing 50 can include an opening (or otherwise pathway) therethrough to establish fluid communication between the ozone generator 202 and the chamber 206.

In some examples, the ozone generator 202 includes a fan configured to direct (e.g., push via positive fan pressure, pull via negative fan pressure) the ozone through the filter 200 (e.g., through the activated carbon-based filter 25 and/or particulate filter 90). In other examples, the ozone is drawn through the filter 200 when the wearer of the mask breathes in (i.e., inhales).

Turning to the one or more filtration elements (e.g., activated carbon-based filter 25, particulate filter 90), while continuing with FIGS. 17-20, the filter 200 can include one or more filtration elements. For example, the filter can include a activated carbon-based filter 25 (e.g., activated carbon material-based filter) and/or a particulate filter 90 (e.g., N95 style material-based filter) disposed therein. Each filtration element is configured to retain and adsorb airborne toxic molecules, toxic chemicals, and the like from the airflow.

Each filtration element (e.g., activated carbon-based filter 25, particulate filter 90) can include one or more same or similar features as discussed previously with respect to FIGS. 1-16. For example, the filtration elements can retain contaminants of the airflow by entrapping the contaminants therein. Then, ozone (e.g., generated by the ozone generator 202) that is drawn through the filtration element neutralizes the contaminants trapped therein. When the filtration elements include an activated carbon-based filter 25, the activated carbon-based filter 25 can be reactivated when it is exposed to the ozone. In this manner, the ozone generator 202 can prolong the useful service life of the filtration elements. If the ozone generator 202 ceases operation (e.g., fails), the filtration elements can continue to function with essentially a full use life remaining when the ozone generator 202 ceased functioning.

The filtration elements (e.g., activated carbon-based filter 25, particulate filter 90) are located downstream relative to the ozone generator 202 and upstream from the ozone scrubber 92. In this manner, the filtration elements are exposed to the ozone as the airflow is drawn through the filter 200. The ozone produced by the ozone generator 202 can have same or similar effects on the filter elements as the ozone produced by the electro-ionic filter (as previously discussed with respect to FIGS. 1-16). For example, the ozone can regenerate carbon by oxidative degradation of toxic molecules retained by the activated carbon-based filter 25.

Said another way, the filtration elements are disposed within the ozonated chamber 208 (e.g., within the housing 50 and between the ozone generator 202 and the ozone scrubber 92). In some aspects, the ozonated chamber 208 includes at least a portion of chamber 206 and/or at least a portion of chamber 45. Because the ozonated chamber 208 contains an increased level of ozone when the ozone generator 202 is generating ozone, the filtration elements are exposed to the ozone.

In some examples, one or more of the filtration elements (e.g., activated carbon-based filter 25, particulate filter 90) includes water and/or a pH buffering agent. When included in the filtration element, the water adds humidity (e.g., water vapor), which can enhance the function of ozone, as discussed below. In some examples, the water is added to (e.g., pre-wetted, impregnated into) the activated carbon-based filter 25 (e.g., a water impregnated activated carbon material-based filter). When included in at least one of the filtration elements, the pH buffering agent helps neutralize toxic gas agents, such as ammonia (NH4) or chlorine (Cl2), among others. In some examples, the pH buffering agent includes sodium bicarbonate (also referred to as NaHCO₃, baking soda, etc.). In some examples, the pH buffering agent is added to (e.g., impregnated into) the activated carbon-based filter 25. In at least one example, the activated carbon-based filter 25 (e.g., charcoal filter) includes both water and a pH buffering agent. In some aspects, the water and the pH buffering agent are added to the activated charcoal. The baking soda can help neutralize toxic gas agents and/or toxins that form from the chemical process of toxins passing into the charcoal and/or ozone scrubber 92.

In some examples, the filtration elements (e.g., activated carbon-based filter 25, particulate filter 90) are disposed within the chamber 45. In some aspects, the chamber 45 is further defined by the inner grille 47 and/or the housing cap 49. For example, the chamber 45 can be defined between the downstream side of the inner grille 47 and/or the housing cap 49. In some aspects, the activated carbon-based filter 25 is located within a lower portion of the chamber 45 and the particulate filter 90 is located within an upper portion of the chamber 45.

Turning now to the ozone scrubber 92, while continuing with FIGS. 17-20, the filter 200 can include an ozone scrubber 92. The ozone scrubber 92 is configured to scrub (or otherwise degrade) ozone (e.g., produced by the ozone generator 202) such that the ozone is reduced (if not eliminated) from the airflow. In some embodiments, the ozone scrubber 92 includes manganese oxide (e.g., a manganese oxide filter). In some aspects, manganese oxide serves as a catalytic degrader (scrubber) of ozone that is generated by the ozone generator 202. For example, the ozone scrubber 92 can include a grid that is impregnated with manganese oxide. In some embodiments, the ozone scrubber 92 includes cobalt oxide (e.g., a cobalt oxide filter).

The ozone scrubber 92 is located downstream relative to each of the one or more filtration elements (e.g., activated carbon-based filter 25, particulate filter 90). In this manner, the airflow on the upstream side of the ozone scrubber 92 includes ozone (e.g., produced by the ozone generator 202) such that the filtration elements are exposed to the ozone. That is, as the airflow is drawn through the filter 200, the ozone passes through the filtration elements.

Because the ozone scrubber 92 scrubs the ozone from the airflow, the airflow on the downstream side of the ozone scrubber 92 includes a reduced level of ozone (or, in some cases, no ozone) when compared to the airflow on the upstream side of the ozone scrubber 92. In this manner, after passing through the ozone scrubber 92, the airflow can include a concentration of ozone that is not irritating to the respiratory tract. In some examples, the concentration is 0.2 parts per million (ppm) or less. In some examples, the concentration is 0.1 ppm or less.

In some aspects, the ozone scrubber 92 is located within the upper portion of the chamber 45. In some aspects, the ozone scrubber 92 is located upstream relative to the exit grille 56, which extends across the opening of the airflow exit 40. In this manner, the ozone scrubber 92 scrubs ozone from the airflow before the airflow exits the airflow exit 40.

Turning now to the humidity source 204, while continuing with FIGS. 17-20, the filter 200 can include a humidity source 204. The humidity source 204 is configured to increase the concentration of water vapor in the airflow (e.g., add humidity). In some aspects, higher humidity fosters greater ozone oxidative activity. In some aspects, the humidity source 204 is configured to introduce humidity into the ozonated chamber 208 (within the housing 50 and between the ozone generator 202 and the ozone scrubber 92), which contains an increased level of ozone when the ozone generator 202 is generating ozone. Humidity (e.g., water vapor) can enhance the functionality of ozone, in some cases approximately 4-times (4x) that of ozone that is not humidified (e.g., in the absence of humidity). Thus, the humidity source 204 can enhance the functionality of the ozone such that the ozone is more effective in sanitizing the filtration elements (e.g., activated carbon-based filter 25, particulate filter 90). In this manner, the humidity source 204 can help prolong the useful service life of the filtration elements.

Similar to the hybrid filter 30 (as previously discussed), the filter 200 can be setup for unidirectional airflow or bidirectional airflow depending on the specific mask setup. It should be noted that the breath of the mask wearer incidentally produces humidity, as discussed below.

In some embodiments, such as when the mask is set up for bidirectional airflow, the ozonated chamber 208 (e.g., chamber 206, chamber 45) receives humidity when the mask wearer breathes out (i.e., exhales). That is, exhaled air flows through the filter 200 in the opposite, or otherwise reverse, direction of the airflow 32 (as illustrated for example in FIG. 3 and FIG. 14 and described with respect to when the wearer breathes in (i.e., inhales)). In some examples, the humidity source 204 generates humidity that is in addition to (or otherwise in conjunction with) the humidity that is incidentally generated by the breath of the wearer. In other examples, the filter 200 relies on humidity from the breath of the wearer alone (i.e., the filter 200 does not include a supplemental humidity source 204).

In some embodiments (e.g., dry climate operations), at least some exhaled air is diverted back through the filter 200. When the mask is set up for unidirectional airflow through the filter 200, exhaled air can be diverted back through the filter by slightly increasing exhalation valve opening pressure of the mask. In some examples, a thin coating of a material (e.g., hydrogel) is applied to the surface of the exhalation valve which increases the stickiness, thereby increasing the opening pressure, of the exhalation valve. In this manner, at least some of the relatively higher humidity air, which was exhaled by the wearer, passes through the filter 200. As noted above, the exhaled air can flow along the airflow 32, but in the opposite direction of the inhaled air path as described for the airflow 32 (as illustrated for example in FIG. 3 and FIG. 14).

In some embodiments, such as when the mask is setup for unidirectional airflow (as illustrated with example mask 20 having an exhaust valve in FIGS. 1-2), the ozonated chamber 208 does not receive humidity from the breath of the mask wearer because the wearer exhales through the exhaust port (e.g., exhaust valve) of the unidirectional mask. In these embodiments, the humidity source 204 can generate humidity alone (i.e., without humidity incidentally produced by the breath of the wearer).

The humidity source 204 is in fluid communication with the ozonated chamber 208 (which can include at least a portion of chamber 206 and/or at least a portion of chamber 45) such that the humidity can be delivered into the ozonated chamber 208 (and introduced into the airflow flowing therethrough). That is, the humidity can be introduced downstream from the ozone generator 202 and upstream from the ozone scrubber 92. In this manner, the humidity interacts with the ozone, which is produced by the ozone generator 202 and reduced (or eliminated) by the ozone scrubber 92. That is, when the humidity source 204 introduces humidity to the airflow and the ozone generator 202 introduces ozone to the airflow, each of the filtration elements (e.g., activated carbon-based filter 25, particulate filter 90) are exposed to airflow that is both ozonated and humidified. That is, the airflow includes both an increased concentration of ozone (from the ozone generator 202) and an increased concentration of humidity (from the humidity source 204). The humidity can enhance the functionality of the ozone in sanitizing the filtration elements. In some aspects, the humidity increases the efficiency of the filter 200 such that the battery performance (e.g., battery life) is enhanced.

In some embodiments, the humidity source 204 is apart from (or separated from) the filter 200. For example, the humidity source 204 can be coupled to the mask, the wearer of the mask, etc., directly or indirectly, and can include a tube or conduit that fluidly couples the humidity source 204 to the filter 200 (e.g., ozonated chamber 208), thereby establishing fluid communication therebetween. In one example, the humidity source 204 is strapped to the wearer and a tube connects the humidity source 204 to the ozonated chamber 208 of the filter 200.

In some embodiments, the humidity source 204 is coupled to the housing 50. In some examples, the humidity source 204 is coupled to the interior of the housing 50, such as within the ozonated chamber 208 (e.g., within chamber 206, within chamber 45), as illustrated for example in FIGS. 18-20. In other examples, the humidity source 204 is coupled to the exterior of the housing 50 (i.e., apart from chamber 206, apart from chamber 45). In some embodiments, the humidity source 204 is coupled to the electronics box 52 (e.g., coupled to the interior of the electronics box 52, coupled to the exterior of the electronics box 52). When the humidity source 204 is apart from the ozonated chamber 208 (e.g., outside the housing 50), the housing 50 can include an opening (or otherwise pathway) therethrough to establish fluid communication between the humidity source 204 and the ozonated chamber 208.

In some embodiments, the humidity source 204 includes a reservoir (or otherwise tank) configured to retain water (e.g., via holding). In some aspects, the reservoir can include a port (e.g., an opening having a removable cap) such that the reservoir can be refilled with water when the water supply is depleted. In some embodiments, the humidity source includes a sponge configured to retain water (e.g., via absorption). In some aspects, the humidity source 204 is a humidity generator, such as, for example, a humidifier. For example, the humidifier can include an evaporative humidifier, an ultrasonic humidifier, and/or a warm mist humidifier. In some examples, the humidity source 204 includes a filter. In some examples, the humidity source 204 includes a fan configured to propel water vapor (e.g., evaporated water). In some examples, the humidity source 204 includes a heating element configured to raise the temperature (e.g., boil) of the retained water.

In some embodiments, the humidity source 204 is a water-impregnated filtration element (e.g., water impregnated activated carbon-based filter 25, water-impregnated particulate filter 90). For example, the activated carbon-based filter 25 can include carbon (e.g., charcoal) that is impregnated with water.

Turning now to the sensors, while continuing with FIGS. 18-20, the filter 200 can include one or more sensors (e.g., toxin sensors 210, ozone sensors 212, humidity sensors 214). Each sensor can monitor one or more corresponding parameters (e.g., toxin parameters, ozone parameters, humidity parameters) of the airflow (e.g., airflow 32 illustrated in FIGS. 2-4 and FIG. 14). It should be noted that, although the sensors are illustrated as included in filter 200 (as illustrated for example in FIG. 18-20), the same or similar sensors can be included in hybrid filter 30 (as illustrated for example in FIGS. 1-16).

One or more toxin sensors 210 (e.g., toxin sensor 210a, toxin sensor 210b), as illustrated for example in FIGS. 18-20, can be included in the filter 200. Each toxin sensor 210 is configured to monitor one or more toxin parameters (e.g., type of toxin, concentration of toxin) within the airflow. And each toxin sensor 210 is in communication with the computing device 216 (e.g., controller, microcontroller), such that each toxin sensor 210 can provide toxin parameters to the computing device 216. In some embodiments, the filter 200 includes one toxin sensor 210. In other embodiments, the filter 200 includes two or more toxin sensors 210.

In some embodiments, as illustrated in FIGS. 18-20, at least one toxin sensor 210 is disposed between the intake grille 34 and the exit grille 56 (e.g., coupled to the interior of the housing 50), such that it can monitor toxin parameters of the airflow within the filter 200. In some aspects, a toxin sensor 210 (e.g., toxin sensor 210a) is disposed upstream of the ozone generator 202 (or upstream of the electro-ionic filter 10 as illustrated in FIGS. 1-16), such that it is outside the ozonated chamber 208. Additionally or separately, a toxin sensor 210 (e.g., toxin sensor 210b) is disposed downstream of the ozone generator 202 (or downstream of the electro-ionic filter 10 as illustrated in FIGS. 1-16), such that it is within the ozonated chamber 208.

The position of the toxin sensors 210 as illustrated in FIGS. 18-20 should not be construed as limiting. In some embodiments, a toxin sensor 210 is disposed within chamber 45, which contains activated carbon-based filter 25. In some examples, a toxin sensor 210 is embedded in activated carbon-based filter 25. In some examples, a toxin sensor 210 is embedded in particulate filter 90. In some embodiments, at least one toxin sensor 210 is disposed upstream from the intake grille 34, such that it can monitor toxin parameters of the airflow before entering the filter 200. In some embodiments, at least one toxin sensor 210 is disposed downstream from the exit grille 56, such that it can monitor toxin parameters of the airflow after exiting the filter 200.

One or more ozone sensors 212, as illustrated for example in FIGS. 18-20, can be included in the filter 200. Each ozone sensor 212 is configured to monitor one or more ozone parameters (e.g., concentration of ozone) within the airflow. And each ozone sensor 212 is in communication with the computing device 216 (e.g., controller, microcontroller), such that each ozone sensor 212 can provide ozone parameters to the computing device 216. In some embodiments, the filter 200 includes one ozone sensor 212. In other embodiments, the filter 200 includes two or more ozone sensors 212.

In some embodiments, as illustrated in FIGS. 18-20, at least one ozone sensor 212 is disposed between the intake grille 34 and the exit grille 56 (e.g., coupled to the interior of the housing 50), such that it can monitor ozone parameters of the airflow within the filter 200. In some aspects, an ozone sensor 212 is disposed downstream of the ozone generator 202 (or downstream of the electro-ionic filter 10 as illustrated in FIGS. 1-16), such that it is within the ozonated chamber 208.

The position of the ozone sensor 212 as illustrated in FIGS. 18-20 should not be construed as limiting. In some embodiments, an ozone sensor 212 is disposed within chamber 45, which contains activated carbon-based filter 25. In some examples, an ozone sensor 212 is embedded in activated carbon-based filter 25. In some examples, an ozone sensor 212 is embedded in particulate filter 90. In some embodiments, at least one ozone sensor 212 is disposed downstream from the exit grille 56, such that it can monitor ozone parameters of the airflow after exiting the filter 200.

One or more one or more humidity sensors 214, as illustrated for example in FIGS. 18-20, can be included in the filter 200. Each humidity sensor 214 is configured to monitor one or more humidity parameters (e.g., relative humidity) within the airflow. And each humidity sensor 214 is in communication with the computing device 216 (e.g., controller, microcontroller), such that each humidity sensor 214 can provide humidity parameters to the computing device 216. In some embodiments, the filter 200 includes one humidity sensor 214. In other embodiments, the filter 200 includes two or more humidity sensors 214.

In some embodiments, as illustrated in FIGS. 18-20, at least one humidity sensor 214 is disposed between the intake grille 34 and the exit grille 56 (e.g., coupled to the interior of the housing 50), such that it can monitor humidity parameters of the airflow within the filter 200. In some aspects, a humidity sensor 214 (is disposed downstream of the ozone generator 202 (or downstream of the electro-ionic filter 10 as illustrated in FIGS. 1-16), such that it is within the ozonated chamber 208.

The position of the humidity sensor 214 as illustrated in FIGS. 18-20 should not be construed as limiting. In some embodiments, a humidity sensor 214 is disposed within chamber 45, which contains activated carbon-based filter 25. In some examples, a humidity sensor 214 is embedded in activated carbon-based filter 25. In some examples, a humidity sensor 214 is embedded in particulate filter 90. In some embodiments, at least one humidity sensor 214 is disposed downstream from the exit grille 56, such that it can monitor ozone parameters of the airflow after exiting the filter 200.

One or more temperature sensors (not shown), such as a thermocouples, can be included in the filter 200. The temperature sensor can be configured to detect different portions of the respiratory cycle (e.g., inhalation, exhalation) of the wearer of the mask. And each temperature sensor is in communication with the computing device 216 (e.g., controller, microcontroller), such that each temperature sensor can provide temperature parameters (which can be correlated to the respirator cycle) to the computing device 216.

In some embodiments, the electronics can be modulated in accordance with the respiratory cycle. For example, the electronics can cause the production of ozone to be decreased during inhalation and increased during exhalation. In some examples, the ozone production can be increased after inhalation is complete, thereby increasing the amount of ozone within the filter 200. Increasing the amount of ozone in this manner can increase the dwell efficacy of the ozone.

In some embodiments, such as when the mask has its own exhalation vent, there is a natural dwell time of ozone within the filter 200 between inspirations. That is, exhaled airflow does not travel in reverse through the filter 200, which allows the ozone to dwell within the filter 200 during exhalation. In some of these embodiments, because of the natural dwell time of the ozone, the filter 200 does not include a temperature sensor to monitor the respiratory cycle of the wearer.

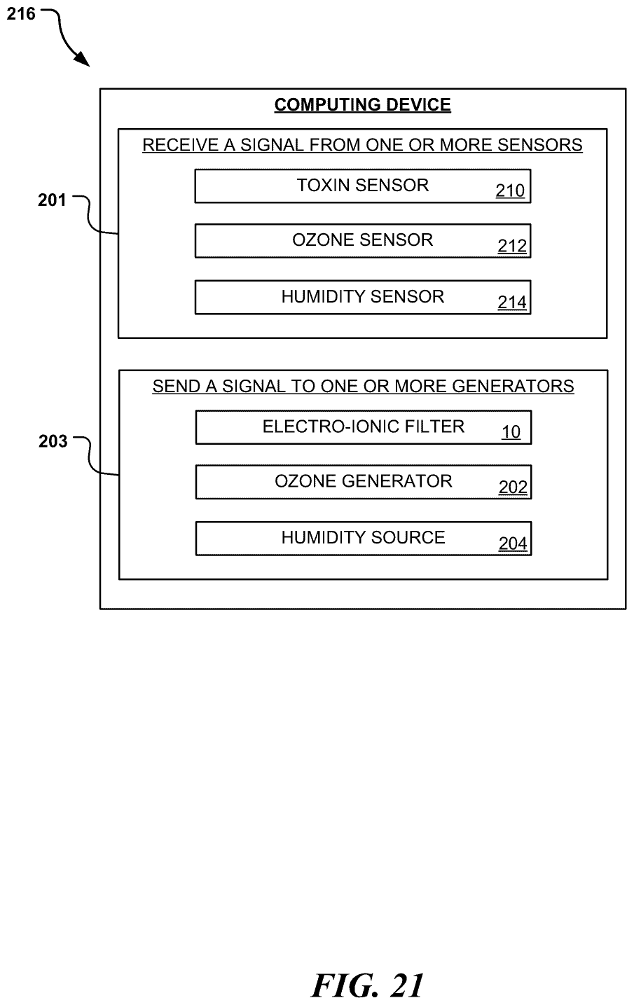

Turning now to the computing device 216, while continuing with FIGS. 18-20 and also discussing FIG. 21, the filter 200 can include a computing device 216 (e.g., controller, microcontroller). In some embodiments, as illustrated for example in FIGS. 18-20, the computing device can include one or more PCBs 67, 68 containing the circuitry, memory, and processor that runs the filter 200. In some embodiments, as illustrated for example in FIGS. 18-20, the computing device 216 is disposed within the electronics box 52 of the filter 200.

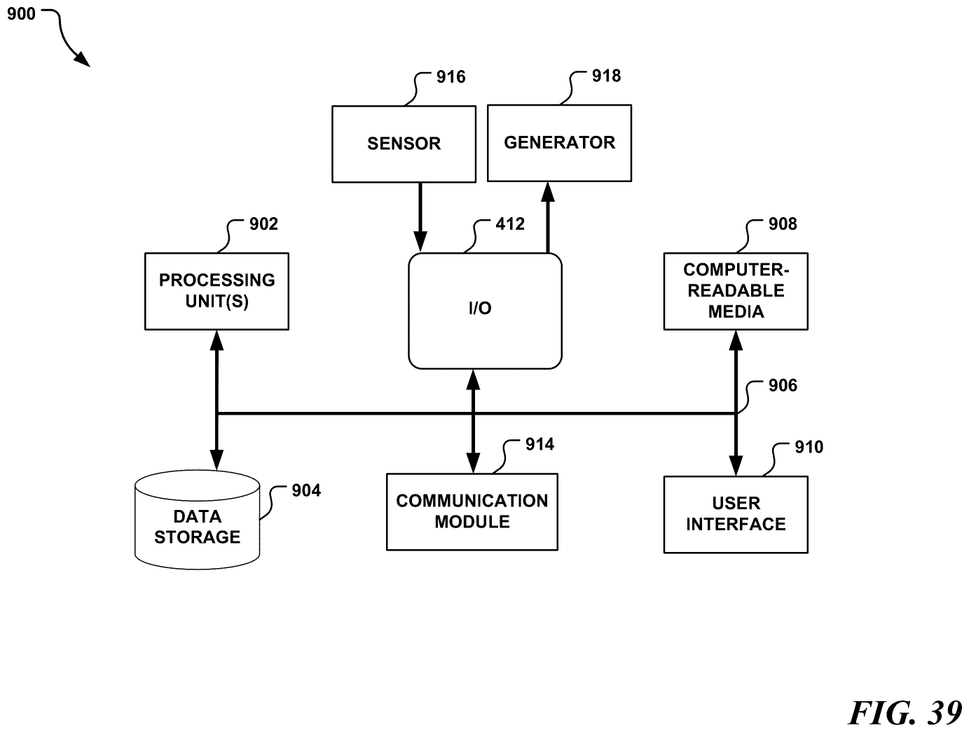

In some aspects, the computing device 216 (as illustrated for example in FIGS. 18-21) can include one or more same or similar components as the computing device 900 (as illustrated for example in FIG. 39). It should be noted that, although the computing device 216 is illustrated as included in filter 200 (as illustrated for example in FIGS. 18-20), the same or similar computing device 216 can be included in hybrid filter 30 (as illustrated for example in FIGS. 1-16).

The computing device 216 (e.g., input of the computing device 216) is in communication with each of the one or more sensors (e.g., toxin sensors 210, ozone sensors 212, humidity sensors 214). In this manner, as illustrated in block 201 in FIG. 21, the computing device 216 can receive a signal from each sensor. Each signal can include the corresponding parameters (e.g., toxin parameters, ozone parameters, humidity parameters) of the airflow (e.g., airflow 32 illustrated in FIGS. 2-4 and FIG. 14) monitored by the respective sensor.

The computing device 216 (e.g., output of the computing device 216) is in communication with each of the generators (e.g., electro-ionic filter 10, ozone generator 202, humidity source 204). In this manner, as illustrated in block 203 in FIG. 21, the computing device 216 can send a signal to each generator. Each signal can adjust (or otherwise modulate) the generator. That is, in response to one or more parameters received from a toxin sensor 210, an ozone sensor 212, and/or a humidity sensor, the computing device 216 can modulate the electro-ionic filter 10 (e.g., modulate voltage), the ozone generator 202 (e.g., modulate voltage), and/or the humidity sensor 214 (e.g., modulate fan speed).

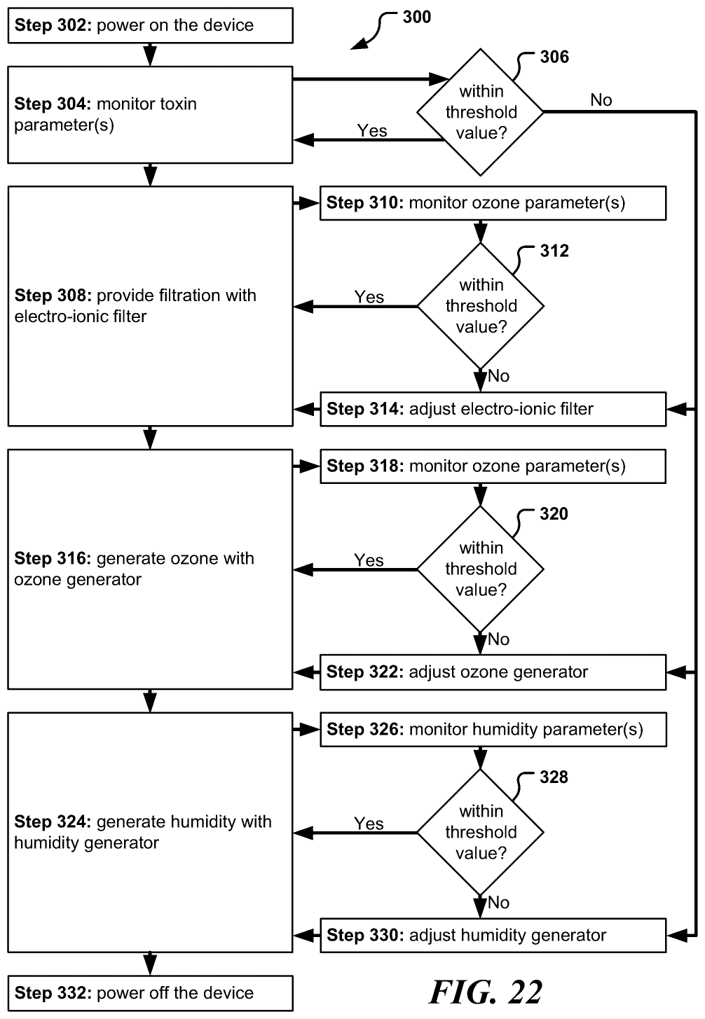

Turning now to FIG. 22, an example method 300 is illustrated in a flowchart, according to one embodiment. Each block shown in FIG. 22 can represent one or more processes, methods, or subroutines, carried out in the example method 300. Furthermore, the illustrated order of blocks is illustrative only and the order of the blocks can change according to the present disclosure. Additional blocks can be added or fewer blocks can be utilized, without departing from this disclosure.

In some embodiments, the method 300 (also referred to as the control sequence) is used in conjunction with the hybrid filter 30 (illustrated for example in FIGS. 1-16). For example, the method 300 can control the electro-ionic filter 10 of the hybrid filter 30. In some embodiments, the method 300 is used in conjunction with the filter 200 (illustrated for example in FIGS. 17-20). For example, the method 300 can control the ozone generator 202 of the filter 200.

At block 302 (continuing with FIG. 22), the device (e.g., hybrid filter 30, filter 200) is powered on. In some embodiments, the user (e.g., the wearer of mask 20, as illustrated in FIG. 1) can power the device on. In some examples, the power button 60 is actuated (e.g., depressed) to power on the device. In some examples, before or after powering on the device, power can be connected to the power cord receptacle 62 to charge (or recharge) the rechargeable battery (or batteries) within the device. In some examples, the access lid 58 can be removed and the battery (or batteries) within the electronics box 52 can be removed and replaced.

When the device is powered on, the status indicator 64 can indicate the status of the device. The status indicator 64 can include one or more indicators (e.g., power indicator, charge indicator, ozone indicator, filter indicator). In turn, each indicator indicates one or more corresponding statuses (e.g., power status, charge status, ozone generation status, filter status) of the device. In some examples, a power indicator indicates the power status (e.g., powered on, powered off) of the device. In some examples, a charge indicator (e.g., charge level/percentage) indicates the charge status of the device. In some examples, an ozone generation indicator (e.g., generating ozone, not generating ozone) indicates the ozone generating status of the device.

Turning now to block 304, as illustrated in FIG. 22, one or more toxin parameters (e.g., type of toxin, concentration of toxin) can be monitored. In some embodiments, one or more toxin sensors 210 (as illustrated for example in FIGS. 18-20) can monitor the toxin parameters. The toxin parameters pertain to toxin with the airflow into the filter, through the filter, and/or out of the filter.

At block 306, a determination can be made whether each toxin parameter is within a threshold value (e.g., above a lower threshold value, below an upper threshold value). In some embodiments, the computing device 216 (as illustrated for example in FIGS. 18-21) receives the toxin parameters from the toxin sensors 210. If the toxin parameters are within the threshold value, toxin monitoring can continue at block 304 as previously discussed. If one or more toxin parameters are not within a threshold value, the electro-ionic filter can be adjusted (at block 314, for example, as discussed below), the ozone generator can be adjusted (at block 322, for example, as discussed below), and/or the humidity generator can be adjusted (at block 330, for example, as discussed below).

Turning now to block 308, as illustrated in FIG. 22, electro-ionic filtration can be provided. In some embodiments, the electro-ionic filter 10 (as illustrated for example in FIGS. 1-16 and as previously discussed) provides electro-ionic filtration. That is, the electro-ionic filter 10 can filter contaminants (which are in the airflow) out of the airflow. In this manner, the filtered-out contaminants do not reach the filtration elements (e.g., activated carbon-based filter 25, particulate filter 90). In some examples, the electro-ionic filter 10 can generate ozone, which can neutralize contaminants trapped within the filtration elements.

At block 310, one or more ozone parameters can one or more ozone parameters (e.g., concentration of ozone) can be monitored. In some embodiments, one or more ozone sensors 212 (as illustrated for example in FIGS. 18-20) can monitor the ozone parameters. The ozone parameters pertain to ozone within the airflow into the filter, through the filter, and/or out of the filter.

At block 312, a determination can be made whether each ozone parameter is within a threshold value (e.g., above a lower threshold value, below an upper threshold value). In some embodiments, the computing device 216 (as illustrated for example in FIGS. 18-21) receives the ozone parameters from the ozone sensors 212. In some examples, the lower threshold value is approximately 0.005 ppm of ozone. In some examples, the upper threshold value is approximately 0.2 ppm of ozone. In some examples, the upper threshold value is approximately 0.1 ppm of ozone. If the ozone parameters are within the threshold value, electro-ionic filtration can continue at block 308 as previously discussed.

In some aspects, the filter is configured such that the concentration of ozone inhaled by the wearer is less than 0.1 parts per million (ppm). For example, the filter can be configured such that the concentration of ozone passing through the ozone scrubber 92 (e.g., manganese oxide filter) is less than 0.1 ppm. Because the ozone scrubber 92 is configured to scrub or otherwise reduce ozone from the airflow, the concentration of ozone in the ozonated chamber can be greater than 0.1 ppm. The relatively higher levels of ozone within the ozonated chamber can kill retained retain bio pathogens and degrade toxic chemicals. In some aspects, the relatively higher levels of ozone can refresh activated charcoal.

At block 314, if one or more ozone parameters are not within a threshold value, the electro-ionic filter can be adjusted. In some embodiments, the computing device 216 (as illustrated for example in FIGS. 18-21) adjusts the electro-ionic filter 10 (e.g., adjusts the voltage). For example, if an ozone parameter was determined to be below the lower threshold value at block 312, the electro-ionic filtration can be increased (e.g., increase the voltage). If an ozone parameter was determined to be above the upper threshold value at block 312, the electro-ionic filtration can be decreased (e.g., decrease the voltage). In this manner, the electro-ionic filtration can be adjusted in response to one or more ozone parameters.

Continuing with block 314, if one or more of the toxin parameters are not within a threshold value (as determined at block 306), the electro-ionic filter can be adjusted. For example, if the toxin parameters were determined to be above the upper threshold value at block 306, the electro-ionic filtration can be increased. In this manner, the electro-ionic filtration can be adjusted in response to one or more toxin parameters. After the adjustment at block 314, electro-ionic filtration can continue at block 308 as previously discussed. In some cases, the electro-ionic filtration can be shut off at block 314.

Turning now to block 316, as illustrated in FIG. 22, ozone can be generated. In some embodiments, the ozone generator 202 (as illustrated for example in FIGS. 18-20 and as previously discussed) generates ozone. The ozone is introduced into the airflow (e.g., within the filter) and can neutralize contaminants trapped within the filtration elements (e.g., activated carbon-based filter 25, particulate filter 90).

At block 318, one or more ozone parameters (e.g., concentration of ozone) can be monitored. In some embodiments, one or more ozone sensors 212 (as illustrated for example in FIGS. 18-20) can monitor the ozone parameters. The ozone parameters pertain to ozone within the airflow into the filter, through the filter, and/or out of the filter.

At block 320, a determination can be made whether each ozone parameter is within a threshold value (e.g., above a lower threshold value, below an upper threshold value). In some embodiments, the computing device 216 (as illustrated for example in FIGS. 18-21) receives the ozone parameters from the ozone sensors 212 In some examples, the lower threshold value is approximately 0.005 ppm of ozone. In some examples, the upper threshold value is approximately 0.2 ppm of ozone. In some examples, the upper threshold value is approximately 0.1 ppm of ozone. If the ozone parameters are within the threshold value, ozone generation can continue at block 316 as previously discussed.

At block 322, if one or more ozone parameters are not within a threshold value, the ozone generator can be adjusted. In some embodiments, the computing device 216 (as illustrated for example in FIGS. 18-21) adjusts the ozone generator 202 (e.g., adjusts the voltage). For example, if an ozone parameter was determined to be below the lower threshold value at block 320, the ozone generation can be increased (e.g., increase the voltage). If an ozone parameter was determined to be above the upper threshold value at block 320, the ozone generation can be decreased (e.g., decrease the voltage). In this manner, the generation of ozone can be adjusted in response to one or more ozone parameters.

Continuing with block 322, if one or more of the toxin parameters are not within a threshold value (as determined at block 306), the ozone generator can be adjusted. For example, if the toxin parameters were determined to be above the upper threshold value at block 306, the ozone generation can be increased. In this manner, the generation of ozone can be adjusted in response to one or more toxin parameters. After the adjustment at block 320, ozone generation can continue at block 316 as previously discussed. In some cases, the ozone generation can be shut off at block 322.

Turning now to block 324, as illustrated in FIG. 22, humidity can be generated. In some embodiments, the humidity source 204 (as illustrated for example in FIGS. 18-20 and as previously discussed) generates humidity. The humidity is introduced into the airflow (e.g., within the filter) and can enhance the functionality of ozone in the airflow.

At block 326, one or more humidity parameters (e.g., relative humidity) can be monitored. In some embodiments, one or more humidity sensors 214 (as illustrated for example in FIGS. 18-20) can monitor the humidity parameters. The humidity parameters pertain to humidity within the airflow into the filter, through the filter, and/or out of the filter.

At block 328, a determination can be made whether each humidity parameter is within a threshold value (e.g., above a lower threshold value, below an upper threshold value). In some embodiments, the computing device 216 (as illustrated for example in FIGS. 18-21) receives the humidity parameters from the humidity sensors 214. If the humidity parameters are within the threshold value, humidity generation can continue at block 324 as previously discussed.

At block 330, if one or more humidity parameters are not within a threshold value, the humidity generator can be adjusted. In some embodiments, the computing device 216 (as illustrated for example in FIGS. 18-21) adjusts the humidity source 204 (e.g., adjusts the speed of the fan). For example, if a humidity parameter was determined to be below the lower threshold value at block 328, the humidity generation can be increased (e.g., increase the fan speed). If a humidity parameter was determined to be above the upper threshold value at block 328, the humidity generation can be decreased (e.g., decrease the fan speed). In this manner, the generation of humidity can be adjusted in response to one or more humidity parameters.

Continuing with block 330, if one or more of the toxin parameters are not within a threshold value (as determined at block 306), the humidity generator can be adjusted. For example, if the toxin parameters were determined to be above the upper threshold value at block 306, the humidity generation can be increased. In this manner, the generation of humidity can be adjusted in response to one or more toxin parameters. After the adjustment at block 330, humidity generation can continue at block 324 as previously discussed. In some cases, the humidity generation can be shut off at block 330.

At block 332 (continuing with FIG. 22), the device (e.g., hybrid filter 30, filter 200) can be powered off. In some embodiments, the user (e.g., the wearer of mask 20, as illustrated in FIG. 1) can power the device off. In some examples, the power button 60 is actuated (e.g., depressed) to power off the device. In some examples, before or after powering off the device, power can be connected to the power cord receptacle 62 to charge (or recharge) the rechargeable battery (or batteries) within the device. In some examples, the access lid 58 can be removed and the battery (or batteries) within the electronics box 52 can be removed and replaced.

In some embodiments, the device can initiate a shutdown mode, which includes a shutdown sequence before the device is powered off. That is, the power (e.g., battery) for the device (e.g., hybrid filter 30, filter 200) remains active and ozone can continue to be generated to recharge the filters for a predetermined time. The generation of ozone when the device is not being used provides an additional concentrated ozone bath of any residual chemical(s) captured in the carbon particles.

In some examples, the device can continue to run for a predetermined period of time (e.g., between approximately 5 minutes and approximately 10 minutes) after the device enters the shutdown mode (e.g., after the user actuates the power button 60) and before the device actually powers off. In some examples, the devices can continue to run for a period of time determined by the device (e.g., computer) based on one or more variables (e.g., total run time, ozone generation levels, etc.). For example, the computer can determine an amount of time for the device to continue to generate ozone to regenerate the filters. In this manner, the shutdown sequence can reactivate (or otherwise regenerate the absorptive capacity of) the activated charcoal filter and/or KN95 filter that contain chemical toxins and/or pathogens.

In some embodiments, similar to the shutdown mode previously described, the device can initiate a self-cleaning mode when the device is charging (e.g., plugged in to an electrical power supply). During the self-cleaning mode, the device can generate ozone for a predetermined period of time (e.g., between approximately 5 minutes and approximately 10 minutes). In this manner, the self-cleaning sequence can reactivate (or otherwise regenerate the absorptive capacity of) the activated charcoal filter.

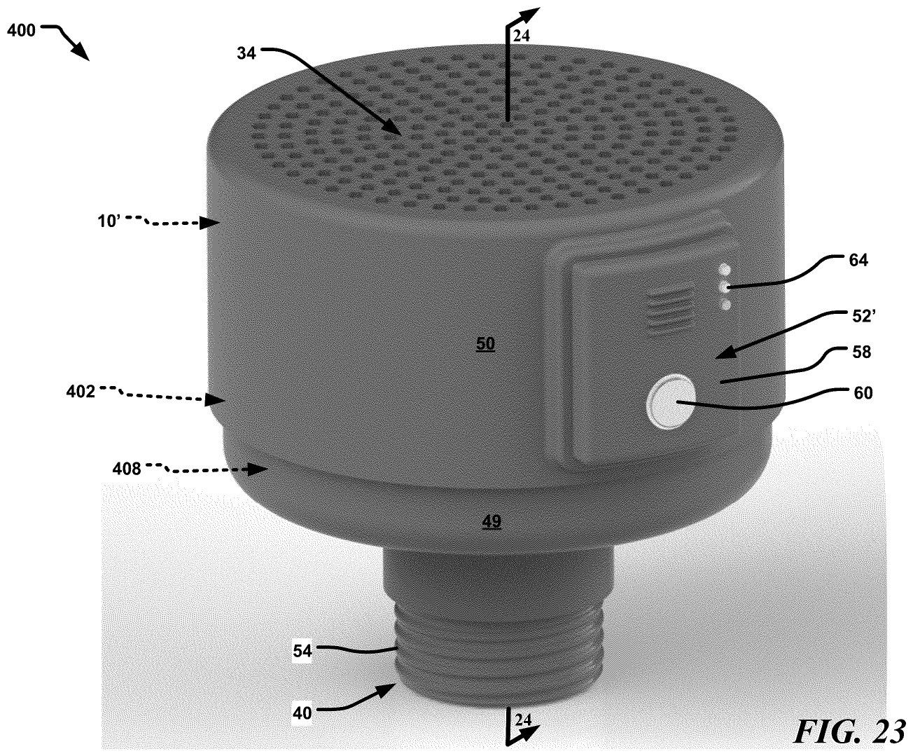

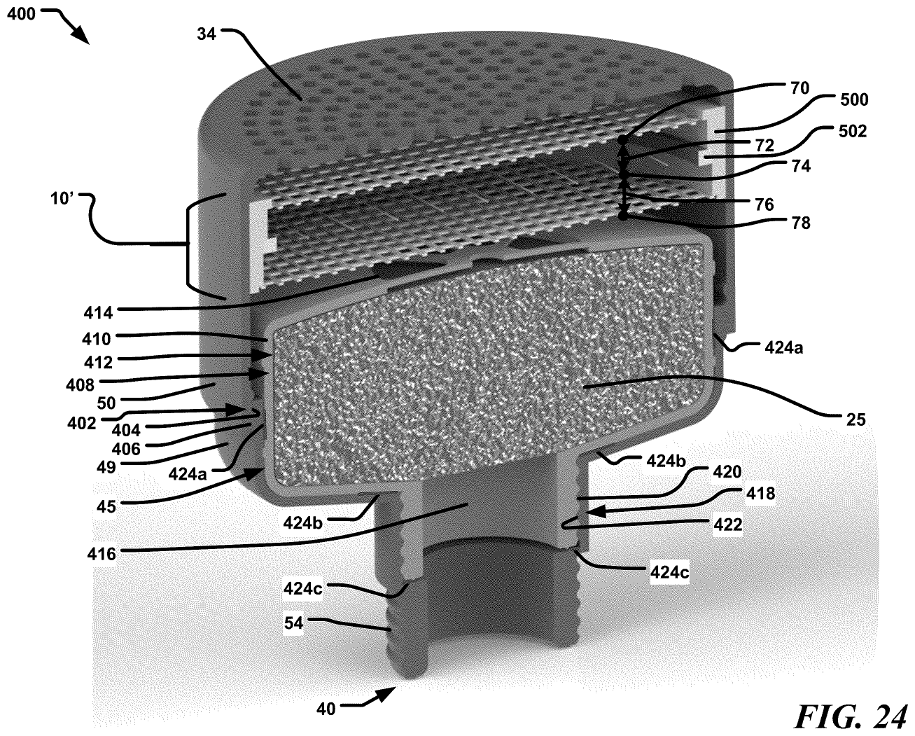

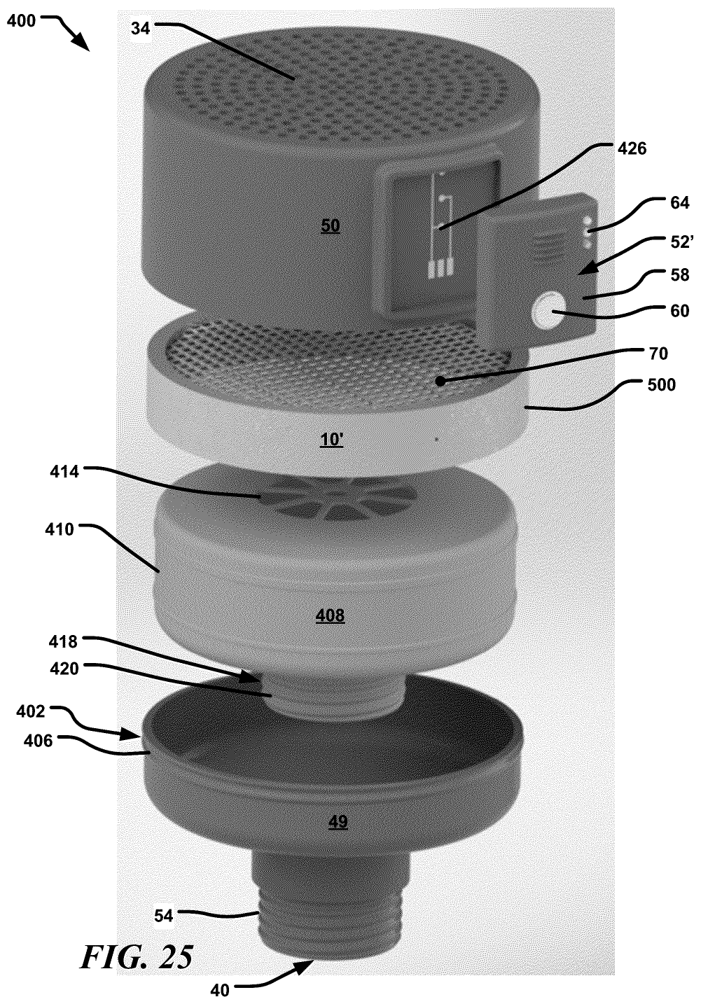

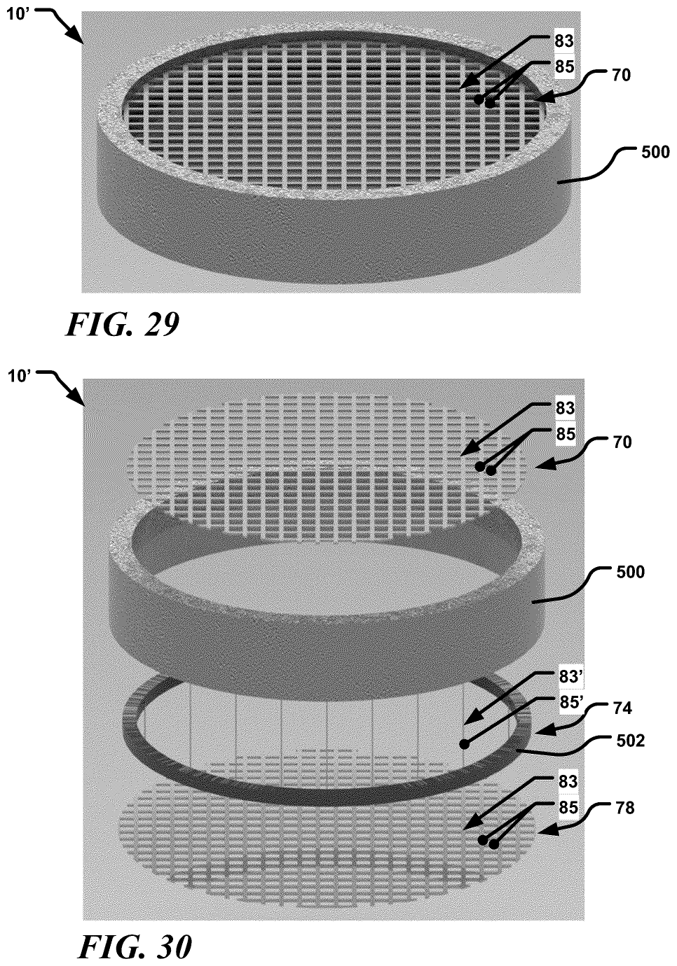

Turning now to FIGS. 23-25, a hybrid filter 400 is illustrated. The hybrid filter 400 includes an electro-ionic filter 10′ and a filter insert 408 (which includes a activated carbon-based filter 25) oriented in series therein. The hybrid filter 400 is shown in a bottom-side perspective view (in FIG. 23), in a cross-sectional perspective view as taken along section line 24-24 in FIG. 23 (in FIG. 24), and in an exploded view (in FIG. 25). Without departing from the teaching in the present disclosure, the various components of the hybrid filter 400 can be combined into a different arrangement of components, and the hybrid filter 400 can include more or less components without limitation. The electro-ionic filter 10′ is further discussed below with respect to FIGS. 29-30.

The hybrid filter 400 (as illustrated for example in FIGS. 23-25) can be used in a same or similar manner as the hybrid filter 30 (as illustrated for example in FIGS. 1-16 and as previously described) and/or the filter 200 (as illustrated for example in FIGS. 17-20 and as previously described). For example, the hybrid filter 400 (as illustrated for example in FIGS. 23-25) can include one or more same or similar features, components, or elements as the hybrid filter 30 (as illustrated for example in FIGS. 1-16 and as previously described) and/or as the filter 200 (as illustrated for example in FIGS. 17-20 and as previously described). Moreover, the hybrid filter 400 can employ a method that is the same as or similar to the method 300 (as illustrated for example in FIG. 22 and as previously discussed).

Continuing with FIGS. 23-25, the hybrid filter 400 includes an attachment mechanism 402 to removably couple the housing cap 49 (also referred to as the lower housing or the first housing portion) to the housing 50 (also referred to as the upper housing or the second housing portion). In this manner, the housing cap 49 and housing 50 can be removably coupled together (e.g., connected such that the hybrid filter 400 can be used), as illustrated for example in FIGS. 23-24. The housing cap 49 and housing 50 can be uncoupled (e.g., disconnected such that the internal components of the hybrid filter 400 are accessible), as illustrated for example in FIG. 25. In this manner, the hybrid filter 400 is modular in that components (e.g., electro-ionic filter 10′, filter insert 408) are interchangeable. In some embodiments, when the housing cap 49 is removably coupled to the housing 50, the airflow exit 40 of the housing cap 49 is coaxial with the airflow inlet (e.g., intake grille 34) of the housing 50.

The attachment mechanism 402 can include threads, latches, bayonet connectors, interference fits, a hinge and latch, or the like. That is, the attachment mechanism 402 is not limited to the threads 404, 406, as illustrated for example in FIG. 24. For example, the housing cap 49 and housing 50 can be clam-shelled together with a hinge (such that the housing cap 49 and housing 50 can open and close with respect to each other) and include a latch opposite the hinge (such that the housing cap 49 and housing 50 can be secured in the closed position).

In some embodiments, a gasket (not shown in the figures) can be included at the joint between the housing cap 49 and housing 50 to impede, or otherwise prevent, air from flowing through the joint. In some examples, the gasket is an o-ring. In this manner, the gasket prevents airflow from short-circuiting the hybrid filter 400. Said another way, the gasket causes air to flow from the intake grille 34 to the airflow exit 40, such that the air flows through the electro-ionic filter 10′ and through the filter insert 408. In some examples, the gasket is constructed of polymer (e.g., rubber). Besides acting as a sealant, the gasket can also impede unintentional unthreading and associated airflow short-circuiting and noise in the field from loose components.

In some embodiments, as illustrated for example in FIG. 24, the attachment mechanism 402 includes threads 404 on the housing 50 and corresponding threads 406 on the housing cap 49. The threads 404, 406 are configured to rotatably mate together (e.g., by turning in a first direction (e.g., clockwise)) to removably couple the housing cap 49 and housing 50. As illustrated in FIG. 24, the threads 404 on the housing 50 face radially inward and the threads 406 on the housing cap 49 face radially outward. The outer diameter of the housing cap 49 corresponds to the inner diameter of the housing 50 such that the housing cap 49 is advanced into the housing 50 when the threads 404, 406 are rotatably mated together. The threads 404, 406 are configured to rotatably un-mate (e.g., by turning in a second direction (e.g., counterclockwise) opposite the first direction).

When the housing cap 49 is uncoupled from the housing 50 via attachment mechanism 402 (e.g., the threads 404, 406 are unmated), as illustrated for example in FIG. 25, one or more components (e.g., electro-ionic filter 10′, filter insert 408) of the hybrid filter 400 can be removed and replaced. For example, an inoperative electro-ionic filter 10′ (e.g., electro-ionic filter 10′ at or near the end of its service life) can be removed and replaced with an operative electro-ionic filter 10′ (e.g., new electro-ionic filter 10′). As another example, an inoperative filter insert 408 (e.g., filter insert 408 at or near the end of its service life) can be removed and replaced with an operative filter insert 408 (e.g., new filter insert 408). That is, the inoperative filter insert 408 can be uncoupled from the housing cap 49 via attachment mechanism 418 (e.g., the threads 420, 422 are unmated) and, subsequently, the operative filter insert 408 can be removably coupled to the housing cap 49 via attachment mechanism 418 (e.g., the threads 420, 422 are rotatably mated together).

Continuing with FIGS. 23-25, the hybrid filter 400 can include a filter insert 408 (also referred to as a cartridge or canister). The filter insert 408 includes a housing 410 that defines a chamber 412 therein, as illustrated for example in FIG. 24. The housing 410 includes an inlet 414 and an outlet 416. The inlet 414 can receive airflow into the chamber 412 and the outlet 416 can discharge airflow out of the chamber 412. An activated carbon-based filter 25 (e.g., activated carbon filter) is disposed within the chamber 412 of the filter insert 408. In this manner, air flowing through the chamber 412 correspondingly flows through the activated carbon-based filter 25 therein. In some embodiments, the housing 410 of the filter insert 408 is constructed of polymer, such as, among others, a modified polyphenylene ether-polystyrene blend (PPE&PS).

In some embodiments, the filter insert 408 is a CBRN (chemical, biological, radiological, and nuclear) rated filter. In some embodiments, the filter insert 408 is manufactured by Avon Protection or an equivalent. In some aspects, the filter insert 408 is a CBRNCF50 Filter by Avon Protection. In some aspects, the filter insert 408 is a FM61EU CBRN Filter by Avon Protection. In some aspects, the filter insert 408 is a GPCF50 CBRN Filter by Avon Protection. In some aspects, the filter insert 408 is a CFP100 Particulate Filter by Avon Protection. In some aspects, the filter insert 408 is a CTCF50 Riot Agent Filter by Avon Protection. In some embodiments, the filter insert 408 is an equivalent manufactured by others.

In some embodiments, the shape of the cross-section of the filter insert 408 is generally circular such that the filter insert 408 defines a diameter. In some examples, the diameter of the filter insert 408 is between approximately 100 mm and approximately 130 mm. In some examples, the diameter of the filter insert 408 is between approximately 110 mm and approximately 120 mm. In some aspects, the diameter of the filter insert 408 is approximately 111 mm. In some aspects, the diameter of the filter insert 408 is approximately 113 mm. In some aspects, the diameter of the filter insert 408 is approximately 118 mm.

In some embodiments, the shape of the cross-section of the filter insert 408 is generally rectangular such that the filter insert 408 defines a length and width. In some examples, the length of the filter insert 408 is between approximately 100 mm and approximately 130 mm. In some examples, the length of the filter insert 408 is between approximately 110 mm and approximately 120 mm. In some examples, the length of the filter insert 408 is approximately 117 mm. In some examples, the width of the filter insert 408 is between approximately 80 mm and 110 mm. In some examples, the width of the filter insert 408 is between approximately 90 mm and 100 mm. In some examples, the width of the filter insert 408 is approximately 96 mm.

The hybrid filter 400 includes an attachment mechanism 418 to removably couple the filter insert 408 to the housing cap 49 of the housing 50. In this manner, the filter insert 408 and the housing cap 49 can be removably coupled together (e.g., connected such that the hybrid filter 400 can be used), as illustrated for example in FIGS. 23-24. The filter insert 408 and the housing cap 49 can be uncoupled (e.g., disconnected such that the filter insert 408 can be removed and replaced), as illustrated for example in FIG. 25. In some embodiments, when the filter insert 408 is removably coupled to the housing cap 49, the outlet 416 of the filter insert 408 is coaxial with the airflow exit 40 of the housing cap 49.

The attachment mechanism 418 can include threads, latches, bayonet connectors, interference fits, a hinge and latch, or the like. That is, the attachment mechanism 418 is not limited to the threads 420, 422, as illustrated for example in FIG. 24. Nevertheless, the attachment mechanism can include corresponding threads 420,422, as discussed below.

Continuing with FIGS. 23-25, one or more gaskets 424 (e.g., gasket 424a, gasket 424b, gasket 424c) can be included at the joint between the filter insert 408 (e.g., housing 410 of the filter insert 408) and the chamber 45 (e.g., housing cap 49 of the housing 50), as illustrated for example in the cross-sectional view of FIG. 24. In some examples, the gasket 424 is an o-ring. When present, the gasket 424 impedes, or otherwise prevents, air from flowing through the joint. In this manner, the gasket 424 prevents airflow from short-circuiting the filter insert 408. Said another way, the gasket 424 causes air to flow from the inlet 414, through the filter insert 408 (and the activated carbon-based filter 25 disposed therein), to the outlet 416. In some examples, the gasket 424 is constructed of polymer (e.g., rubber). In some embodiments, the one or more gaskets 424 are integrated into the manufacture of the housing cap 49. In this manner, the removable filter insert 408 can be advanced into (and removably coupled to the housing cap 49) until the filter insert 408 abuts the one or more gaskets 424.

When the filter insert 408 is removably coupled to the housing cap 49, the gasket 424 can inhibit differential movement (e.g., translation, rotation) between the filter insert 408 and the housing cap 49. For example, the gasket 424 can inhibit rattling of the filter insert 408 and/or unthreading of filter insert 408 with respect to the housing cap 49. The position of the gaskets 424 as illustrated in FIG. 24 should not be construed as limiting. Nevertheless, as illustrated for example in FIG. 24, a gasket 424a can be positioned between a sidewall of the chamber 45 (e.g., inner circumferential surface of the housing cap 49) and a corresponding sidewall of the filter insert 408 (e.g., outer circumferential surface of the filter insert 408). Additionally or alternatively, a gasket 424b can be positioned between a base of the chamber 45 (e.g., base of the housing cap 49) and a corresponding base of the filter insert 408. Additionally or alternatively, a gasket 424c can be positioned at the mouth of the filter insert 408 (e.g., between an end of the filter insert 408 and a surface formed by a recess in the housing cap 49).

In some embodiments, as illustrated for example in FIG. 24, the attachment mechanism 418 includes threads 420 on the filter insert 408 and corresponding threads 422 on the housing cap 49 such that the male necked outlet 416 of the housing 410 of the filter insert 408 can be threadably received in the female necked outlet of the housing cap 49 in a mated male/female or nested arrangement. The threads 420, 422 are configured to rotatably mate together (e.g., by turning in a clockwise direction) to removably couple the filter insert 408 and housing cap 49. As illustrated in FIG. 24, the threads 422 on the housing cap 49 face radially inward (i.e., internal threads) and the threads 420 on the filter insert 408 face radially outward (i.e., external threads). In some embodiments, the threads 420, 422 are a 40 mm threaded connection in accordance with NATO STANAG 4155 and/or EN 148-1.

One or more sets of threads 420, 422 can include a thread sealer at the joint between the threads 420, 422. When present, the thread sealer impedes, or otherwise prevents, air from flowing through the joint. In this manner, the thread sealer prevents airflow from short-circuiting the filter insert 408. Said another way, the thread sealer causes air to flow from the inlet 414, through the filter insert 408 (and the activated carbon-based filter 25 disposed therein), to the outlet 416. The thread sealer can be a thread sealant (e.g., anaerobic thread sealer), thread seal tape (e.g., polytetrafluoroethylene (PTFE) tape), or the like. Besides acting as a sealant, the thread sealer can also impede unintentional unthreading and associated airflow short-circuiting and noise in the field from loose components.

In some embodiments, the housing cap 49 includes a neck and a body extending therefrom, as illustrated for example in FIG. 24, which correspond to a neck and body of the housing 410 of the filter insert 408. In this manner, the filter insert 408 can nest within the housing cap 49 when the housing cap 49 receives the filter insert 408 therein.

When the filter insert 408 is removably coupled (e.g., threadably connected) to the housing cap 49, the neck of the filter insert 408 is received by the neck of the housing cap 49. The outer diameter of the neck portion of the filter insert 408 can correspond to the inner diameter of the housing cap 49 such that the filter insert 408 is advanced into the housing cap 49 when the threads 420, 422 are rotatably mated together. In some embodiments, as illustrated for example in FIG. 24, the neck of the housing cap 49 includes a recessed portion, which extends radially outward from the longitudinal axis of the housing cap 49, to receive the neck of the filter insert 408. In some aspects, the thickness of the sidewalls of the neck of the filter insert 408 are substantially the same as the depth (in the radial direction) of the recess. In this manner, the internal diameter defined by the outlet 416 of the filter insert 408 can be substantially the same as the internal diameter defined by the airflow exit 40 of the housing cap 49.

When the filter insert 408 is removably coupled to the housing cap 49, the filter insert 408 is received by the chamber 45. The outer diameter of the housing 410 of the filter insert 408 can correspond to the inner diameter of the housing cap 49 of the housing 50. In this manner, at least a portion of the sidewall of the housing cap 49 (e.g., inner circumferential surface of the housing cap 49) can abut at least a portion of the sidewall of the housing 410 of the filter insert 408 (e.g., outer circumferential surface of the housing 410). The respective sidewalls being in abutting coextensive contact can inhibit differential movement (e.g., translation, rotation) of the filter insert 408 with respect to the housing cap 49. Additionally or alternatively, the base of the housing 410 (which extends radially outward from the neck of the filter insert 408) can correspond to the base of the housing cap 49 (which extends radially outward from the neck of the housing cap 49). In some aspects, when the filter insert 408 is removably coupled to the housing cap 49, at least a portion of the base of the housing 410 (e.g., outer planar surface) abuts at least a portion of the base of the housing cap 49 (e.g., inner planar surface). The respective bases being in abutting coextensive contact can inhibit differential movement (e.g., translation, rotation) of the filter insert 408 with respect to the housing cap 49.

When the hybrid filter 400 is assembled, as illustrated for example in FIGS. 23-24, the filter insert 408 is removably coupled to the housing cap 49. In this manner, the filter insert 408 is disposed within the chamber 45. The housing 50, which can include the electro-ionic filter 10′ therein, is removably coupled to the housing cap 49. When removably coupled together, the housing cap 49 and housing 50 enclose both the electro-ionic filter 10′ and the activated carbon-based filter 25 (e.g., activated carbon filter).

Because the electro-ionic filter 10′ is disposed upstream of the filter insert 408 (and activated carbon-based filter 25 therein), airflow is drawn into the hybrid filter 400 through the intake grille 34 and then the airflow passes through the electro-ionic filter 10′. The electro-ionic filter 10′ can filter contaminants (which are in the airflow) out of the airflow. In this manner, the filtered-out contaminants do not reach the filter insert 408 (and the corresponding activated carbon-based filter 25). In some examples, the electro-ionic filter 10′ can generate ozone, which can neutralize contaminants trapped within the activated carbon-based filter 25. In this manner, the ozone from the electro-ionic filter 10′ can reactivate (or otherwise regenerate the absorptive capacity of) the activated carbon-based filter 25 (e.g., activated charcoal filter).

After the airflow flows through the electro-ionic filter 10′, the airflow enters the filter insert 408 through the inlet 414. Then, the airflow flows through the chamber 412 (and activated carbon-based filter 25 disposed therein) of the filter insert 408. As airflow flows through the activated carbon-based filter 25, the activated carbon-based filter 25 can retain and adsorb airborne toxic molecules, toxic chemicals, and the like from the airflow. Then, airflow flows through the outlet 416 of the filter insert 408 and through the airflow exit 40 of the hybrid filter 400.

In some embodiments, the hybrid filter 400 includes a particulate filter 90 (e.g., N95 filter) and/or an ozone scrubber 92, which are illustrated for example in the hybrid filter 30 illustrated in FIGS. 1-16, located downstream of the filter insert 408. In some embodiments, the hybrid filter 400 includes an exit grille 56 extending across the opening of the airflow exit 40.

Continuing with FIGS. 23-25, the hybrid filter 400 includes an electronics box 52′. The electronics box 52′ (as illustrated for example in FIGS. 23-25) can include one or more same or similar features, components, or elements as the electronics box 52 (as illustrated for example in FIGS. 1-20 and as previously described). The electronics box 52′ can include electronics therein, which can include a computing device 216 (as previously discussed with respect to FIGS. 18-21). The electronics box 52′ can be removably coupled to the housing 50, such that electronics box 52′ engages the connection terminal 426 to establish a connection between the electronics in the electronics box 52′ and the internal electrical components of the hybrid filter 400.

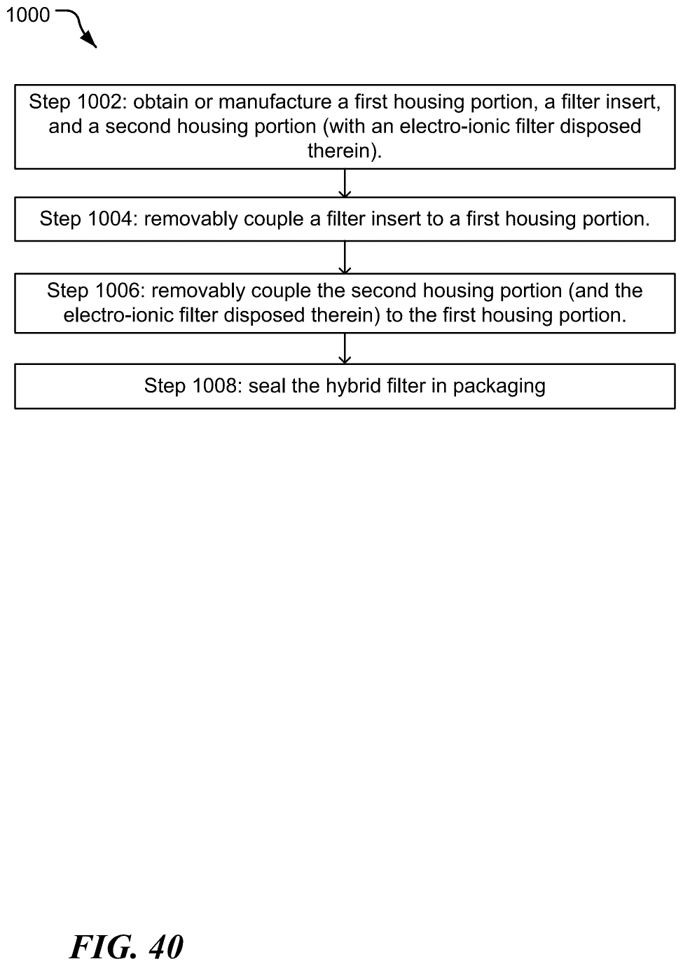

Turning now to FIG. 40, an example method 1000 of factory assembling a hybrid filter 400 is illustrated in a flowchart, according to one embodiment. Each block shown in FIG. 40 can represent one or more processes, methods, or subroutines, carried out in the example method 1000. Furthermore, the illustrated order of blocks is illustrative only and the order of the blocks can change according to the present disclosure. Additional blocks can be added or fewer blocks can be utilized, without departing from this disclosure.

At block 1002, the components of a hybrid filter 400, as illustrated for example in FIGS. 23-25, are obtained or manufactured. The filter insert 408 can be manufactured or obtained from a manufacturer (e.g., first manufacturer). The first housing portion (e.g., housing cap 49) and the second housing portion (e.g., housing 50), which includes the electro-ionic filter 10′ disposed therein, can be manufactured or obtained from a manufacturer (e.g., second manufacturer).

The hybrid filter 400 can be provided in a kit. In some examples, the hybrid filter 400 is provided preassembled within the kit. In a preassembled state, the first housing portion (e.g., housing cap 49), which has a filter insert 408 removably coupled therein, is removably coupled to the second housing portion (e.g., housing 50), which has an electro-ionic filter 10′ disposed therein. To provide a preassembled hybrid filter 400, the factory method can proceed to block 1004. In other examples, the hybrid filter 400 is provided disassembled within the kit (e.g., the first housing portion is not coupled to the second housing portion). To provide a disassembled hybrid filter 400, the factory method can proceed to block 1008.

At block 1004, the filter insert 408 can be removably coupled (e.g., threadably mated) to the first housing portion (e.g., housing cap 49). In some embodiments, one or more gaskets 424 (as illustrated for example in FIG. 24) are positioned at the joint between the filter insert 408 (e.g., housing 410 of the filter insert 408) and the first portion of the housing.

In some embodiments, the second housing portion (e.g., housing 50), which includes an electro-ionic filter 10′ disposed therein, can be uncoupled (e.g., unthreaded) from the first housing portion so that the filter insert 408 can be removably coupled to the first housing portion. Such is the case when the first housing portion and the second housing portion are already coupled together when they are obtained. After uncoupling the second housing portion from the first housing portion, the second housing portion can be separated from the first housing portion to provide access so that the filter insert 408 can be removably coupled within the first housing portion.

At block 1006, the second housing portion (e.g., housing 50), which includes an electro-ionic filter 10′ disposed therein, can be removably coupled (e.g., threadably mated) to the first housing portion (e.g., housing cap 49).

At block 1008, the hybrid filter 400 is sealed within packaging so that the hybrid filter 400 can be provided as a kit. The hybrid filter 400 can be an assembled hybrid filter 400 or a disassembled hybrid filter 400, as previously discussed. In some embodiments, the packaging encloses the hybrid filter 400 and an instruction manual. In some embodiments, more than one filter insert 408 are included in the kit so that the end user has an initial filter insert 408 and at least one replacement filter insert 408. In this manner, the end user can replace the initial filter insert 408 with the replacement filter insert 408 when the initial filter insert 408 is at or near the end of its useful service life.

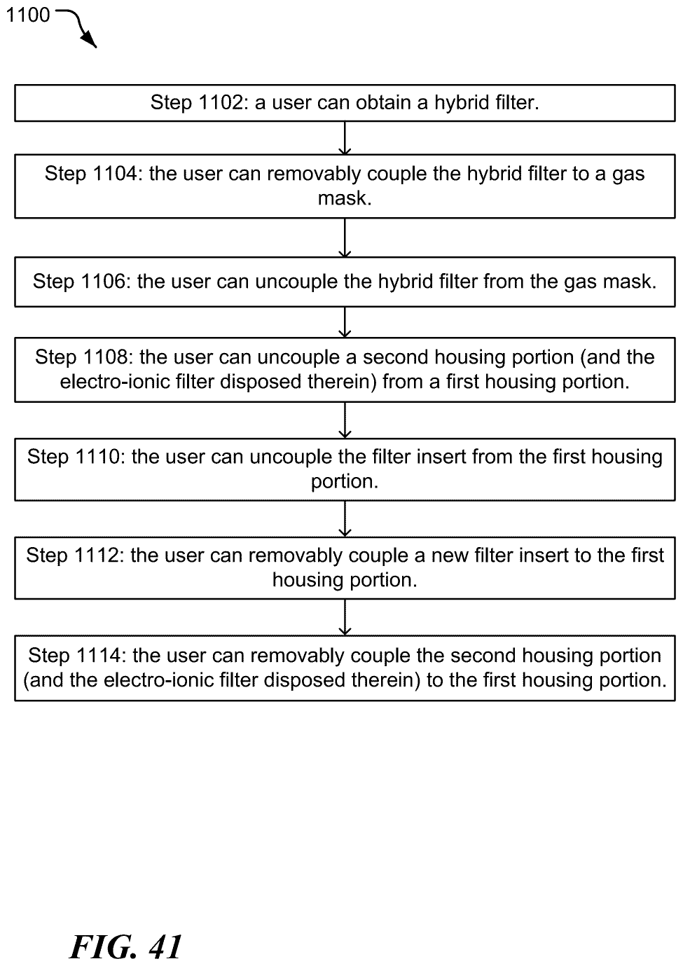

Turning now to FIG. 41, an example method 1100 of field assembling a hybrid filter 400 is illustrated in a flowchart, according to one embodiment. Each block shown in FIG. 41 can represent one or more processes, methods, or subroutines, carried out in the example method 1100. Furthermore, the illustrated order of blocks is illustrative only and the order of the blocks can change according to the present disclosure. Additional blocks can be added or fewer blocks can be utilized, without departing from this disclosure.

At block 1102, the user can obtain a hybrid filter 400 (which can also be referred to as a modular filter), as illustrated for example in FIGS. 23-25. In some instances, the user is a military service member (e.g., soldier, marine). The hybrid filter 400 can be in a kit (e.g., preassembled hybrid filter 400, disassembled hybrid filter 400). In some examples, the hybrid filter 400 is preassembled in the kit. In a preassembled state, the first housing portion (e.g., housing cap 49), which has a filter insert 408 removably coupled therein, is removably coupled to the second housing portion (e.g., housing 50), which has an electro-ionic filter 10′ disposed therein. When the hybrid filter 400 is preassembled, the user can proceed to block 1104. In other examples, the hybrid filter 400 is disassembled in the kit (e.g., the first housing portion is not coupled to the second housing portion). When the hybrid filter 400 is disassembled, the user can assemble the hybrid filter 400 as described below, such as at blocks 1112-1114.

At block 1104, the user can removably couple (e.g., threadably mate) the hybrid filter 400 (as illustrated for example in FIGS. 23-25) to a mask 20 (as illustrated for example in FIG. 1). The user can power on the hybrid filter 400 such that the electro-ionic filter 10′ provides filtration. The user can respirate, breathing in through the hybrid filter 400 such that inhalation air travels through the electro-ionic filter 10′ and through the filter insert 408 (and activated carbon-based filter 25 disposed therein). In some examples, the electro-ionic filter 10′ (or the ozone generator) can produce a relatively higher level of ozone (e.g., first concentration of ozone) when the hybrid filter 400 is initially turned on, which can purge contaminants within the hybrid filter 400 (e.g., activated carbon-based filter 25). After the purge, the electro-ionic filter 10′ can produce a relatively lower level of ozone (e.g., second concentration of ozone).

At block 1106, the user can uncouple (e.g., unthread) the hybrid filter 400 (as illustrated for example in FIGS. 23-25) from the mask 20 (as illustrated for example in FIG. 1) such as, for example, when one or more components of the hybrid filter 400 is at or near the end of its service life. In some examples, the activated carbon-based filter 25 within the filter insert 408 is at or near the end of its service life. The user can service (or otherwise remove and replace) one or more components of the hybrid filter 400 when it is uncoupled from the mask 20.

At block 1108, the user can uncouple (e.g., unthread) a second housing portion (e.g., housing 50), which includes an electro-ionic filter 10′ disposed therein, from a first housing portion (e.g., housing cap 49). Then, the user can separate the second housing portion from the first housing portion, which includes a filter insert 408 removably coupled therein. This allows the user to access the filter insert 408 within the first housing portion.

At block 1110, the user can uncouple (e.g., unthread) the filter insert 408 (e.g., a used filter insert 408 that is at or near the end of its service life) from the first housing portion (e.g., housing cap 49). The filter insert 408 can be separated from the first housing portion and discarded.

At block 1112, the user can removably couple (e.g., threadably mate) a filter insert 408 (e.g., a new filter insert 408, a filter insert 408 having remaining service life) to the first housing portion (e.g., housing cap 49).

At block 1114, the user can removably couple (e.g., threadably mate) the second housing portion (e.g., housing 50), which includes an electro-ionic filter 10′ disposed therein, to the first housing portion (e.g., housing cap 49). Then, the user can removably couple (e.g., threadably mate) the hybrid filter 400 (as illustrated for example in FIGS. 23-25) to the mask 20 (as illustrated for example in FIG. 1), as previously discussed at block 1102.

In some embodiments, the electro-ionic filter 10′ (e.g., used electro-ionic filter 10′ that is at or near the end of its service life), and the second housing portion within which the electro-ionic filter 10′ is disposed, can separated from the first housing portion and discarded. Then, the user can removably couple (e.g., threadably mate) a second housing portion having an electro-ionic filter 10′ therein (e.g., new electro-ionic filter 10′, electro-ionic filter 10′ having remaining service life) to the first housing portion.

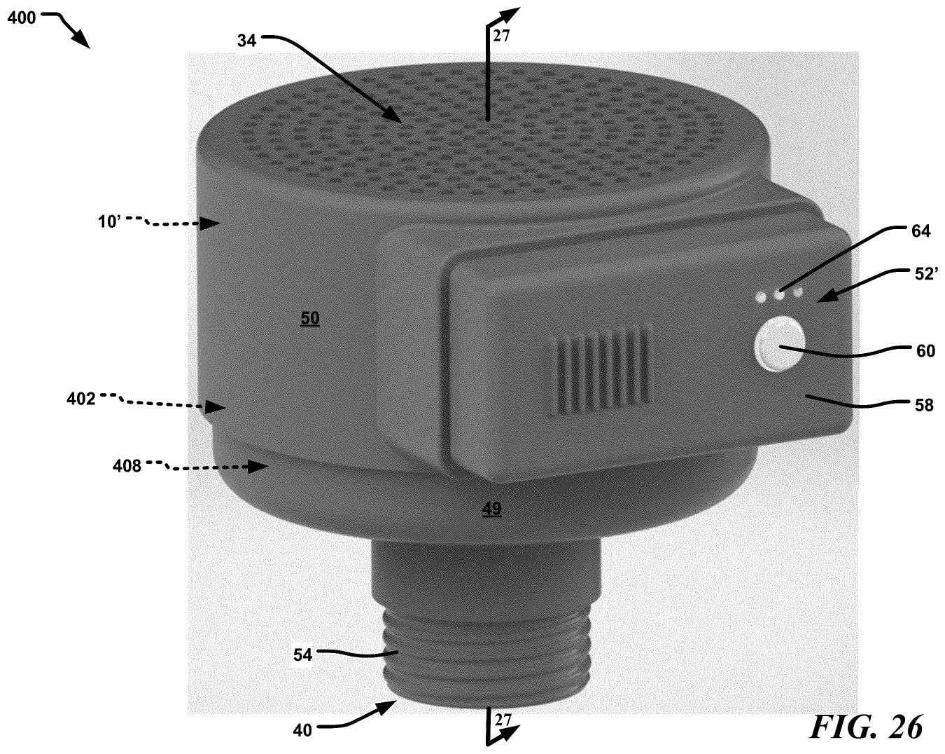

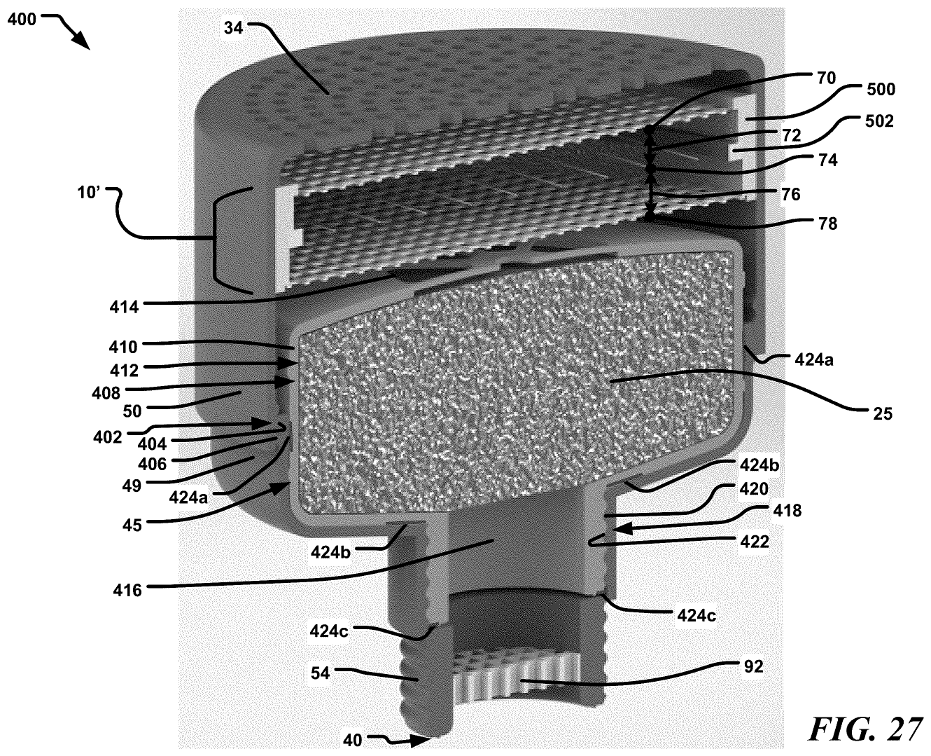

Turning to FIGS. 26-28, the hybrid filter 400 is illustrated. A ozone scrubber 92 (e.g., manganese oxide filter) extends across the opening of the airflow exit 40, as illustrated for example in FIG. 27. In some aspects, the ozone scrubber 92 is an ozone catalytic scrubber including an MnO grid coating that reduces O₃ to O₂. In this manner, the ozone scrubber 92 can protect the lungs from elevated levels of ozone within the hybrid filter 400. As previously discussed, the elevated levels of ozone within the hybrid filter can degrade captured chemical agents and regenerate the activated charcoal filter.

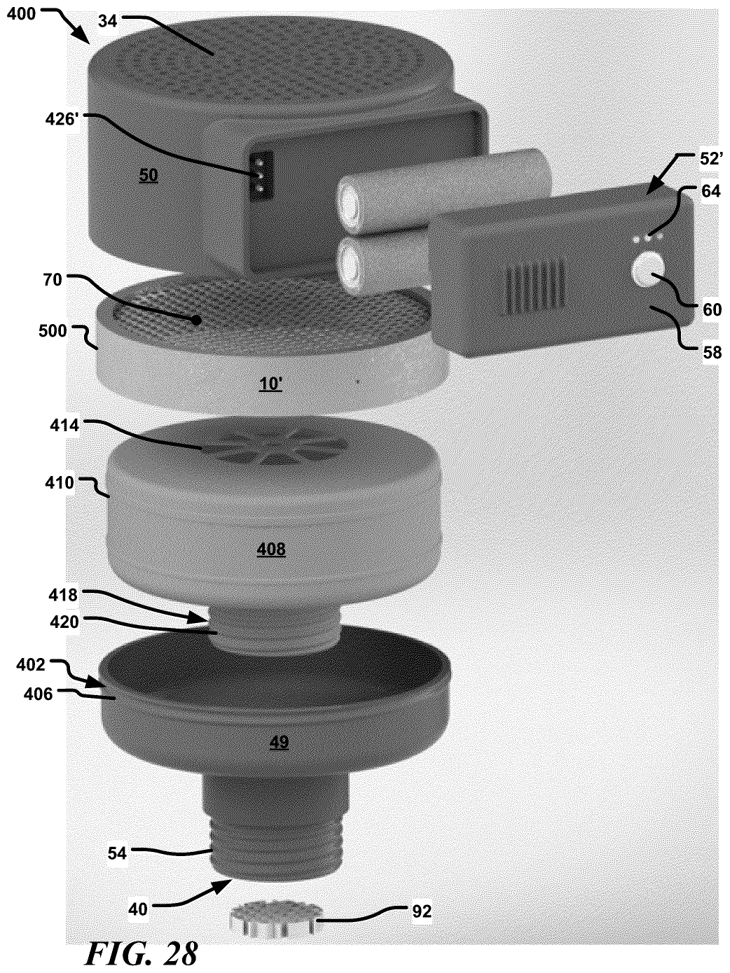

The electronics box 52′ includes removable batteries, as illustrated in FIG. 28, that are enclosed by the electronics box 52′ when the electronics box is removably coupled to the housing 50. The hybrid filter 400 is shown in a bottom-side perspective view (in FIG. 26), a cross-sectional perspective view as taken along section line 27-27 in FIG. 26 (in FIG. 27), and an exploded view (in FIG. 28). The electronics box 52′ can include a connection terminal 426′ having an external plug (or external connectors). In some aspects, the connection terminal 426′ (e.g., triple connector) can be used to receive power from the battery (or batteries) and also to receive power from an external power source (e.g., wall outlet).

Turning now to FIGS. 29-30, an electro-ionic filter 10′ is illustrated. The electro-ionic filter 10′ (as illustrated for example in FIGS. 29-30) can include one or more same or similar features, components, or elements as the electro-ionic filter 10 (as illustrated for example in FIGS. 1-16 and as previously described). The electro-ionic filter 10′ is illustrated in an assembled view (in FIG. 29) and in an exploded view (in FIG. 30).

The electro-ionic filter 10′ includes a positive electrode 70 (the first electrode), a negative electrode 74 (the second electrode), and a positive electrode 78 (the third electrode). That is, the three electrode arrangement is a positive-negative-positive (P-N-P) arrangement. As illustrated for example in FIG. 24 and FIG. 27, the first electrode 70 and the second electrode 74 define a first offset space 72 therebetween, and the second electrode 74 and the third electrode 78 define a second offset space 76 therebetween.

The electro-ionic filter 10′ includes a mounting ring 500 and an insulating ring 502. The mounting ring 500 is constructed of a conductive material, such as metal, such that the mounting ring 500 can form the sidewalls of the Faraday cage. In turn, the Faraday cage isolates the electrical environment produced by the electro-ionic filter 10′. The insulating ring 502 is disposed within the mounting ring 500 such that the longitudinal axis of the insulating ring 502 is coaxial with the longitudinal axis of the mounting ring 500 when the electro-ionic filter 10′ is assembled, as illustrated for example in FIG. 24 and FIG. 27. The outer diameter of the insulating ring 502 corresponds to the inner diameter of the mounting ring 500. The insulating ring 502 is constructed of an insulated material, such as plastic. The mounting ring 500 supports each positive electrode 70, 78 and the insulating ring 502 supports the negative electrode 74. In this manner, the negative electrode 74 is electrically insulated from the mounting ring 500, which functions as a part of the Faraday cage as described above.

Continuing with FIGS. 29-30, each positive electrode 70, 78 (also referred to as the collector electrodes) includes a grid/grille configuration 83 that is substantially perpendicular to the longitudinal axis of the mounting ring 500. The negative electrode 74 (also referred to as the emitter electrode) includes a grid/grille configuration 83′ that is substantially perpendicular to the longitudinal axis of the insulating ring 502. Both the grid/grille configuration 83 and the grid/grille configuration 83′ allow air to flow therethrough (e.g., airflow along the direction of the longitudinal axis of the mounting ring 500). The grid/grille configuration 83 of each positive electrode 70, 78 includes less open area for airflow as compared to the grid/grille configuration 83′ of the negative electrode 74. Correspondingly, the grid/grille configuration 83 of each positive electrode 70, 78 includes more metal making up the thin metal components 85 of the grid/grille configuration 83 as compared to the thin metal components 85′ of the grid/grille configuration 83′ of the negative electrode 74).

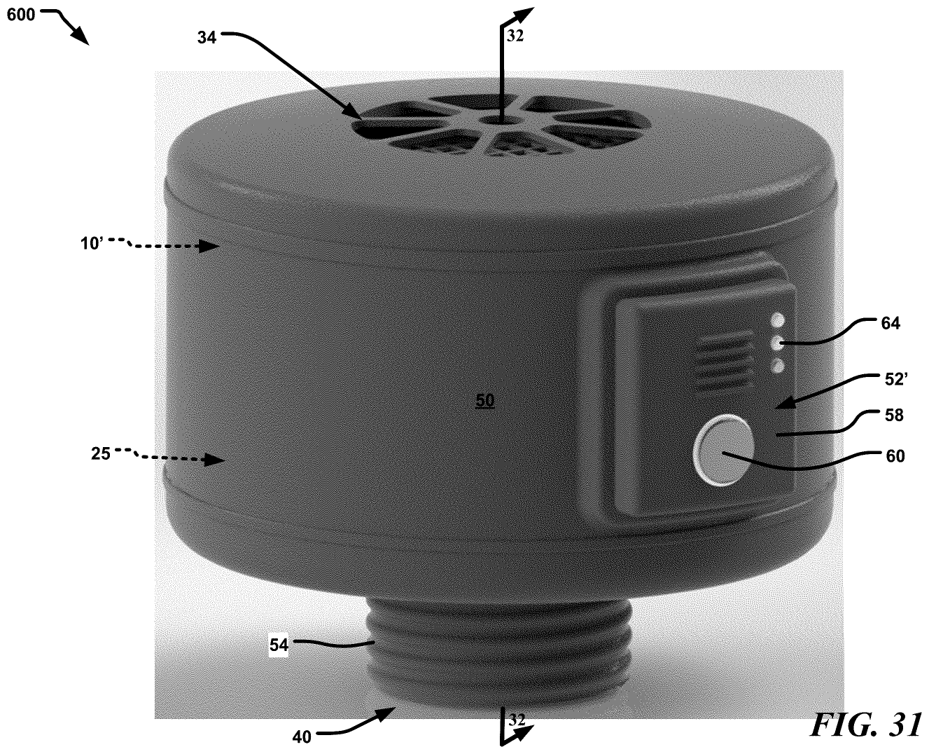

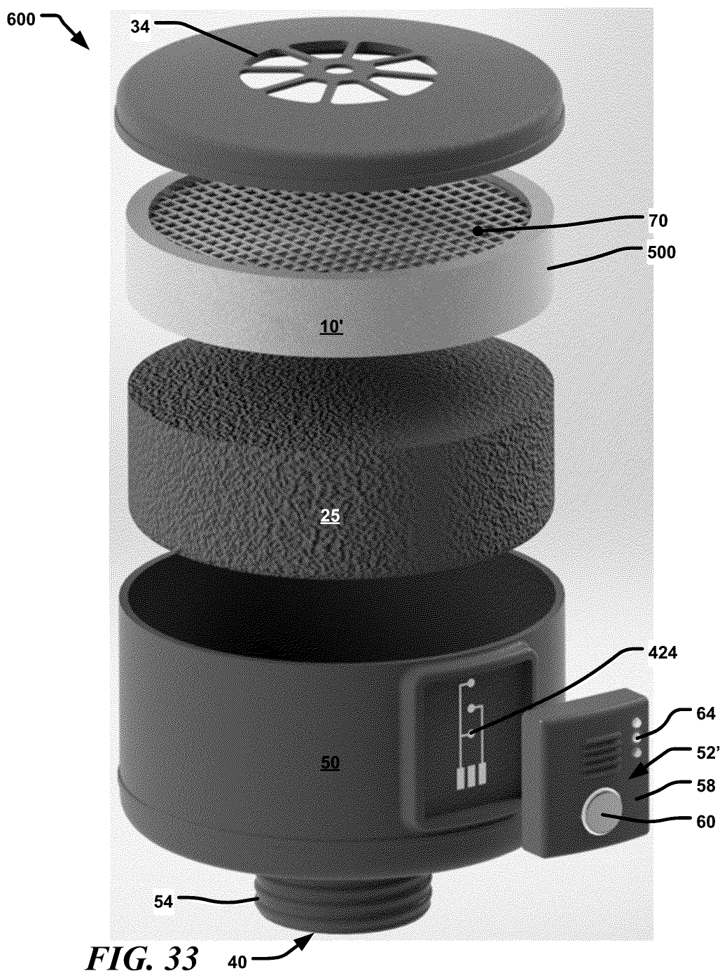

Turning now to FIGS. 31-33, a hybrid filter 600 is illustrated. The hybrid filter 600 includes an electro-ionic filter 10′ and an activated carbon-based filter 25 oriented in series therein. The hybrid filter 600 is shown in a bottom-side perspective view (in FIG. 31), in a cross-sectional perspective view as taken along section line 32-32 in FIG. 31 (in FIG. 32), and in an exploded view (in FIG. 33). Without departing from the teaching in the present disclosure, the various components of the hybrid filter 600 can be combined into a different arrangement of components, and the hybrid filter 600 can include more or less components without limitation.

The hybrid filter 600 (as illustrated for example in FIGS. 31-33) can be used in a same or similar manner as the hybrid filter 30 (as illustrated for example in FIGS. 1-16 and as previously described), the filter 200 (as illustrated for example in FIGS. 17-20 and as previously described), and/or the hybrid filter 400 (as illustrated for example in FIGS. 23-28 and as previously described). For example, the hybrid filter 600 (as illustrated for example in FIGS. 31-33) can include one or more same or similar features, components, or elements as the hybrid filter 30 (as illustrated for example in FIGS. 1-16), the filter 200 (as illustrated for example in FIGS. 17-20), and/or the hybrid filter 400 (as illustrated for example in FIGS. 23-28). Moreover, the hybrid filter 600 can employ a method that is the same as or similar to the method 300 (as illustrated for example in FIG. 22 and as previously discussed).

The hybrid filter 600 includes an electro-ionic filter 10′ upstream of an activated carbon-based filter 25 (e.g., activated carbon filter). In this manner, airflow through the hybrid filter 600 is drawn through the electro-ionic filter 10′ and then the activated carbon-based filter 25. The hybrid filter 600 includes a continuous housing 50. That is, the housing 50 does not include a removable housing cap. In this manner, the internal components of the hybrid filter 600 are integrated therein. Said another way, the housing 50 (or canister) is integrated contains an integrated electro-ionic filter 10′. The power module supplies a high voltage to the electro-ionic filter 10′. In some embodiments, the power module is removably coupled to the housing 50.

Continuing with FIGS. 31-33, one or more gaskets 602 (e.g., gasket 602a, gasket 602b) can be included at the joint between the activated carbon-based filter 25 and the chamber 45 (e.g., housing cap 49), as illustrated for example in the cross-sectional view of FIG. 32. In some examples, the gasket 602 is an o-ring. When present, the gasket 602 impedes, or otherwise prevents, air from flowing through the joint. In this manner, the gasket 602 prevents airflow from short-circuiting the activated carbon-based filter 25. Said another way, the gasket 602 causes air to flow through the activated carbon-based filter 25. In some examples, the gasket 602 is constructed of polymer (e.g., rubber). In some embodiments, the one or more gaskets 602 are integrated into the manufacture of the housing cap 49.

The position of the gaskets 602 as illustrated in FIG. 32 should not be construed as limiting. Nevertheless, as illustrated for example in FIG. 32, a gasket 602a can be positioned between a sidewall of the chamber 45 (e.g., inner circumferential surface of the housing cap 49) and a corresponding sidewall of the activated carbon-based filter 25 (e.g., outer circumferential surface of the activated carbon-based filter 25). Additionally or alternatively, a gasket 602b can be positioned between a base of the chamber 45 (e.g., base of the housing cap 49) and a corresponding base of the activated carbon-based filter 25.

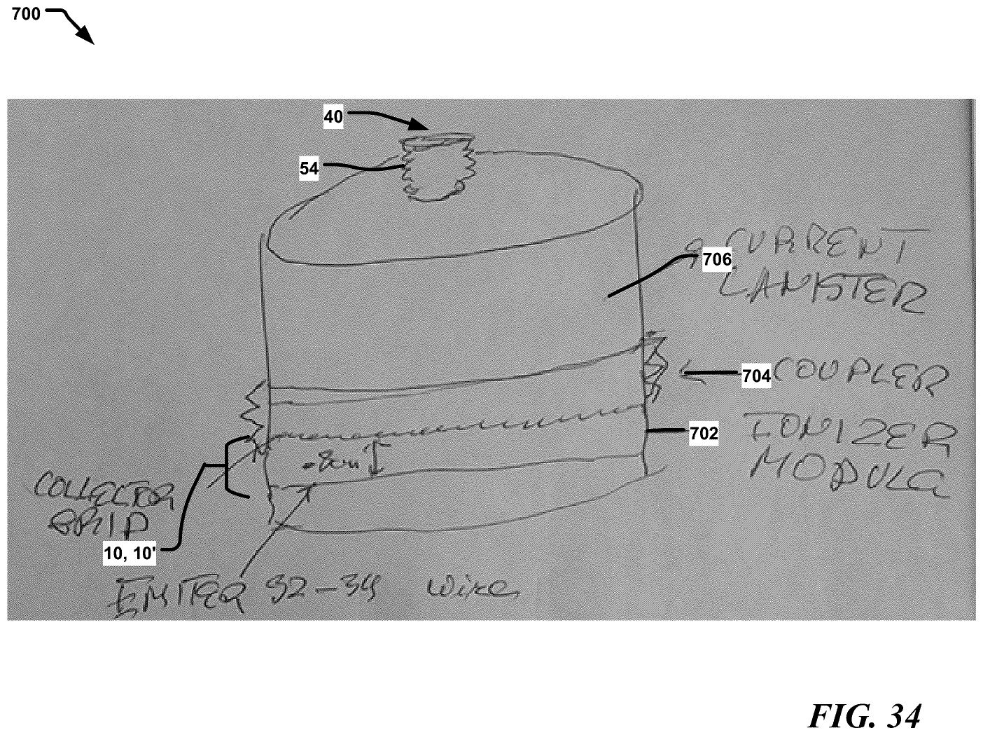

Turning now to FIG. 34, a hybrid filter 700 is illustrated. The hybrid filter 700 is illustrated in a bottom-side perspective view (FIG. 34). The hybrid filter 700 includes an ionization module 702 that has an electro-ionic filter (e.g., electro-ionic filter 10, electro-ionic filter 10′) disposed therein. The ionization module 702 includes a housing that can be removably coupled to the housing of an existing canister 706 (e.g., an existing standard 40 mm canister) via an attachment mechanism 704 (also referred to as a coupler). In this manner, the hybrid filter 700 is modular in that the ionization module 702 (e.g., electro-ionic filter 10) is interchangeable.

The attachment mechanism 704 can include threads, latches, bayonet connectors, interference fits, a hinge and latch, or the like. In some embodiments, a gasket can be included at the joint between the ionization module 702 and housing 50 to impede, or otherwise prevent, air from flowing through the joint. In some embodiments, the attachment mechanism 704 includes one or more bolts configured to bolt the ionizer module 702 to canister.

When the ionizer module 702 is coupled to the intake end of the housing of an existing canister 706, the ionizer module 702 can enhance the functionality of the existing can existing canister 706. That is, during operation the ionizer module 702 can provide ionic filtration of airflow passing therethrough. In some instances, the ionizer module 702 can generate incidental ozone that can regenerate a carbon filter disposed within the housing of the existing canister 706. In this manner, the ionizer module 702 functions the same as similar as electro-ionic filters 10 described throughout this application but with respect to an existing cannister 706. The ionizer module 702 can be uncoupled (e.g., disconnected) and removed from the housing of the existing canister 706.

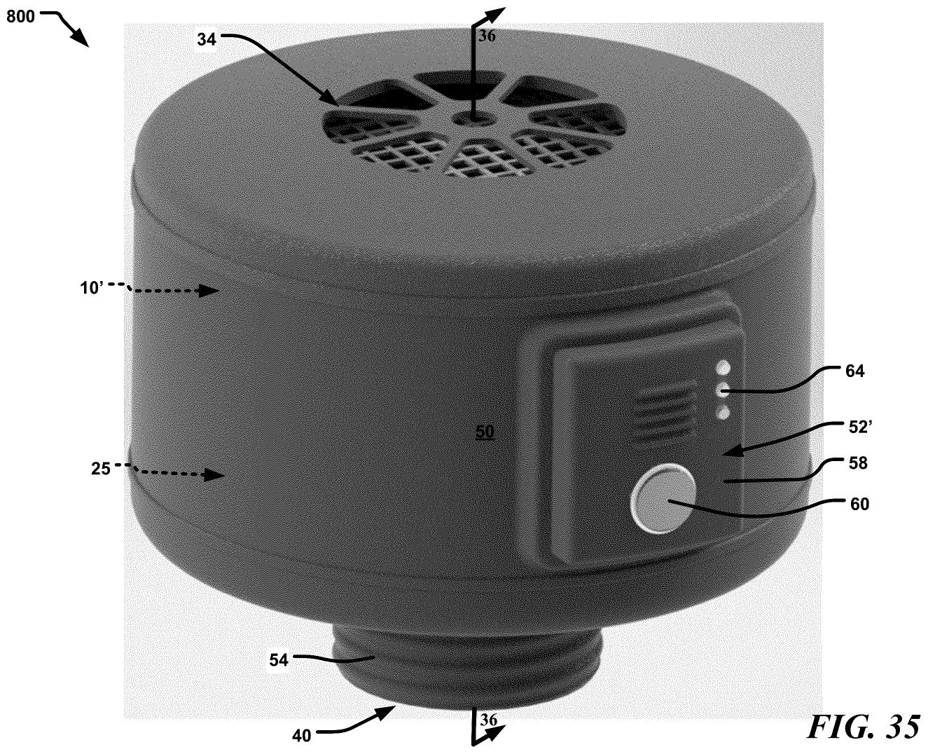

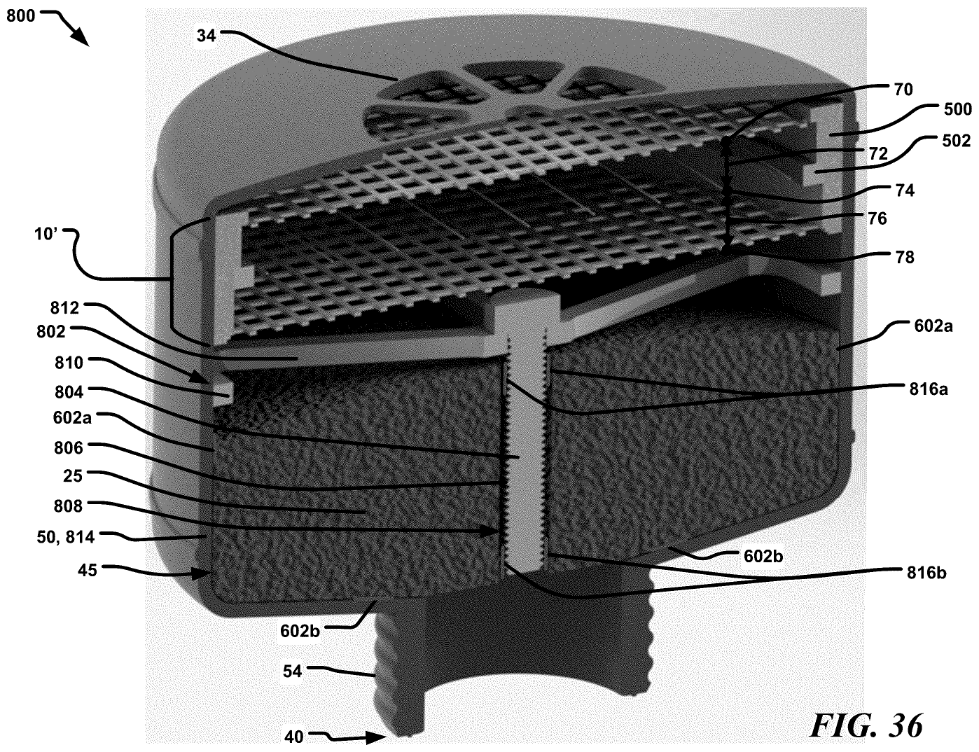

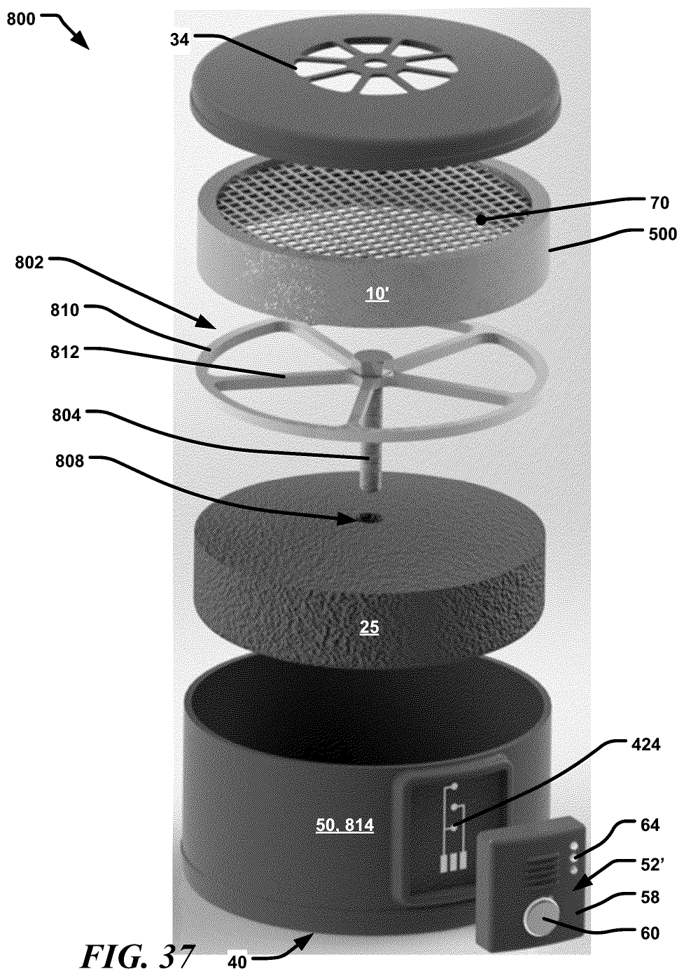

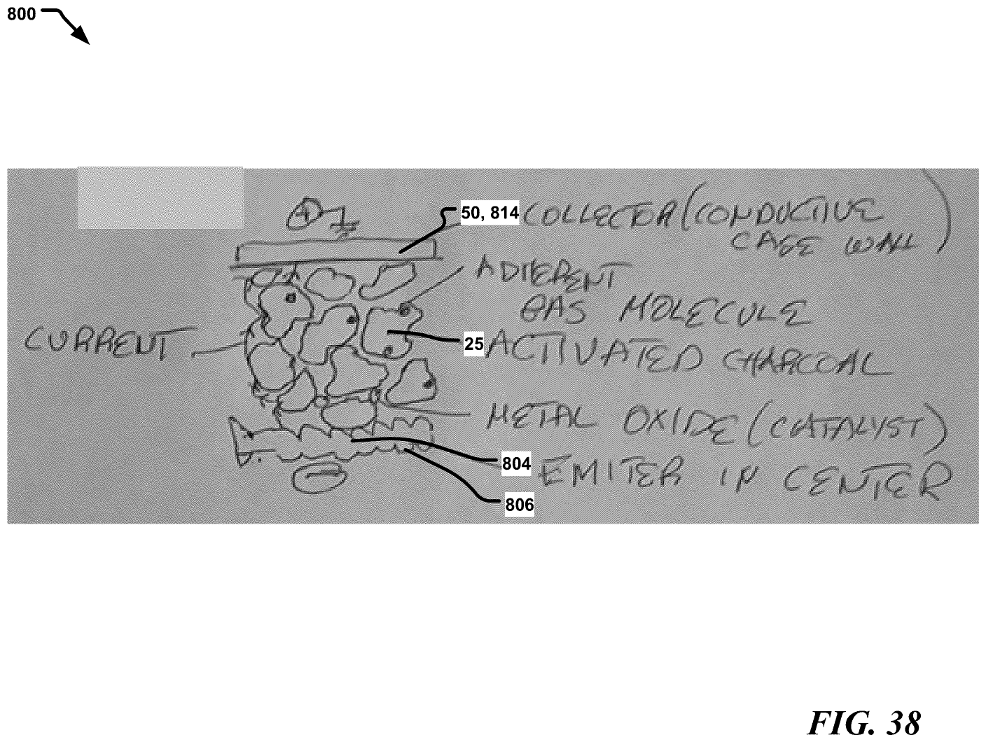

Turning now to FIGS. 35-38, a hybrid filter 800 is illustrated. The hybrid filter 800 includes an electro-ionic filter 10′ and an activated carbon-based filter 25 oriented in series therein. The hybrid filter 800 is shown in a bottom-side perspective view (in FIG. 35), in a cross-sectional perspective view as taken along section line 36-36 in FIG. 35 (in FIG. 36), in an exploded view (in FIG. 37), and in a cross-sectional view of the activated charcoal of the hybrid filter of FIG. 35 with current flow therethrough (FIG. 38). Without departing from the teaching in the present disclosure, the various components of the hybrid filter 800 can be combined into a different arrangement of components, and the hybrid filter 800 can include more or less components without limitation.

The hybrid filter 800 (as illustrated for example in FIGS. 35-38) can be used in a same or similar manner as the hybrid filter 30 (as illustrated for example in FIGS. 1-16 and as previously described), the filter 200 (as illustrated for example in FIGS. 17-20 and as previously described), the hybrid filter 400 (as illustrated for example in FIGS. 23-28 and as previously described), the hybrid filter 600 (as illustrated for example in FIGS. 31-33 and as previously described), and/or the filter 700 (as illustrated for example in FIG. 34 and as previously described). For example, the hybrid filter 800 (as illustrated for example in FIGS. 35-38) can include one or more same or similar features, components, or elements as the hybrid filter 30 (as illustrated for example in FIGS. 1-16), the filter 200 (as illustrated for example in FIGS. 17-20), the hybrid filter 400 (as illustrated for example in FIGS. 23-28), the hybrid filter 600 (as illustrated for example in FIGS. 31-33), and/or the filter 700 (as illustrated for example in FIG. 34). Moreover, the hybrid filter 800 can employ a method that is the same as or similar to the method 300 (as illustrated for example in FIG. 22 and as previously discussed).

Continuing with FIGS. 35-38, the hybrid filter 800 includes a dynamic energy pump 802 (also referred to as a conductive pump) disposed therein. During use, the energy pump 802 can dynamically decompose toxic gases that have been adsorbed by the activated carbon-based filter 25 (e.g., activated carbon filter). In turn, this regenerates (or otherwise refreshes) the activity of the carbon particles.

In some embodiments, the energy pump 802 can be used with ozone (e.g., ozone generated via the electro-ionic filter 10′, ozone generated via an ozone generator). In some examples, as illustrated for example in FIGS. 35-38, the hybrid filter 800 includes an electro-ionic filter 10′ disposed therein. In these examples, the power module can supply a high voltage to the electro-ionic filter 10′ and a low voltage to the energy pump 802. In some examples, the hybrid filter 800 includes an ozone generator (such as ozone generator 202 as illustrated for example in FIGS. 17-20) disposed therein.

In some embodiments, the energy pump 802 can be used without ozone generation. In some examples, the hybrid filter 800 does not include either an electro-ionic filter 10′ or an ozone generator. The energy pump 802 can directly facilitate the regeneration of the carbon surface without bathing the particles in ozone as discussed previously in this disclosure. In these examples, the power module can supply a low voltage to the energy pump 802. The power module can also provide current control.

Continuing with FIGS. 35-38, the energy pump 802 includes an emitter 804 and a collector 814, which, together, are configured to send a controlled current though the carbon particles of the activated carbon-based filter 25. In some embodiments, the emitter 804 is located within the carbon particles and the collector 814 is the encasing housing 50 (also referred to as the canister wall). In some aspects, a low voltage current (which can be either AC or DC) is sent through the compressed carbon particles, as illustrated for example in the cross-sectional view of FIG. 38. The current is primarily carried by the amorphous surface of each particle (site of toxin adsorption). In the presence of catalysts, the current becomes the energy pump that decomposes the adsorbed toxin molecules.

The current facilitates the oxidation of toxic molecules that are adherent to such carbon particle amorphous surface. This surface current flowing directly through the carbon particles becomes the dynamic energy pump, which accelerates various oxidative and catalytically facilitated reactions. In turn, a significant degree of canister absorptive regeneration capability (which can be the same or similar as the regeneration from bathing of such particles in active ozone) is achieved.

In some embodiments, as illustrated for example in FIGS. 35-38, the housing 50 serves as the collector 814. In some examples, the longitudinal axis of the emitter 804 is substantially parallel to (and in some cases, coaxial with) the longitudinal axis of the collector 814. In some examples, the emitter 804 extends longitudinally along the longitudinal axis (e.g., central axis) of the collector 814. In this manner, the collector 814 radially encompasses the emitter 804.

One or more gaskets 816 (e.g., gasket 816a, gasket 816b) can be included at the joint between the emitter 804 and the activated carbon-based filter 25, as illustrated for example in the cross-sectional view of FIG. 36. In some examples, the gasket 816 is an o-ring. When present, the gasket 816 impedes, or otherwise prevents, air from flowing through the joint. In this manner, the gasket 816 prevents airflow from short-circuiting the activated carbon-based filter 25. Said another way, the gasket 816 causes air to flow through activated carbon-based filter 25. In some examples, the gasket 816 is constructed of polymer (e.g., rubber). In some embodiments, the one or more gaskets 816 are integrated into the manufacture of the emitter 804.

The position of the gaskets 816 as illustrated in FIG. 36 should not be construed as limiting. Nevertheless, as illustrated for example in FIG. 36, one or more gaskets (e.g., gasket 816a, gasket 816b) can be positioned between a sidewall of the activated carbon-based filter 25 (e.g., inner circumferential surface of the activated carbon-based filter 25) and the emitter 804 (e.g., outer circumferential surface of the emitter 804). For example, a gasket 816a can be disposed around the emitter 804 at or near a first end of the activated carbon-based filter 25. Additionally or alternatively, a gasket 816b can be disposed around the emitter at or near a second end of the activated carbon-based filter 25 (e.g., at or near the airflow exit 40 of the hybrid filter 800).

Continuing with FIGS. 35-38, the emitter 804 can include one or more protrusions 806 (e.g., points, threads) extending radially outward therefrom. In some examples, the emitter 804 is a screw or bolt and the protrusions 806 are threads helically wrapped thereabout. In some embodiments, the emitter 804 is disposed within an opening 808 (e.g., cylindrical opening) in the material-based filtration element 85. In some examples, the activated carbon-based filter 25 encases the emitter 804.

A ring 810 having one or more arms 812 extending radially inward can support the emitter 804. The outer diameter of the ring 810 can correspond to the inner diameter of the housing 50. The ring 810 can be coupled to the housing 50 and the emitter 804 can be coupled to the arms 812 extending from the ring 810. In this manner, the position of the emitter 804 can be fixed relative to the position of the housing 50.

Continuing with FIGS. 35-38, the energy pump 802 uses significantly lower voltage(s) than the voltage(s) used for the electro-ionic filter 10′ or the ozone generator require to regenerate active carbon. This is because the packed carbon particles function as a semiconductor. In some embodiments, the operational voltage range of the energy pump is between 3 volts and 120 volts. The operational voltage can depend on whether a battery source is used or whether a system wall source is used during accelerated canister regeneration cycle.

In some aspects, the accelerated canister regeneration cycle is similar to the self-cleaning mode previously described. That is, the device can initiate an accelerated regeneration mode when the device is charging (e.g., plugged in to an electrical power supply). During the accelerated regeneration mode, the device can generate ozone at higher levels for a predetermined period of time (e.g., between approximately 5 minutes and approximately 10 minutes). The higher levels of ozone within the ozonated chamber burn off retained toxic chemicals. The ozone accumulates and regenerates used up activated charcoal.

The efficacy of the energy pump 802 (e.g., via direct current sent through the carbon particles) can be obtained by sending high voltage, low current, in DC form, AC form, or pulsating form. Sending such high voltage, low current through compressed particles of activated carbon can benefit from high frequency modulation (60 hz-4 mhz) because of increasing skin effect at surface of such particles. Lower frequencies can accomplish toxin decomposition via heating effects and higher frequencies via gap-ionization effects. In some embodiments, higher frequencies achieve toxin decomposition at lower energy cost. In some examples, a combination of low voltage, higher current and high voltage, high frequency, low current is sent through the canister carbon filler either continually or periodically during its use and between uses. In some aspects, the optimal signal combination of DC and HF AC or pulsing HF is determined by the field application and performance need.

Continuing with FIGS. 35-38, in addition to cleaning the carbon particles as previously described, the current flowing through the carbon can provide a measure the absorptive saturation of the carbon particles. The overall resistance will follow the percent of saturation and become the measure of residual life of the canister (e.g., remaining service life of the activated carbon-based filter 25 that includes activated charcoal). In some embodiments, the hybrid filter 800 includes external connectors (e.g., similar to connection terminal 426′ as illustrated for example in FIG. 28), which can connect to stronger current sources (e.g., wall current) than battery. In some examples, the hybrid filter 800 can provide tabletop canister regeneration after extensive use and measured loss of protective capacity by connecting to wall current (with current control) to essentially “burn off” the absorbed toxic molecules. Thus, the hybrid filter 800 can provide the means of measuring residual capacity and the means of regenerating the utility of such canisters in the absence of total replacement.

FIG. 39 illustrates a suitable computing and networking environment 900 (e.g., computing device 900), according to some embodiments of the present disclosure, that can be part of or useable with the system described herein. In other words, the computing device 900 can be used to implement various aspects of the present disclosure described herein. In some embodiments, the hybrid filter 30 (as illustrated for example in FIGS. 1-16), the filter 200 (as illustrated for example in FIGS. 17-20), the hybrid filter 400 (as illustrated for example in FIGS. 23-28), the hybrid filter 600 (as illustrated for example in FIGS. 31-33), the filter 700 (as illustrated for example in FIG. 34), and/or the hybrid filter 800 (as illustrated in FIGS. 35-38) can be operated with (or otherwise on) a computing device 900 (as illustrated for example in FIG. 39).

As illustrated, the computing and networking environment 900 includes a general purpose computing device 900, although it is contemplated that the networking environment 900 may include other computing systems, such as smart phones, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 900 may include various hardware components, such as a processing unit 902, a data storage 904 (e.g., a system memory), and a system bus 906 that couples various system components of the computer 900 to the processing unit 902. The system bus 906 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 900 may further include a variety of computer-readable media 908 that includes removable/non-removable media and volatile/nonvolatile media, but excludes transitory propagated signals. Computer-readable media 908 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 900. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 904 includes computer storage media in the form of volatile/nonvolatile memory such as read only memory (ROM) and random access memory (RAM). A basic input/output system (BIOS), containing the basic routines that help to transfer information between elements within the computer 900 (e.g., during start-up) is typically stored in ROM. RAM typically contains data and/or program modules that are immediately accessible to and/or presently being operated on by processing unit 902. For example, in one embodiment, data storage 904 holds an operating system, application programs, and other program modules and program data.

Data storage 904 may also include other removable/non-removable, volatile/nonvolatile computer storage media. For example, data storage 904 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 39, provide storage of computer-readable instructions, data structures, program modules and other data for the computer 900.

A user may enter commands and information through a user interface 910 or other input devices such as a tablet, electronic digitizer, a microphone, keyboard, and/or pointing device, commonly referred to as mouse, trackball or touch pad. The commands and information may be for setting up the electro-ionic filter 10 (as illustrated for example in FIGS. 1-16), the ozone generator 202 (as illustrated for example in FIGS. 17-20), the humidity source 204 (as illustrated for example in FIGS. 17-20), the electro-ionic filter 10′ (as illustrated for example in FIGS. 23-37) and/or the energy pump 802 (as illustrated for example in FIGS. 36-38) including the specific parameters of each. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 902 through a user interface 910 that is coupled to the system bus 906, but may be connected by other interface and bus structures, such as a parallel port, game port or a universal serial bus (USB).

The computer system 900 can include one or more ports, such as an input/output (I/O) port 912. The I/O port 912 can be connected to an I/O device, or other device, by which information is input to or output from the computing system 900. Such I/O devices may include, without limitation, one or more input devices, output devices, and/or environment transducer devices.

In some embodiments, the I/O port 912 is in communication with one or more sensors 916. The sensors 916 (as illustrated in FIG. 39) can include a toxin sensor (which can include same or similar components as toxin sensor 210 illustrated for example in FIGS. 18-21), an ozone sensor (which can include same or similar components as ozone sensor 212 illustrated for example in FIGS. 18-21), and/or a humidity sensor (which can include same or similar components as humidity sensor 214 illustrated for example in FIGS. 18-21).

In some embodiments, the I/O port 912 is in communication with one or more generators 918. The generators 918 (as illustrated in FIG. 39) can include an electro-ionic filter (which can include same or similar components as electro-ionic filter 10 illustrated for example in FIGS. 1-16 and/or electro-ionic filter 10′ illustrated for example in FIGS. 23-37), an ozone generator (which can include same or similar components as ozone generator 202 illustrated for example in FIGS. 17-20), a humidity source (which can include same or similar components as humidity source 204 illustrated for example in FIGS. 17-20), and/or an energy pump (which can include same or similar components as energy pump 802 illustrated for example in FIGS. 36-38).

The computer 900 may operate in a networked or cloud-computing environment using a communication module 914 (e.g., logical connections of a network interface or adapter) to one or more remote devices, such as a remote computer. The remote computer may be a personal computer, a server, a router, a network PC, a peer device or other common network node, and typically includes many or all of the elements described above relative to the computer 900. The logical connections depicted in FIG. 39 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 900 may be connected to a public and/or private network through the communication module 914. In such embodiments, a modem or other means for establishing communications over the network is connected to the system bus 906 via the communication module 914 or other appropriate mechanism. A wireless networking component including an interface and antenna may be coupled through a suitable device such as an access point or peer computer to a network. In a networked environment, program modules depicted relative to the computer 900, or portions thereof, may be stored in the remote memory storage device.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” and “approximately” and variations thereof as used herein when referring to a measurable value such as an amount, a temporal duration, and the like, is meant to encompass variations of +20%, +10%, +5%, +1%, and +0.1% from the specified value, as such variations are appropriate.

As used herein, the term “in communication with” can include a wired connection (e.g., Universal Serial Bus, Ethernet) or wireless connection (e.g., WiFi, Bluetooth).

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Illustrative Aspects of the Present Disclosure

Aspect 1-1. A hybrid filter for use with an activated carbon-based filter and attachable to a mask, the hybrid filter including: a chamber configured to receive the activated carbon-based filter; an electro-ionic filter in series with the chamber and including a plurality of electrodes arranged in series with each other, the electrodes of the plurality of electrodes being evenly spaced apart from each other in an alternating positive-polarity negative-polarity sequence; and a Faraday cage surrounding at least the electro-ionic filter.

Aspect 1-2. The hybrid filter of Aspect 1-1, wherein a longitudinal axis of the chamber is coaxial with a longitudinal axis of the electro-ionic filter.

Aspect 1-3. The hybrid filter of Aspect 1-1, wherein the chamber and the electro-ionic filter are disposed in a housing, the housing forming at least a portion of the Faraday cage.

Aspect 1-4. The hybrid filter of Aspect 1-3, wherein the housing includes at least one of a metal or a polymer coated with a conductive coating.

Aspect 1-5. The hybrid filter of Aspect 1-1, wherein the chamber and the electro-ionic filter are disposed in a housing, wherein the housing includes a neck configured to threadably couple to a port of the mask.

Aspect 1-6. The hybrid filter of Aspect 1-1, wherein the chamber and the electro-ionic filter are disposed in a housing, wherein an intake grille is disposed at a first end of the housing and an exit grille is disposed at a second end of the housing, the intake grille and the exit grille forming at least a portion of the Faraday cage.

Aspect 1-7. The hybrid filter of Aspect 1-6, wherein a longitudinal axis of the intake grille is colinear with a longitudinal axis of the exit grille.

Aspect 1-8. The hybrid filter of Aspect 1-1, wherein the chamber is further configured to also receive a particulate filter such that the particulate filter would be in series with the activated carbon-based filter.

Aspect 1-9. The hybrid filter of Aspect 1-1, further including an ozone scrubber in series with the chamber.

Aspect 1-10. The hybrid filter of Aspect 1-1, further including the activated carbon-based filter, which is configured to retain organic compounds, and wherein the electro-ionic filter is configured to generate ozone at a level that degrades the organic compounds via oxidative degradation.

Aspect 1-11. The hybrid filter of Aspect 1-1, further including the activated carbon-based filter, which has an absorptive capacity, and wherein the electro-ionic filter is configured to regenerate the absorptive capacity by controlled oxidation at ambient temperature.

Aspect 1-12. The hybrid filter of Aspect 1-1, wherein a concurrent operating voltage of the plurality of electrodes is at least one of: between approximately 2 kV and approximately 18 kV, between approximately 5 kV and approximately 15 kV, or between approximately 8 kV and approximately 12 kV.

Aspect 1-13. The hybrid filter of Aspect 1-1, wherein an applied voltage range between a pair of the plurality of electrodes is between approximately 1 kV and approximately 8 kV.

Aspect 1-14. The hybrid filter of Aspect 1-1, wherein the plurality of electrodes are arranged in a P-N-P, P-N-P-N-P, or P-N-P-N-P-N-P arrangement.

Aspect 1-15. The hybrid filter of Aspect 1-1, wherein the plurality of electrodes includes a first electrode module and a second electrode module, the first electrode module arranged in a P-N-P arrangement and the second electrode module arranged in a P-N-P arrangement.

Aspect 1-16. The hybrid filter of Aspect 1-1, wherein the plurality of electrodes includes at least one positive electrode arranged as a grid defining an open area for airflow therethrough and at least one negative electrode defining an open area for airflow therethrough, wherein the open area defined by the at least one negative electrode is greater than the open area defined by the at least one positive electrode.

Aspect 1-17. The hybrid filter of Aspect 1-1, wherein the plurality of electrodes includes at least one positive electrode and at least one negative electrode, the at least one positive electrode including wire arranged in a grid and defining a diameter, the at least one negative electrode including wire arranged in a grid and defining a diameter, wherein the diameter of the wire of the at least one positive electrode is greater than the diameter of the wire of the at least one negative electrode.

Aspect 1-18. The hybrid filter of Aspect 1-1, wherein each of the plurality of electrodes includes an open grid extending across a chamber and perpendicular to sidewalls of a housing containing the electro-ionic filter therein.

Aspect 1-19. The hybrid filter of Aspect 1-1, wherein the plurality of electrodes includes at least a starting positive electrode, an ending positive electrode, and at least one negative electrode disposed between the starting positive electrode and the ending positive electrode, wherein the starting positive electrode and the ending positive electrode are end portions of the Faraday cage encapsulating the at least one negative electrode.

Aspect 1-20. The hybrid filter of Aspect 1-1, wherein the plurality of electrodes includes at least one positive electrode acting as a positive collector and at least one negative electrode acting as a negative emitter.

Aspect 1-21. The hybrid filter of Aspect 1-1, wherein the plurality of electrodes are flat extending across the electro-ionic filter.

Aspect 1-22. The hybrid filter of Aspect 1-1, wherein the plurality of electrodes are curved extending across the electro-ionic filter.

Aspect 1-23. The hybrid filter of Aspect 1-1, wherein each of the plurality of electrodes includes an occluded portion and a non-occluded portion configured such that airflow is circuitously routed through the electro-ionic filter.

Aspect 1-24. The hybrid filter of Aspect 1-1, wherein a spacer section is disposed between each offset pair of the plurality of electrodes, each spacer section having an outer surface abutting an inner surface of a housing, wherein the electro-ionic filter is disposed in the housing.

Aspect 2-1. A hybrid filter for use with an activated carbon-based filter and attachable to a mask, the hybrid filter including: a chamber configured to receive the activated carbon-based filter; an electro-ionic filter in series with the chamber and including a plurality of electrode modules arranged in parallel with each other, each electrode module including a cylindrical positive collector electrode encompassing a negative emitter electrode extending coaxial with a longitudinal axis of the cylindrical positive collector electrode and equally spaced-apart from an inner cylindrical surface of the cylindrical positive collector electrode in all radial directions; and a Faraday cage surrounding at least the electro-ionic filter.

Aspect 2-2. The hybrid filter of Aspect 2-1, wherein the longitudinal axis of the cylindrical positive collector electrode is substantially parallel to a longitudinal axis of the chamber.

Aspect 2-3. The hybrid filter of Aspect 2-1, wherein the chamber is configured to also receive a particulate filter such that the particulate filter would be in series with the activated carbon-based filter.

Aspect 2-4. The hybrid filter of Aspect 2-1, further including an ozone scrubber in series with the chamber.

Aspect 2-5. The hybrid filter of Aspect 2-1, further including the activated carbon-based filter, which is configured to retain organic compounds, and wherein the electro-ionic filter is configured to generate ozone at a level that degrades the organic compounds via oxidative degradation.

Aspect 2-6. The hybrid filter of Aspect 2-1, further including the activated carbon-based filter, which has an absorptive capacity, and wherein the electro-ionic filter is configured to regenerate the absorptive capacity by controlled oxidation at ambient temperature.

Aspect 3-1. A filter for use with an activated carbon-based filter and attachable to a mask, the filter including: an ozonated chamber defined between an ozone generator and an ozone scrubber, the ozone generator configured to deliver a concentration of ozone into the ozonated chamber, the ozone scrubber configured to reduce the concentration of ozone leaving the ozonated chamber, the ozonated chamber configured to receive the activated carbon-based filter.

Aspect 3-2. The filter of Aspect 3-1, further including the activated carbon-based filter, which is configured to retain organic compounds passing through the ozonated chamber, and wherein the ozone generator is configured to deliver a concentration of ozone that degrades the organic compounds by oxidative degradation.

Aspect 3-3. The filter of Aspect 3-1, further including the activated carbon-based filter, which has an absorptive capacity, wherein the ozone generator is configured to deliver a concentration of ozone that regenerates the absorptive capacity by controlled oxidation at ambient temperature.

Aspect 3-4. The filter of Aspect 3-1, wherein the ozone generator includes one or more electrodes configured to generate ozone.

Aspect 3-5. The filter of Aspect 3-1, wherein the ozone generator is coupled to a housing that defines at least a portion of the ozonated chamber.

Aspect 3-6. The filter of Aspect 3-1, further including a humidity source configured to deliver humidity into the ozonated chamber.

Aspect 3-7. The filter of Aspect 3-1, further including a humidity source disposed within the ozonated chamber.

Aspect 3-8. The filter of Aspect 3-1, further including the activated carbon-based filter, which includes at least one of water or a buffering agent.

Aspect 4-1. A filter attachable to a mask, the filter including: an ozone generator configured to deliver ozone into an airflow at a first level of ozone; an activated carbon-based filter in series with the ozone generator and configured to capture contaminates from the airflow, the activated carbon-based filter exposed to the ozone within the airflow; and an ozone scrubber in series with the activated carbon-based filter and configured to reduce the ozone in the airflow to a second level of ozone, the second level of ozone being less than the first level of ozone.

Aspect 4-2. The filter of Aspect 4-1, wherein the first level of ozone causes oxidation of at least a portion of the contaminates that are captured.

Aspect 4-3. The filter of Aspect 4-1, wherein the first level of ozone is greater than 0.2 ppm.

Aspect 4-4. The filter of Aspect 4-1, wherein the ozone scrubber decomposes at least a portion of the ozone within the airflow.

Aspect 4-5. The filter of Aspect 4-1, wherein the second level of ozone is less than 0.1 ppm.

Aspect 5-1. A filter attachable to a mask, the filter including: a housing defining a chamber configured to receive airflow therethrough; an ozone generator coupled to the housing and configured to deliver ozone into the airflow; an activated carbon-based filter disposed within the chamber and configured to capture at least a portion of contaminates within the airflow, the activated carbon-based filter being downstream from the ozone generator; and an ozone scrubber configured to decompose at least a portion of the ozone in the airflow, the ozone scrubber being downstream from the activated carbon-based filter.

Aspect 5-2. The filter of Aspect 5-1, wherein the ozone generator generates a level of ozone to neutralize organophosphates.

Aspect 5-3. The filter of Aspect 5-1, wherein the ozone generator generates a level of ozone that oxidizes and degrades toxins retained by the activated carbon-based filter.

Aspect 5-4. The filter of Aspect 5-1, further including a particulate filter in series with the activated carbon-based filter.

Aspect 5-5. The filter of Aspect 5-1, wherein the ozone scrubber includes at least one of cobalt oxide or manganese oxide.

Aspect 5-6. The filter of Aspect 5-1, wherein the ozone generator includes an anode and a cathode.

Aspect 5-7. The filter of Aspect 5-1, wherein the ozone generator includes a positive electrode and a negative electrode.

Aspect 5-8. The filter of Aspect 5-1, wherein the activated carbon-based filter is impregnated with water.

Aspect 5-9. The filter of Aspect 5-1, further including a reservoir coupled to the housing, the reservoir configured to retain water such that the water humidifies the activated carbon-based filter.

Aspect 5-10. The filter of Aspect 5-1, wherein the activated carbon-based filter includes a pH buffering agent.

Aspect 6-1. A hybrid filter including: an activated carbon-based filter; an emitter disposed within the activated carbon-based filter; and a collector encompassing the emitter, wherein the emitter and collector are configured to provide a current through the activated carbon-based filter.

Aspect 6-2. The hybrid filter of Aspect 6-1, wherein the collector is a housing of the hybrid filter.

Aspect 6-3. The hybrid filter of Aspect 6-1, wherein the collector radially surrounds the emitter.

Aspect 6-4. The hybrid filter of Aspect 6-1, wherein the emitter is longitudinally disposed along a central axis of the hybrid filter.

Aspect 6-5. The hybrid filter of Aspect 6-1, further including an electro-ionic filter, wherein the activated carbon-based filter is configured to retain organic compounds, and wherein the electro-ionic filter is configured to generate ozone at a level that degrades the organic compounds via oxidative degradation.

Aspect 6-6. The hybrid filter of Aspect 6-1, further including an electro-ionic filter in series with the activated carbon-based filter.

Aspect 7-1. A hybrid filter attachable to a mask and for use with a filter insert including an inlet to receive airflow and an outlet to discharge the airflow, the filter insert including an activated carbon-based filter disposed therein, the hybrid filter including: a housing; a housing cap removably couplable to the housing; a chamber configured to receive the filter insert, wherein the filter insert would be removably couplable to at least one of the housing or the housing cap; and at least one of an electro-ionic filter or ozone generator in series with the chamber.

Aspect 7-2. The hybrid filter of Aspect 7-1, wherein the housing includes an air inlet and the housing cap includes an air outlet.

Aspect 7-3. The hybrid filter of Aspect 7-1, wherein the housing and the housing cap each contain corresponding threads, wherein the housing cap is removably coupled to the housing when the corresponding threads are rotatably mated together.

Aspect 7-4. The hybrid filter of Aspect 7-1, wherein the filter insert and the housing cap each contain corresponding threads, wherein the filter insert is removably coupled to the housing cap when the corresponding threads are rotatably mated together.

Aspect 7-5. The hybrid filter of Aspect 7-1, wherein the electro-ionic filter includes an emitter at least partially surrounded by a collector.

Aspect 7-6. The hybrid filter of Aspect 7-1, wherein the filter insert is manufactured by Avon protection or an equivalent.

Aspect 7-7. The hybrid filter of Aspect 7-1, wherein the filter insert includes at least one of a CBRNCF50 Filter, a FM61EU CBRN Filter, a GPCF50 CBRN Filter, a CFP100 Particulate Filter, or a CTCF50 Riot Agent Filter.

Aspect 7-8. The hybrid filter of Aspect 7-1, further including a gasket disposed between at least one of the filter insert and the housing or the filter insert and the housing cap when the filter insert is removably coupled thereto.

Aspect 8-1. A hybrid filter having a housing configured to removably receive a filter insert therein such that the filter insert is removably coupled to the housing, the filter insert having an inlet and a neck defining an outlet, the hybrid filter including: a first housing portion including a neck defining an airflow exit, the neck of the first housing portion configured to receive the neck of the filter insert therein such that the airflow exit of the first housing portion is coaxial with the outlet of the filter insert; a second housing portion defining an airflow inlet, the second housing portion configured to removably couple to the first housing portion; and at least one of an electro-ionic filter or ozone generator in series with the filter insert when the filter insert is removably coupled with the housing.

Aspect 8-2. The hybrid filter of Aspect 8-1, further including the filter insert.

Aspect 8-3. The hybrid filter of Aspect 8-2, wherein the filter insert includes an activated carbon-based filter disposed therein.

Aspect 8-4. The hybrid filter of Aspect 8-1, wherein the filter insert is manufactured by Avon protection or an equivalent.

Aspect 8-5. The hybrid filter of Aspect 8-1, wherein the filter insert includes at least one of a CBRNCF50 Filter, a FM61EU CBRN Filter, a GPCF50 CBRN Filter, a CFP100 Particulate Filter, or a CTCF50 Riot Agent Filter.

Aspect 8-6. The hybrid filter of Aspect 8-1, wherein the neck of the first housing portion includes internal threads configured to rotatably mate with external threads on the neck of the filter insert.

Aspect 8-7. The hybrid filter of Aspect 8-1, wherein the neck of the first housing portion and the neck of the filter insert are configured for a male/female or nested arrangement when the neck of the first housing portion receives the neck of the filter insert.

Aspect 8-8. The hybrid filter of Aspect 8-1, wherein the airflow exit of the first housing portion is coaxial with the airflow inlet of the second housing portion when the second housing portion is removably coupled to the first housing portion.

Aspect 8-9. The hybrid filter of Aspect 8-1, wherein the first housing portion and the second housing portion contain corresponding threads that rotatably mate together when the second housing portion is removably coupled to the first housing portion.

Aspect 8-10. The hybrid filter of Aspect 8-1, wherein the second housing portion includes the at least one of the electro-ionic filter or ozone generator disposed therein.

Aspect 8-11. The hybrid filter of Aspect 8-1, further including a gasket disposed between the filter insert and the first housing portion when the filter insert is removably coupled to the first housing portion.

Aspect 9-1. A housing cap configured receive a filter insert therein and configured to couple to a gas mask, the housing cap including: a neck having internal threads and external threads, the internal threads configured to rotatably mate with corresponding external threads on a neck of the filter insert such that the filter insert is removably coupled to the neck of the housing cap; and a body extending from the neck, the body having an attachment mechanism configured to removably couple the body of the housing cap to a housing.

Aspect 9-2. The housing cap of Aspect 9-1, wherein the attachment mechanism is a threaded connection.

Aspect 9-3. The housing cap of Aspect 9-1, further including a gasket disposed on an internal surface of housing cap such that the gasket is positioned between the filter insert and the housing cap when the filter insert is removably coupled to the housing cap.

Aspect 9-4. The housing cap of Aspect 9-1, wherein at least a portion of sidewalls of the body abut at least a portion of the filter insert when the filter insert is removably coupled to the housing cap.

Aspect 9-5. The housing cap of Aspect 9-1, wherein at least a portion of a base of the body abuts at least a portion of the filter insert when the filter insert is removably coupled to the housing cap.

Aspect 10-1. A method of factory assembling a hybrid filter, the method including: removably coupling a filter insert to a first housing portion such that an outlet of the filter insert is coaxial with an airflow exit of the first housing portion; and removably coupling a second housing portion to the first housing portion, wherein the second housing portion includes at least one of an electro-ionic filter or an ozone generator disposed therein.

Aspect 10-2. The method of Aspect 10-1, wherein the at least one of the electro-ionic filter or the ozone generator are arranged in series with the filter insert when the filter insert and the second housing portion are both removably coupled to the first housing portion.

Aspect 10-3. The method of Aspect 10-1, wherein removably coupling the filter insert to the first housing portion includes rotatably mating external threads on a neck of the filter insert with corresponding internal threads in a neck the first housing portion.

Aspect 10-4. The method of Aspect 10-1, wherein removably coupling the second housing portion to the first housing portion includes rotatably mating corresponding threads on each of the second housing portion and the first housing portion.

Aspect 10-5. The method of Aspect 10-1, further including positioning a gasket between the filter insert and the first housing portion.

Aspect 10-6. The hybrid filter of Aspect 10-1, wherein the filter insert is manufactured by Avon protection or an equivalent.

Aspect 10-7. The method of Aspect 10-1, wherein the filter insert includes at least one of a CBRNCF50 Filter, a FM61EU CBRN Filter, a GPCF50 CBRN Filter, a CFP100 Particulate Filter, or a CTCF50 Riot Agent Filter.

Aspect 10-8. The method of Aspect 10-1, wherein the airflow exit of the first housing portion is coaxial with an airflow inlet of the second housing portion when the second housing portion is removably coupled to the first housing portion.

Aspect 10-9. The method of Aspect 10-1, wherein removably coupling the filter insert to the first housing portion includes abutting at least a portion of the filter insert to sidewalls of the first housing portion.

Aspect 10-10. The method of Aspect 10-1, wherein removably coupling the filter insert to the first housing portion includes abutting at least a portion of the filter insert to a base of the first housing portion.

Aspect 11-1. A method of field assembling a hybrid filter, the method including: removably coupling a first housing portion of the hybrid filter to a gas mask, wherein the hybrid filter includes a filter insert removably coupled to the first housing portion such that an outlet of the filter insert is coaxial with an airflow exit of the first housing portion, wherein the hybrid filter includes a second housing portion removably coupled to the first housing portion, wherein the second housing portion includes at least one of an electro-ionic filter or an ozone generator disposed therein.

Aspect 11-2. The method of Aspect 11-1, further including removably coupling the filter insert to the first housing portion.

Aspect 11-3. The method of Aspect 11-2, wherein removably coupling the filter insert to the first housing portion includes rotatably mating external threads on a neck of the filter insert with corresponding internal threads in a neck the first housing portion.

Aspect 11-4. The method of Aspect 11-2, wherein removably coupling the filter insert to the first housing portion includes abutting at least a portion of the filter insert to sidewalls of the first housing portion.

Aspect 11-5. The method of Aspect 11-2, wherein removably coupling the filter insert to the first housing portion includes abutting at least a portion of the filter insert to a base of the first housing portion.

Aspect 11-6. The method of Aspect 11-1, further including removably coupling the second housing portion to the first housing portion.

Aspect 11-7. The method of Aspect 11-6, wherein removably coupling the second housing portion to the first housing portion includes rotatably mating corresponding threads on each of the second housing portion and the first housing portion.

Aspect 11-8. The method of Aspect 11-1, wherein the airflow exit of the first housing portion is coaxial with an airflow inlet of the second housing portion when the second housing portion is removably coupled to the first housing portion.

Aspect 11-9. The method of Aspect 11-1, further including removing the second housing portion from the first housing portion.

Aspect 11-10. The method of Aspect 11-9, further including removably coupling a new second housing portion to the first housing portion, the new second housing portion including at least one of an electro-ionic filter or an ozone generator disposed therein.

Aspect 11-11. The method of Aspect 11-1, wherein the electro-ionic filter or the ozone generator are arranged in series with the filter insert when the filter insert and the second housing portion are both removably coupled to the first housing portion.

Aspect 11-12. The method of Aspect 11-1, further including positioning a gasket between the filter insert and the first housing portion.

Aspect 11-13. The hybrid filter of Aspect 11-1, wherein the filter insert is manufactured by Avon protection or an equivalent.

Aspect 11-14. The method of Aspect 11-1, wherein the filter insert includes at least one of a CBRNCF50 Filter, a FM61EU CBRN Filter, a GPCF50 CBRN Filter, a CFP100 Particulate Filter, or a CTCF50 Riot Agent Filter.

Aspect 12-1. An electronic component attachable to a housing of a filter element, the filter element including at least one of an activated carbon-based filter or a particulate filter surrounded by the housing, the housing including an airflow exit and an airflow intake opposite the airflow exit, the airflow exit adapted to removably couple to a gas mask, the electronic component including: at least one of an electro-ionic filter or an ozone generator supported within a casing, the casing adapted to removably attach to housing such that the electronic component is adjacent the airflow intake and the at least one of the electro-ionic filter or ozone generator are in series with the at least one of the activated carbon-based filter or the particulate filter.

Aspect 12-2. The electronic component of Aspect 12-1, further including mechanical attachment features that facilitate the removable attachment of the casing to the housing, the mechanical attachment features including at least one of threads, bolts, latches, screws, interference fitments, pins, keys, bayonet coupling arrangements, twist-on-off engagements.

1.-59. (canceled)

60. A hybrid filter attachable to a mask and for use with a filter insert including an inlet to receive airflow and an outlet to discharge the airflow, the filter insert including an activated carbon-based filter disposed therein, the hybrid filter comprising:
a housing;
a housing cap removably couplable to the housing;
a chamber configured to receive the filter insert, wherein the filter insert would be removably couplable to at least one of the housing or the housing cap; and
at least one of an electro-ionic filter or ozone generator in series with the chamber.

61. The hybrid filter of claim 60, wherein the housing includes an air inlet and the housing cap includes an air outlet.

62. The hybrid filter of claim 60, wherein the housing and the housing cap each contain corresponding threads, wherein the housing cap is removably coupled to the housing when the corresponding threads are rotatably mated together.

63. The hybrid filter of claim 60 wherein the filter insert and the housing cap each contain corresponding threads, wherein the filter insert is removably coupled to the housing cap when the corresponding threads are rotatably mated together.

64. The hybrid filter of claim 60, wherein the electro-ionic filter includes an emitter at least partially surrounded by a collector.

65. The hybrid filter of claim 60, wherein the filter insert is manufactured by Avon protection or an equivalent.

66. The hybrid filter of claim 60, wherein the filter insert comprises at least one of a CBRNCF50 Filter, a FM61EU CBRN Filter, a GPCF50 CBRN Filter, a CFP100 Particulate Filter, or a CTCF50 Riot Agent Filter.

67. The hybrid filter of claim 60, further comprising a gasket disposed between at least one of the filter insert and the housing or the filter insert and the housing cap when the filter insert is removably coupled thereto.

68. A hybrid filter having a housing configured to removably receive a filter insert therein such that the filter insert is removably coupled to the housing, the filter insert having an inlet and a neck defining an outlet, the hybrid filter comprising:
a first housing portion including a neck defining an airflow exit, the neck of the first housing portion configured to receive the neck of the filter insert therein such that the airflow exit of the first housing portion is coaxial with the outlet of the filter insert;
a second housing portion defining an airflow inlet, the second housing portion configured to removably couple to the first housing portion; and
at least one of an electro-ionic filter or ozone generator in series with the filter insert when the filter insert is removably coupled with the housing.

69. The hybrid filter of claim 68, further comprising the filter insert.

70. The hybrid filter of claim 69, wherein the filter insert includes an activated carbon-based filter disposed therein.

71. The hybrid filter of claim 68, wherein the filter insert is manufactured by Avon protection or an equivalent.

72. The hybrid filter of claim 68, wherein the filter insert comprises at least one of a CBRNCF50 Filter, a FM61EU CBRN Filter, a GPCF50 CBRN Filter, a CFP100 Particulate Filter, or a CTCF50 Riot Agent Filter.

73. The hybrid filter of claim 68, wherein the neck of the first housing portion includes internal threads configured to rotatably mate with external threads on the neck of the filter insert.

74. The hybrid filter of claim 68, wherein the neck of the first housing portion and the neck of the filter insert are configured for a male/female or nested arrangement when the neck of the first housing portion receives the neck of the filter insert.

75. The hybrid filter of claim 68, wherein the airflow exit of the first housing portion is coaxial with the airflow inlet of the second housing portion when the second housing portion is removably coupled to the first housing portion.

76. The hybrid filter of claim 68, wherein the first housing portion and the second housing portion contain corresponding threads that rotatably mate together when the second housing portion is removably coupled to the first housing portion.

77. The hybrid filter of claim 68, wherein the second housing portion includes the at least one of the electro-ionic filter or ozone generator disposed therein.

78. The hybrid filter of claim 68, further comprising a gasket disposed between the filter insert and the first housing portion when the filter insert is removably coupled to the first housing portion.

79. A housing cap configured receive a filter insert therein and configured to couple to a gas mask, the housing cap comprising:
a neck having internal threads and external threads, the internal threads configured to rotatably mate with corresponding external threads on a neck of the filter insert such that the filter insert is removably coupled to the neck of the housing cap; and
a body extending from the neck, the body having an attachment mechanism configured to removably couple the body of the housing cap to a housing.

80. The housing cap of claim 79, wherein the attachment mechanism is a threaded connection.

81. The housing cap of claim 79, further comprising a gasket disposed on an internal surface of housing cap such that the gasket is positioned between the filter insert and the housing cap when the filter insert is removably coupled to the housing cap.

82. The housing cap of claim 79, wherein at least a portion of sidewalls of the body abut at least a portion of the filter insert when the filter insert is removably coupled to the housing cap.

83. The housing cap of claim 79, wherein at least a portion of a base of the body abuts at least a portion of the filter insert when the filter insert is removably coupled to the housing cap.

84.-109. (canceled)
